SEQUENCE LISTING (10 sequences)

SEQ ID NO: 1 - protein, 533 aa, Homo sapiens
MAFANLRKVLISDSLDPCCRKILQDGGLQVVEKQNLSKEELIAELQDCEGLIVRSATKVTADVINAAEKLQVVGRAGTGVDNVDLEAATRKGILVMNTPNGNSLSAAELTCGMIMCLARQIPQATASMKDGKWERKKFMGTELNGKTLGILGLGRIGREVATRMQSFGMKTIGYDPIISPEVSASFGVQQLPLEEIWPLCDFITVHTPLLPSTTGLLNDNTFAQCKKGVRVVNCARGGIVDEGALLRALQSGQCAGAALDVFTEEPPRDRALVDHENVISCPHLGASTKEAQSRCGEEIAVQFVDMVKGKSLTGVVNAQALTSAFSPHTKPWIGLAEALGTLMRAWAGSPKGTIQVITQGTSLKNAGNCLSPAVIVGLLKEASKQADVNLVNAKLLVKEAGLNVTTSHSPAAPGEQGFGECLLAVALAGAPYQAVGLVQGTTPVLQGLNGAVFRPEVPLRRDLPLLLFRTQTSDPAMLPTMIGLLAEAGVRLLSYQTSLVSDGETWHVMGISSLLPSLEAWKQHVTEAFQFHF

SEQ ID NO: 2 - DNA, 2021 bp, Homo sapiens
  mRNA (1)..(2021): mRNA sequence of PHGDH
gcagggatttggcaacctcagagccgcgaggaggaggcggagtcgcggagagtttgagtatttccgtccaatcaaaaggagactgtaagaggaggaggaggaggagatgactggggagcgggagctggagaatactgcccagttactctagcgcgccaggccgaaccgcagcttcttggcttaggtacttctactcacagcggccgattccgaggccaactccagcaatggcttttgcaaatctgcggaaagtgctcatcagtgacagcctggacccttgctgccggaagatcttgcaagatggagggctgcaggtggtggaaaagcagaaccttagcaaagaggagctgatagcggagctgcaggactgtgaaggccttattgttcgctctgccaccaaggtgaccgctgatgtcatcaacgcagctgagaaactccaggtggtgggcagggctggcacaggtgtggacaatgtggatctggaggccgcaacaaggaagggcatcttggttatgaacacccccaatgggaacagcctcagtgccgcagaactcacttgtggaatgatcatgtgcctggccaggcagattccccaggcgacggcttcgatgaaggacggcaaatgggagcggaagaagttcatgggaacagagctgaatggaaagaccctgggaattcttggcctgggcaggattgggagagaggtagctacccggatgcagtcctttgggatgaagactatagggtatgaccccatcatttccccagaggtctcggcctcctttggtgttcagcagctgcccctggaggagatctggcctctctgtgatttcatcactgtgcacactcctctcctgccctccacgacaggcttgctgaatgacaacacctttgcccagtgcaagaagggggtgcgtgtggtgaactgtgcccgtggagggatcgtggacgaaggcgccctgctccgggccctgcagtctggccagtgtgccggggctgcactggacgtgtttacggaagagccgccacgggaccgggccttggtggaccatgagaatgtcatcagctgtccccacctgggtgccagcaccaaggaggctcagagccgctgtggggaggaaattgctgttcagttcgtggacatggtgaaggggaaatctctcacgggggttgtgaatgcccaggcccttaccagtgccttctctccacacaccaagccttggattggtctggcagaagctctggggacactgatgcgagcctgggctgggtcccccaaagggaccatccaggtgataacacagggaacatccctgaagaatgctgggaactgcctaagccccgcagtcattgtcggcctcctgaaagaggcttccaagcaggcggatgtgaacttggtgaacgctaagctgctggtgaaagaggctggcctcaatgtcaccacctcccacagccctgctgcaccaggggagcaaggcttcggggaatgcctcctggccgtggccctggcaggcgccccttaccaggctgtgggcttggtccaaggcactacgcctgtactgcaggggctcaatggagctgtcttcaggccagaagtgcctctccgcagggacctgcccctgctcctattccggactcagacctctgaccctgcaatgctgcctaccatgattggcctcctggcagaggcaggcgtgcggctgctgtcctaccagacttcactggtgtcagatggggagacctggcacgtcatgggcatctcctccttgctgcccagcctggaagcgtggaagcagcatgtgactgaagccttccagttccacttctaaccttggagctcactggtccctgcctctggggcttttctgaagaaacccacccactgtgatcaatagggagagaaaatccacattcttgggctgaacgcgggcctctgacactgcttacactgcactctgaccctgtagtacagcaataaccgtctaataaagagcctacccccaactccttctgcaaaaaaa

SEQ ID NO: 3 - protein, 370 aa, Homo sapiens
MDAPRQVVNFGPGPAKLPHSVLLEIQKELLDYKGVGISVLEMSHRSSDFAKIINNTENLVRELLAVPDNYKVIFLQGGGCGQFSAVPLNLIGLKAGRCADYVVTGAWSAKAAEEAKKFGTINIVHPKLGSYTKIPDPSTWNLNPDASYVYYCANETVHGVEFDFIPDVKGAVLVCDMSSNFLSKPVDVSKFGVIFAGAQKNVGSAGVTVVIVRDDLLGFALRECPSVLEYKVQAGNSSLYNTPPCFSIYVMGLVLEWIKNNGGAAAMEKLSSIKSQTIYEIIDNSQGFYVCPVEPQNRSKMNIPFRIGNAKGDDALEKRFLDKALELNMLSLKGHRSVGGIRASLYNAVTIEDVQKLAAFMKKFLEMHQL

SEQ ID NO: 4 - DNA, 2221 bp, Homo sapiens
  mRNA (1)..(2221): mRNA sequence of PSAT
ggccaggaacgccagccgttcacgcgttcggtcctccttggctgactcaccgccctggccgccgcaccatggacgcccccaggcaggtggtcaactttgggcctggtcccgccaagctgccgcactcagtgttgttagagatacaaaaggaattattagactacaaaggagttggcattagtgttcttgaaatgagtcacaggtcatcagattttgccaagattattaacaatacagagaatcttgtgcgggaattgctagctgttccagacaactataaggtgatttttctgcaaggaggtgggtgcggccagttcagtgctgtccccttaaacctcattggcttgaaagcaggaaggtgtgctgactatgtggtgacaggagcttggtcagctaaggccgcagaagaagccaagaagtttgggactataaatatcgttcaccctaaacttgggagttatacaaaaattccagatccaagcacctggaacctcaacccagatgcctcctacgtgtattattgcgcaaatgagacggtgcatggtgtggagtttgactttatacccgatgtcaagggagcagtactggtttgtgacatgtcctcaaacttcctgtccaagccagtggatgtttccaagtttggtgtgatttttgctggtgcccagaagaatgttggctctgctggggtcaccgtggtgattgtccgtgatgacctgctggggtttgccctccgagagtgcccctcggtcctggaatacaaggtgcaggctggaaacagctccttgtacaacacgcctccatgtttcagcatctacgtcatgggcttggttctggagtggattaaaaacaatggaggtgccgcggccatggagaagcttagctccatcaaatctcaaacaatttatgagattattgataattctcaaggattctacgtttgtccagtggagccccaaaatagaagcaagatgaatattccattccgcattggcaatgccaaaggagatgatgctttagaaaaaagatttcttgataaagctcttgaactcaatatgttgtccttgaaagggcataggtctgtgggaggcatccgggcctctctgtataatgctgtcacaattgaagacgttcagaagctggccgccttcatgaaaaaatttttggagatgcatcagctatgaacacatcctaaccaggatatactctgttcttgaacaacatacaaagtttaaagtaacttggggatggctacaaaaagttaacacagtatttttctcaaatgaacatgtttattgcagattcttcttttttgaaagaacaacagcaaaacatccacaactctgtaaagctggtgggacctaatgtcaccttaattctgacttgaactggaagcattttaagaaatcttgttgcttttctaacaaattcccgcgtattttgcctttgctgctactttttctagttagatttcaaacttgcctgtggacttaataatgcaagttgcgattaattatttctggagtcatgggaacacacagcacagagggtaggggggccctctaggtgctgaatctacacatctgtggggtctcctgggttcagcggctgttgattcaaggtcaacattgaccattggaggagtggtttaagagtgccaggcgaagggcaaactgtagatcgatctttatgctgttattacaggagaagtgacatactttatatatgtttatattagcaaggtctgtttttaataccatatactttatatttctatacatttatatttctaataatacagttatcactgatatatgtagacacttttagaatttattaaatccttgaccttgtgcattatagcattccattagcaagagttgtaccccctccccagtcttcgccttcctctttttaagctgttttatgaaaaagacctagaagttcttgattcatttttaccattctttccataggtagaagagaaagttgattggttggttgtttttcaattatgccattaaactaaacatttctgttaaattaccctatcctttgttctctactgttttctttgtaatgtatgactacgagagtgatactttgctgaaaagtctttcccctattgtttatctattgtcagtattttatgttgaatatgtaaagaacattaaagtcctaaaacatctaaaaaaaaaaaaaaaaaaaaaaaaaaaaaaaaaaa

SEQ ID NO: 5 - protein, 225 aa, Homo sapiens
MVSHSELRKLFYSADAVCFDVDSTVIREEGIDELAKICGVEDAVSEMTRRAMGGAVPFKAALTERLALIQPSREQVQRLIAEQPPHLTPGIRELVSRLQERNVQVFLISGGFRSIVEHVASKLNIPATNVFANRLKFYFNGEYAGFDETQPTAESGGKGKVIKLLKEKFHFKKIIMIGDGATDMEACPPADAFIGFGGNVIRQQVKDNAKWYITDFVELLGELEE

SEQ ID NO: 6 - DNA, 2142 bp, Homo sapiens
  mRNA (1)..(2142): mRNA sequence of PSPH
ggcgttggagctctttggggcccagctttgcggacccgggagctcgggacgcaggcggggcttgtgctccgcgggggcagggcgtagggtgggcctcctacctcccctgatctcgcggtttgttccgtttcattggagcttcccggaccgtgtgctcgacggtgccctaggtgccgtggggccacacgcgagtctgataagcaccctcccccggaatcatgcggtgctgtgaggcctagcgaagatgaagatagaatgcaaggtagaaagtgctggatacctttagaaagctgcaggactggtgcgatgggagttgagacgtaagaacctgcccgtccgtagggctctggatgctgctgaggcccgaggcccctatggcagatttgaaaattcacccttgtagagtcattcctgcctttgagcggactcccttttaagcagatctcaagagagcgttcggtggaggccctgggtctgcacagctcacctccctgggaactgctcgcccgagcgtcggagccggcgctggccccctgcagccggaaggttgcagccgcaggagccccggaggcccaggacacagggctcttgctcttgcagaatccacaggtctttcttgaggaaatctgtagacagaactttgtgctgcgtttttatctagggaaggaacagaagagtgtcgtctcctagaaatctagcactggagaaacgaggaaaattcttccagcgatggtctcccactcagagctgaggaagcttttctactcagcagatgctgtgtgttttgatgttgacagcacggtcatcagagaagaaggaatcgatgagctagccaaaatctgtggcgttgaggacgcggtgtcagaaatgacacggcgagccatgggcggggcagtgcctttcaaagctgctctcacagagcgcttagccctcatccagccctccagggagcaggtgcagagactcatagcagagcaacccccacacctgacccccggcataagggagctggtaagtcgcctacaggagcgaaatgttcaggttttcctaatatctggtggctttaggagtattgtagagcatgttgcttcaaagctcaatatcccagcaaccaatgtatttgccaataggctgaaattctactttaacggtgaatatgcaggttttgatgagacgcagccaacagctgaatctggtggaaaaggaaaagtgattaaacttttaaaggaaaaatttcattttaagaaaataatcatgattggagatggtgccacagatatggaagcctgtcctcctgctgatgctttcattggatttggaggaaatgtgatcaggcaacaagtcaaggataacgccaaatggtatatcactgattttgtagagctgctgggagaactggaagaataacatccattgtcgtacagctccaaacaacttcagatgaatttttacaagttatacagattgatactgtttgcttacagttgcctattacaacttgctatagaaagttggtacaaatgatctgtactttaaactacagttaggaatcctagaagattgctttttttttttttttaactgtagttccagtattatatgatgactattgatttcctggagaggttttttttttttttgagacagaatcttgctctgttgcccaggctggagtgcagtggcgcggtctcggctcactgcaagctctgcctcccaggttcacgccattctcctgcctcagcctcccgagtagctgggactacaggcacccgccaccacatccggctaattttttgtatttttagtagagacggggtttgaccgtgttagccaggatggtcttgatctcctgaccttgtgatccgcctgcctcagcctcccaaagtgctgggattacaggcttgggccaccgcgcccagccaatgtcctagagagttttgtgatctgaattctttatgtatatttgtagctatatttcatacaaagtgctttaagtgtggagagtcaattaaacacctttactcttagaaatacggattcggcagccttcagtgaatattggtttctctttggtatgtcaataaaagtttatccgtatgtcagaacggatttgtggaaaaaaaaaaaaaaaaaaaaaaaaaa

SEQ ID NO: 7 - DNA, 39431 bp, Homo sapiens
aatactatgcagccataaaaaatgatgagttcatgtcctttgtagggacatggatgaaattggaaatcatcattctcagtaaactatcgcaaggacaaaaaaaccaaacaccgcatgttcccactcataggtgggaattgaacaatgagaacacatggacacaggaaggggaacatcgcacaccgggaccttttgtggggtggggggaggtggggagggatagcattaggagatatacctaatgttaaatgacgagttaatgggtgcagcacaccagcatggcacatgtatacatatgtaacaaacctgcacgttgtgcacatgtaccataaaacttaaaatataataaaaaaaataataaataaataaataagtactgaaggatggggctgccttgccctccccagcaaaaaaaaaaaaaaaaaaaaaaaagttaattcttccgtgatttttgtgccgtttatgtttttagcttttacatttcacatgtaaattatttgaaatttgtcttggcataaaattcagatttgactttattttctagatgactgtccacttgttcaaacaatatttattgaatgattctctatggagtgtatttatggtaatagccaaatgtaaggaattgtcgacggggagaaaagtgaagtttaatcaagattgtcagtgaagatatgcttggctaagccatttctttttgtcctcttctccctcctttcctttcttctgctttcttcccctctcattctcttctgttctttgtttttctctttttgtcgtattcatatgtaacttgattcattctctttggggatagaaacatttttaaattttcttacgaattaaaaaattctttaaatttgtaaaaaaaaaaaaagttccctctgagtagggactacatgtgacttgttcagcattgttattccagtgcctggcacatagtcagtactatagaaatatttgggcaggtttcttgaatgtgccacacttgttctgtttttccttctgcctcgaattctctttttctttaattctttgcatagccagtttcttcttatttcagtctcaacccaagctccaccttttcaggaaggcctttcctcatcataaagtctatgattcctctcccctaacctgtctttcacatcatctggttttacattatttattatatatgccctgagattatccagttcaattttttgtttatttatttattttctatcctcaaacgcaatgtaagctccatgagagcagagacggtcttaatcaaagcttcatttccaaccccctcagcagtgcctgacacattacagtttcaataaatatacactgattgagtggatgaatgaaggaaagtcaagcactagagattacactcacttgtatgctttgccttatttaatccctacaaaagccttatgaagtaagcactatcatactattcattccatattacataggagaaaactaagtcttaggggggcaatgtaatttgcccaaaatcacattgttcataacttgagaagctgaaattcaaatcaagatatgcctgttttacggatgacagttcattttctgcatcttaaaatatcattttatttctttttttattatactttaagttctagggtacatgtgcacgacatgcaggtttgttacatattttatttcttatgattcgagtttggatatcacagctggtctccaagcagaaaaagagagttcctactctcctccccttaaccccattaactttatcctcagagttacaggcggaacagctggacatcttgcaaccttaggtaggggtaggtcttgccttgacagattcactacacacaaggaagcaggagtgtgtacattattttaggcttccagcttcgaaagagagagccaaaaggggctggcctgggcacgcactaggaccttttccccaaacttcactaatcagagtggttttctcttctgaaggggttggggccagaggaagggagagaaggaggggtctgaggagaagggcagaactgttttcctactacttccaagctggccaaagttcctgttttatggatggatgcaataacacagggagagggagaaatttccccaactccaaccctcagttatcaaagaaaaacccttagtttgcaattgatgttggagattctggggtgtagtggtgagggatatatctgtattggcagtatcaggtctcagcccgaccagcaacacatttgatgatatcgcttctaaaaatcactcggtacctctgtactaactttggagtcaagtttaaactcctcctcagggcaagacacttcatgagcagcccctggcccctctgcagtctcacctcctggtatcccactggcatcccccacagccatggtagagaagccatgaacatgttgcaactcctcacaactctaagcctttggaaatgtcatttcttctcccccggctaattcatattcagctctcaagattaggcagttaaccatgagcagcttcaaggcaggttatgttttacctatctctgtatccttgagcctctcaaagtagctgccttgaatgagtttcaaaaggttagctgaaaggcaaccttgcttggaaatcctcctggcacctcaggagatggttctcctccttgctcccaaagccctccagcacacctttatttataattcttaaactatacctttttaattttttttattattttttagagacaggtctcactctgttgcccaggttggagtgcagtggcacaatcatggttcactgcagcctcgaactcctgggctcaagcaatcctcctgcctcagcctcccaagtagataggactacagcaatgtgccaccatgccggggtactcttcttaaaatgttgttcttttttctttttgaggaaagaaggctgtgtcttatgtgtcacagggcctagcccataggaggtactggataaattaatttttgaatcaaacctcttctgagcctctgtgatgtggaaggcatggtgctaggcactactgggggtatacaaaatcgtgtctctgatttaaggaaatgagagttcatggggaacaagacatgaaaagttcataaatatattgcttaaggcactgtgagataacttgaagttaattctatagaaatttaaaaggagggaacatttctgacttatgtaacaattatcacacaccctccttttcacttggaaatggactcttaatctgttaattattttcttaacggtttagaagtgtcttttattgtgtggcttggatctgtttaaaaagtaggtgggtcataaataataaataaagaaaacaggtgatcaggcttaggaggccctctgggttgtgcgttcagacagtatccaagaagtttgacttggtgtgaaagggtgggattacatgttaatgatagcagggtttagattctacactgaaaggaagcagttagtgagccctgggaggagttgaaggaaagcagttacaaaaatggatgcttcccaagaccctccattaagcctcctgggataggtcccctgtctgtccctagactccagggtactgctgggcacttcaagagagacagagaatttgaatactcatgagtcttggaatgtaggaataacaattcatccgaacaagcaaacatgatagacaatttttttttttacccactgaggacattgtgtctaaaaatctgatttgtgcccaggaattatctgtatagaattttcattactttatagaattgatattgcatagttctgtaggataaggatctagcacttgagttatgaatagctcaggatgacataacacacaatggttagttggactggatatgtccataaagctgaattctaattcaactatgagcatctctgattctgcttctgattctaggtgactttaatcccaaattgaattttctgacatttctgtaaaatggggcatgagagagcacgtcggttatggggtttaaatataaaagagttaagagctgagaagtgcctccttggtcaattaaaccacactgacatcccgttttccttttcttctttttcataattagagaggaagtggagggaaaggaaataaataactttaacaaagtatgcctagtttaaatgcaccaggtccccgctcttttataaactcataattctcattgcatcactaatttgaaattaatcacagctccttggtgtctcagaaagcttcctattgcttttcactacgtaactttccaagtctgtgtgtttggtactctaggatgttaggccaccagaggaaagaggatgtatcttttttttttttatactttaagttttagggtacatgtgcacaatgcgcaggttagttacatatgtatacatgtgccatgctggtgtgctgcacccattaactcgtcatttagcattaggtatatctcctaatgctatccctccccccctccccccaccccacaacagtccccagagtgtgatgttccccttcctgtgtccatgtgttctcattgttcaattcccacctatgagtgagaatatgcggtgtttggttttttgttcttgcgatagtttactgagaatgatgatttccaatttcatccatgtccctacaaaggacatgaactcatcattttttatggctgcatagtattccatggtgtatatgtgccatattttcttaatccagtctatcattgttggacattttctttttgatagtcgaattcccttcaacgtctagcacagtgccttgcacatttacccactcaaaaaaaatttcgtaaggaattaataaatgaatccttaggaggaaaagtgaaaatgaagtttttctctcaggatgaggtgtatttctccgttcatttcagatatgcatcagctagtcagcgtaaattgtgctttttatatgctcaccaggtgtaggtaagagctttggctgagatggagaaattcatcgcgggaggataataaagcgggcagggatttggcaacctcagagccgcgaggaggaggcggagtcgcggagagtttgagtatttccgtccaatcaaaaggagactgtaagaggaggaggaggaggagatgactggggagcgggagctggagaatactgcccagttactctagcgcgccaggccgaaccgcagcttcttggcttaggtacttctactcacagcggccgattccgaggccaactccagcaatggcttttgcaaatctgcggaaagtgctcatcagtgacagcctggacccttgctgccggaagatcttgcaagatggagggctgcaggtggtggaaaagcagaaccttagcaaagaggagctgatagcggagctgcaggtaaggcgagagagagaaaattgaggtctctagggcaacctccatggaaaaaggctggctgcgcccaggccagcgcgcccccctcgcatgcaccccgtatcaattagttccggggcctcctgagattggggggtagagaagaacgggggcgggaggaggcagaaagagggaagaacaaacggcggcgagatgcaaacttttcttttagtttgcaaccgcgtctttcacgttggcatgcctccgctagcattgcaaagtgcgggctgctccaactggtcctgcaggctgctcgcggatgccagcgcgggatgccagcgcggcgccccagcgccttagcgcgcaattgttctggcagcctcgcgccgcctctcccccaacccccaaccgcctccgcgcggggcgatcgggagaggggccccaaagtggcttctttgcggggaacccagggactggcgattcttcccaaccaagttctgggccgccccgccgattctagcctgccttggcggtggcggggttgggggggtcggggggcggggggaagctggcggagacccaaccagtctggtggctgctggggcagctggaggggaaggcagccctcgtaaggcagcaaacacgtacccgcccctcgtctgatgcaagactgctccgtgctttcgccgcccctctgcctcctgggagccttcggaggaaagggaggcgggcggggagcgctggggtccagatttagcctcctccccacctttggtttatcgctggttgggagtgaccagactcgactagaatccgatcccaaacgctcccagatgatttatagtcttagcaaatttttatctccttttgttgtgatatataattaatctacttcaaatctttatccaccgtgtttgaaaaggcctgctgggcctggtggccttgtccggaatatttattttgtgaactcttcactccagtgcctgcactttcgacctctgtagtcgacccagctgcccagtctctacctcacctgatccagtatattctcccccaccctccaaatcgcacaaccgccttctgcccgcctcagccctgtccaccaccttcagccgtctcctcctccgaggaccccttagaccgcagaggctgctgttgttgctttagttgtgccacaaccacgtggtcctggaaactgcctcgctcactttgtctgcctttgtatctgctggcgtggatcttgtcaccatggcatgacactatatccaagtatccagctctgccctgtactcagaagaatttggtccctgccttcaagaagtttgtaagctggttagggagcatggctaatacgcaggagacagttgagcagatgacacaagagcatagccccgaattagaccatccagctccctaacctttgcgtgttccccttctggaaaagccagagaaaggtccaatcatcatgcacagaattggtgggcaagggcttaggacttgagtaggattggacgggtggcaggctcctcacatctgtcacctcctttcttgtgtgaaattgtatcagtggctctggggaacaaccaggaaaggcagtggggtgtcctgcagaacagcctctgactctcacctccaccagagcaccaggttgggaagagggtggcaatcaagtgctttcacctctctaaaggaaattagaaacctgataccccagtgagggggtggggatagggagggatgagggcagaattgagaggaatgggaggcctcatccataatgaggggttgttccatgaagtcaggaatcagctgggtggatgctgggagtctggtgctgaaacttggagaacattttaaaggcgctgaaaactcttggcaggagaggaggagtgtttgttggctaacttgactgctgggcatttttggactgtttgggagggctcagctttcttgtctgtctttgcaacacatttggttcagaatgccaattaatctccttggtcagcccactgaagggtcctcatttctacactatgcctttttattctgcatgaagaggtgtctggcatagtgtctcctgccctcccccactgaagtctctttaatgctgaaggaagcttcggcagcgttgctagaaccgggcctggccttggttcccactgccttgtattctgggcaggcggtctcctctcactcaccccctggtcaggaagtacaatcattcctccctgccctttcagtggtgcttttcagacttggagcaagtcatagttcagtttaaggggtcatgacgagcattttaaaaaacaaagtaggaaatatcagacagcaatgcagatagatagtaaggttgtaagtattgtttcatgaattttgtttcatttatgttgtgtagttgtatgtgtgtggcatgtgagtatgaaattacaacttaaaaatgtgtttcttggccgggcgcggtggctcacgcctgtaatcccaacactttgggaggggccaaaggaggcagatcatttgaactgaggaggttgagaccagactgggcaacatggcaaaaccctgtctctacaaaaaatacgaacattagctaagcatcttgcacaagcctgtagtctcagctactgggggatggggtggagggctgaggccctggttgctgtcactcttcccaacctggtgctctagtgggaggatcgcttgagcctaggagtttgaggctgcagtaagccatgatctcgccactgcaatccagcctgggcaacagagctagaccctgtcccacccctccaccaacaaagatgtgtttcttacaatgaacctcagtcaaaaaagtttggaaaacgttggcttaaaataataaagcatcttgtttggaactcacatcttaaatatacctaatatgccggaaaatatttaaataatattaccatgtatcagtggcttttaaactttgactgtgacccacaataagaaatttgttttatgtcataacttaaacactcatacatatgtgtacacacagacacacacaaattttatgaaataatacttactctttttacatatgatgcattctggtattttcttctgtttcattaagtagcattgctggtcaggaccactaaactaaacactggtaatggtatttaatttactgaagcacatccataccttcatatcagtacttgaagaaagattaagaagggaataaatctttccatgccaaatttcttttcaaatttctttaaaataggaagttcccagcctttacacaaggatttagtttgcaaaagttaaataaccatcaaattaatcaggatcacacatcttgtgcaaaacagggtagacctgccttccctatagctggggaacatgacctggatagtttcattgtcttgccctatgatttaagtcagaggcatggcaatgctagaattacctcctaggccaagcttgtccccttgggaaaggaagcatgtgtgaacctgaggaatggcaatggattcccattgctgagttagcagtttgtctcgaacagccataaatcaagcagttttctagctgaacagcagctaaggacaggggagcgggaggacttgtaggaagtgtgggaagtgcctgttcccgccacagtgcagacacagaagccacacgcagagggcagtgaacatcagaatttggtgctgtgtagagagagacttgcttcctcacccaaggaatgacaaagagaggggaggatctctggacactggagaagttttaatcgcaaactgcagaggatgtgagcagatgggtgcatttggttagggctgtggtttccataagtagaggacaggagcagacagcttggggcaaagtctttgaagggggtgcgttgtggcgtgaggagggaaggggcttgggtgcaggggctttgagggtccagcaggaggtaggttggattcaggctgtctgtgaatatgtgctgattgaatgagtggatagagtgggtagttgaggttgtaagaggccttggaagctcattaactctgactgatgtgggatgtctggttctaatgtgggaccaaggtctcctgagtgacagtttctgcccttgggagaggtgagacctgatgctcatctgtaggagtctgactggggactcttagtttccctggcaccaaagaaggctggtttgagggaaaagtgaagagactacagcataaatctttacaagggagatccatccagagagtccatgctcatgaaagaaacaatagtgctgtttctgtgcttgttcaggggctacaagaatgggtggactttggggagtctgcatgctctccagctagtgttgcctgtggggagcaggtttgcaaggtttggggagcattttggcagctttggagagaacattgaagaaggatggacccttaaggcccaagggagtgattggtagagcctctggagcaggactgggcctgagatgtgggctctggaacttgatgctagagagacaaggtgataagatgatgactctgggtggtcccttctcctcttgctccatgcctgcttcttgtcagttgaactttgacttttggtcctgagctgaccacttgaaaggaggttgtgtttcctcagtgtgttaccaattttctaaggtgtcagtgctggctgcaaagaaatgaccactaaaaatgtgcttacccacccactgcccagttgccctccaaaaaggtcatgcagtgtacactcctgtttccctccatgctcaccaatgctgaatattattcattttctttctgccaaaatgatgcccaaaaatatcatatttaaaaatctatatttccttgatatctagcgttgaagagtctataaactagtgaaactagtgaccactttgtagttcttctgtgcattgcctatttatatatttatctattttttattgtgctctttatcctttaaaaaagtggatttgtggtgcatgcctataatcccagctactcgggaggctgaagcaggagaatcgcttgaacccgggaggcggaggttgcagtgagccaagatcgcgccattgcactccagcctgggcaataagagtgaaactctgtctcaaaaaaaaaaaaaaaaaaaaaaaaaaaaagttgatttgtaggaattctatcacttttggtgatgaatccttgttctgttacatatggtgaaaggagttttctccacatatcaggaccactacttgcttttgtttatgggatctttttccatgcagaagtttaaaatttgaatgtaactggatttgtcaatattttcttccagagtttctaagatttgtgccttgcttatgaaaggttttcccaatccgagtcataaaaatatcccctgacattttcttttaatgcttatatttttaaaaattctttaattcatctggatttttaaaaatctgaagtgaggtagcaatctattatttttcttcttgaattttcataacattatttcttgtttagttcttcctttccgcatcaatttgagttctgctttttattttctaaattctcacatattctgtttgtctagtgtctatttagttctgttgactgatttgtcaatttctgaacgtttttcctgctttttttcaacttcttttaataagatttttgaaaatccttgagcactcttagaagtagaccttatgcctccttatcacagtcaaaggaagggatttctgatgagcaattttcaggcatgtgaagtgttaaagttatttgggaaagagggtgaaggtgtgaggatgggggactgatgagggtggtttctaccggcctctcatcactgaattcatttattcaatggaagtgtataaaacacttcattgtgaagtatcaactgtgagcttgcctacaggccacaggcaacttgctcttctcgacagaggcatttgtgcctatttctattatcccccagtaggcaggctggctgggaggagcaggtcaccagaagatccagggacctttgttagggagcttctgccatcttggagaagccacaggatttctggcaatgtctctcctggtttaggtgtctaaatgctgtatttggaaggtagttggaactcaacagatttggacttgattccagcttctgccatatcctagctatgtgacttcagcaagttctctaaattctttaagtgtcagtttgcttgtctgtactatagggataattgtaatatctgtcttaggattgttgtgaagcacagtgctggatgtaaagtacttgctcaaaaaatggtagctattgttactagtaaatctctccaagggattttacagtttggtttttgcacctaactggccatggccttggctctggccctatcttcttgacatcttggcaggtctaccaactcttcttcagttacagtctcagagttagccagtggcaatgactttccagttcttggctggtttttgatctgtaagagtgaccttgggcaggttacctctcaaagcctctgtttctttatctggaaactggggatgattataccccttctccttggagttgtgaagagaataatgtaaagcaactggtgtaatgcctagtacccagcaggtgctcaataaatggtttatgtgctggtgaggctggtactatggatatatggtatagagtagataaagttttaatataaaatccctgcattaggcatagaaaatataactaaggagaccaaaactgcaggtctaagagaggacaactagatatgaaacatattatattggtgatcacaacaaaaatggtggggtgtagggagagtgacagttcagggttgttggcaaatgtttcctggaagataactttattgcaccttgaaaagtcagtaaagtgtgtccaggcagggaaatccttttctttggagcctgtgtggtggacttgaatgtggaaaagatgccagtatcctgtttctgcctggcttggctgatatttaactaggctgctatggggggaaatcagaatacctccaggtgctcacctggtgttggcctcagcactggggcagtcagcactgaggtggcttctgtttgattcctctaagccctgggtgcccatgtgacttgtatacacccaggtcataatcagtttagcataatgccagatacgtacatagcgggcatccgataaaatgaaggaagaaaagtacaggggaggggaagggaaagtaggcaggcttaactttgttgggcttcagtttttcctctgtaaaatggagttggaccagatgctggtgaacatttcttcaaatctcccactctgacgttctgcttcagaacctcagacataaaaggatttggagtgtcttccaggccgcctgcatgtttctctctgctggtaggcacagcccccaagcagtgaggatgtgtgatgcataaacacctccagaggaatcgttctactattgctcttgaacaattcttccagtgtttagaagctcttaaggcttaaatattctatgctgcagcctaagcatcattcctcttctcttcttagtggagataaaattacccactgctctccttacatttactttgtccatatttgctcctatgctctaggctcgtgcacaacaaacacagtgtgggcccttaccctagaagccaacttctcatgacctttctctatctccagaatccatgcagtgggaatgaaggtaaaagaaggttttcatgggatccagctgagagctctacggggaaaatggatctgaggagccatgtgctccatctcttttattttacaggtagagactaggggtatagagtgaggtgaattaccgcagtgacccacacattgttggcagacctaggattagaactctgtcttcctggttcccagcttggtgcttttgaaagcatacttgctgctttcttaccggcctggtgtctgccactttgggacagagtgtggacttgctcacctgccccatttcttagggattctcattctgtgtttgagcaagaatattcttattctggaaagaaccacataccacaggattctgggtgagcataaggaagattgtcttggggatctgacttagctcacgtatagtggctatgatgaattcagtgtcttattttttgcatatgtatatttttagtctaatattgcctgggtgtctgagcaagtctagatgaatttaattgctctcatttttcccctgcccctcttcctttggtctctcttttaggaaatgtttttctttcaacattcgtttcattcattatttactcattcggccaaccaacatttattgagtgccttccctgtatcagggacaggggcttacaaagtagaatttgatcccacctctgccctcagtagctcagtgtctaatggaggtagtgatgttcattaagcgtcgccagatactgtgctaggtgctgtgcctgttctctctcgcttgttcctcacacacttgagaaggccgaagctgattcatagcttggaaggcaggggccttggatttgaacccaggcctgaccaatggcagaacctatcagatgtgtggacagatgacattgcctttctttctttggatatatcaaaatcagccagcaggcaggaactcccattttgagcaagcaatgtgcaggaatgatagggtatacagagaggaacaggagatggcccctgacttccagcatgtgtctgatggacatccaggctgcaggcatcatggtgctgtctagagagatgagccaggtgcccagagcccatgggccaatgctgccctttcttgagcatgccaaacaaagcggttggtgtgttagaggcacagtctcctccactctaagtaaaaatcagcatgagtcctagcccacatttccctagtgagtataccaaagatatctatgaactggcagtcatcagtgacttcctaaggttccggaaatgcatctcttactcaggagtaagcaatgatgtgcctgcggctttacgagttctcacagaatgactttctggacccaaatgttttttctgcttcaggactgtgaaggccttattgttcgctctgccaccaaggtgaccgctgatgtcatcaacgcagctgagaaactccaggtggtgggcagggctggcacaggtgtggacaatgtggatctggaggccgcaacaaggaagggcatcttggttatgaagtaagtcatggaggctgcgggcggtttgggggtaggggggtgagtgcggagactgaccacacctagggagaaaaaactcacttgagagaaagctgagtccattggaagggcttccaggaggatgcctggtctagggcctgcatggtcaacacacacagcatagtggtttcaaggtttttggaaggcagctatgctcaccactatatcaccaatgccatcagggtgttacacagtttttgaaattgagagtccctgcataatctcaaaatgtttcacgagcccaccccatgcgcagttgcttgcacctctgtgacctggttcaggaatcggaaggtcagtgagttcatctgcatttcctgctcccacccagccccctctgcctctattaatgctgtttgtggcaggtttttgtcagctctactttactgtgcttgtacagaggcacaacctttgctagcagactaatgactagaatccttgccctccccacttccctgccaccttctggaactaagacactagtttctctttcagtgctctagggcaagaggagaagggtcccatttaaagctgtttctgcagaaacacaggggcagaggtcatcagcacgccagtgctgtactgtacccgttctgtcacttagatggtatggcaaggccatccccaggcctctttgttcttaagacttttctcttcccttggggacttcattgtccttaagacctttcccctcccctgcactgcacttccccctgtagggtagaggttaattggtcacctgactgaagtcaatattcaacagcagaaatgttaaacgataacccatcccacattcttgccttggacccagaggcagccaggccccaatctctgcacctctacttgcgcccccatacagcctgtttgctgtgggaggatgagaagccaggtggttttgcaggcagacagactctgagagtccgtttatcttatacaggatctcttgactttttcttcttgtaaccttattaaccttcattccagagatgaaaaagacagacccagtaggggaataatcagggtgaacacgtatatgaaattctttcaaaaacctaaaaagcatttaagaaaagaaaaatagttttgtgggttgccacctctatttttttgtttataaaatgggaagggtctggattgccctgtgacttagggtgtgaggaggctttctcagttcagaacttgttgagagataggaagagtagtcagacaggtggagattactaataaaagctaggttggggctgcgtgtgggggctcacacctgtaatcccagcactttggaaggccaaggtgggaggatcacttgggcccaggagtttgagaccagcctgggcaacaaagtgagacccccatctctacaaaaatacaaaaaaaaaaaaagaaggaaagaaaagccaggtatgttggcaccagcctgtagtctcagctactgagaaggttgagctggaaggattgcttgaaccctgggaagtcaaggctgcagtgagctgtgatcatgccagtacactccagcctgggtgacagagcgaggccttgtcaaaaaaaaaaaaaaaaagccaggttgggtaacttgatgaagatatgtaggcattggactgagccctgaattcgagagactctgaccttggtaagatcaatggtaggagcagcagggaatttgtgctttctggaggcaggtagatcctgaaataggagaaagaaaagggctgaaccacacacaaatctgatcatgtgagacacttctctgccttgggaggagtgtttggatagagagaagccagatatgtttctctaatggaggtccctctgcagggaaacataaagccaggggaagggggtttctttccagcccttggcttagggcgagagtacatcagagaactctggacagtggcgtggtcagttcatggagcaggagtgagagaacacagcctgcactcaaggctttctcttggggaaggagtgggaatactgggtctgtgcccattgatgtcccccttttctttgatctttagcacccccaatgggaacagcctcagtgccgcagaactcacttgtggaatgatcatgtgcctggccaggtaagtccctgacttctcagcaaagctagtctctccgatatgccaattattaccacttgccaagcaaatggacccagaaaaacttagaaacatttcgtctcagcgacttgcagactgctaagaaggcgacatgcagactgcaaagaacctttaaggccatcaggtcctctattcaacattcattacacaggttccctagtgcacaaaggtgcacttctctttactcatgttctattctccatgctgcagacaatctgccttctctagcctccaactcttgcaaatgttctccataccctgctgcctggagataacaaggaaggatgttccaggcagcagggtatggacagtgtctaggtaccctctgcttacttttcaagattaagcttcagtattaatttatccaagaagctctctggcttttccagctaggctatgtccctcactgattcccctggccctcagggcttatctctgtcactgcccttacaaatctgtattgtaatagaatggagtaagaacctgcttcagggtccttgtgtgttctgtgggttcataacacaatggaactcaaatgggcaagactcttgctcttctagcaggagttccaggcctcttgagtggatggggcagcctagaaatcaaatggttgtggcacagaatgtgcttgagtctcagtgtgttggttagtaatttggcaaaactgattgcatggagcacttctgttgaacaggcaattcaagcatttatgtggtcaaggggaagaacaaggctcccaataaacagggtaggaagttgttctaggtccctttgaagctccatggattccctaaaccagcggacctaggcatcaaggactccttgtttctctactcaggcctgaaaccttccgggaccaagaaattacttttcccatggcgagacctgctttccctcctgctggaggaggatctgggggaatttacctctgctctaactcctccctgcagtttccatctgagctctctggtattcactgatattcactggtaggtgaaaggaggcagtggggggaaaggagaaacagggatagcttacacagcagtagggtctcagcctagaaggggcccccaggctggcagccgggttctcccagaacatctaggctgagtgtctctccccctggagaagtacagaagcctggcaccctggtttcacttcagctattaccttcagggatttttttttttttttttgagtaagggaaaagatctcaatgtggctttctgattaattacctcactttttttctggaggtggaaggaaaggattgggagccagcaatactttccctccttttccaggaccagctttgtttaaaggaatcggtgaaatgctgtggtttaaaagaagctccccattgtcaactgctcaccatgacctcactcatcaaaggtgcatcaaaactaggaaacacgcacaccctattgggaaggaatgcgttcgtttcatcagactgttcctagcagggaggacggtaggagagtgtgggtgcctatatgtagactctacaggattgggcaatgtgggtggagagcaggcgtggctgggcctcacagtagggactttgagcagatggcgggggctcctgaggtgcagaagaacagcacacagaaaggaaggaaggagagaatcaacaaatgtaggtttcaccatttttgcctgctttaccatctcctcctctagtaagtcaggccagcaaccatttaattattgtgaggaccctcgcccacattagagtcctctccagtctggcctgatgaggattccaaagcattgaaacctggagaggctccatgatcagctgggagcagcagccagagaaaagggttagggatttcttgtgttctgtggtgccgcacgcatacatcttagatagcactgtggagtctggtgggaagaaaataggatgtgagggagaggagagaggggccaggtggggctctgtctcactaggtcaaaggcattttatcaggcaagctttcccaagaagtgaacagttcctcaaggcaagtccctgtccttattacctgcctgatgggtcagagcagtgaggtgaatgcagcttgtcccccatgtggcttggtactgaagttcttctgagtttacccataagatcgtgcgcctctgtgatggaggacagattccaccttcagccttaccctcagatggagatcaatgtcctagtgatgtgtagagatgagagaatgtttttgtgttagaagttagtgaagtgaaagcacggaagtagcagcttgagatttctctttttgcacgctcgctcactctgaggtttttggcatcaggttaaaggcctacatttaagtactcaggaggctggccgtgctggggttgactttgctggctttgaatctgttcaagtgcctccagttgggtgatctggggagaactgtttaacctccagtgcctccctccgtgcagctgtgaatgttagaggagttaatgtgtgtctaaacatttcttttaggatgtctaaaataaagtgctccttaaacattgtttctcaaatcttccttttttgcctccagagatgtgttgatgacccatgcccacaggacatatatcgccctttgacctctaatgttgccctaattgccacttcctctgtctcctccagaaaacacagatgtagactcagacttccatctggactaataggaggagacaggctgtgaagagtgtgtgtgtgtgtgtgtgtgtgtgtgtgtgtgtgtgtgtgtgtgttggtggtggtgggacaggaaggtggtgcggatgtttaaaaatgttgggaccacgcattcaggacagaatggaagtgtctgggctttggcatccacccacccactctagggtccagactagtctaagaccctcggggttaagaaactctcaaggaagcataggtaattaggtgtcctcttcataatagtagaagacgtcataggttatggtgctggatgagataggtagatcaaaataattccttgagcttctggagtactcgatgtcatcagagttgcccagtaccccactcagttatccctacagaccctggaggattggcgatcgcttaaagtaaaaaactcactctggttgatgcctcagtggctgaggtggaatccatacctcctcccagatgcctggctggtcacagaaatcccaggctttctaagtcagatcttgccaagggtttctctcttctgttttccctttatcccccatcagtgttcctaaacctgaacccccagacccagttcttggggaagtggctgtcatgggcagtgactgtgcaaacctgatgttgcatctccttcctgggctggcgggagtccgaatggaccctctgaacctgtgtctatccttgcaggcagattccccaggcgacggcttcgatgaaggacggcaaatgggagcggaagaaggtgagcagcggccttgactcgccccacctgggctcagggcccggggtccactcatgttgctgacttcagcttctttccttttgcctgtttggttgcagttcatgggaacagagctgaatggaaagaccctgggaattcttggcctgggcaggattgggagagaggtagctacccggatgcagtcctttgggatgaaggtaagatgttgctggaaccctgtgatgtgggactttctgcagcaattttgggaaaggcagcatgtctgggcagaagccagaagctttgttctaggagggtctgaccctctcttggagcccccatctaaataagtgttaaagtcaaggagggagagaacactggcctgctgatctggactcaaaagctggaaatacttggtgggggtcctttagctctctggtgagtgaatagccctgagtcccagtgaaccaggtgttgatggctcttttgagactttggttcctgtcttcttagtttaaaagaatttaaacaagagacacggtgcagcattgaggagtttattgcaaaggaaaaagaatattttgaaagttaagtgcagagtagacagtacacctcgggagagagagaattcagggtgggctgctcataagagtgaggcagtgttggccgggcgcggtggctcacgcctgtaatccagcactttgggaggccgaggcaggcagatcatgaggtcaggagatcaagaccatcctggataacatggtgaaaccgcatcgctactaaaaaaaaaaaaaaaaaaattagccgggcgtggtggcgggcgcctgtagtcccagctacttgggaggctgaggcaggagaatggcgtgaacccgggaggcggagcttgcagtgagccgagatcatgccactgcactccagcctgggcgacagagcgagactccgtcttaaaaaacagaaacaaaaacaaaaacgaatgaggcagtgttgattattgctggagaaactccctttataggagttttacatgattattcataaggaggtgggaagagctgttactagtaagcatgttttgggtggtcctctgggtgcacatgagtagtagctgtacattcttgttcatttggcacatgtcttattagcatcttaaatctccacccaggagtgtgttttttactattataatgagccagggggtcagtttgaggacaggaaaaatcaaagtgcacaggctgtctagatgggaagttccctactgaagatagctttgcttgaatgagctcagttagaatatgaataccgaggcttattgtgttgactataggatcaccaaggttgctgcacttccttgattacctatcctgcctcacagggagggtgcttgtgtcctgcccattctttctttccttcctgcttttgttttgtctgctttcctgtgataatttagaaaacaggaacaagtttatggcctcacagtagagccttacatccattgtccatctgtccttccagtttccctccatatttcagaaaagatttaaaagtgcttgtatgtacactatgatatgattgtagagcctatggtggctggaagacctcacaccggtctcagaaatcacacctaactcctgtcttcccccaggtctctgccttttcctactctgggagctcagtaggcttctggctactcccttagcctgattgcaaccctctggttgccatcagtagcagtgccacccacttgggctaatccaggagaccttgcaaatgagagaggcaattcagctaagagaggggatctgcagggcttcccagataggccaaagagatgaacccactaaggctactatgggacatctctctgttcactttctgttttagggaattacagggattatggggtcaaactgcagccaggaagtctttaaacattaggagatcagttttgacaaccagatcatgtcaaatctagaaaagtttacatagagctgctatggaactctccctttttctacatccctcctaccttcttagagttagaagggatcttaatagattaacccttaattttacattgacatctagaatttgtaagcttatgcctaaggactcatagcaaatggacagtagcctagagcctggtgagcatcccattgtacacattgaatattatcctctccctcctgcctccactccccatcttatttatttaagtcagccagatatctttcctaagcctctgcactgagaagggccaggtgtattcttgatccctggactcctgagagaggcagatgtagcccagcaggatcattgatcatgtaaacaaaccacagccttgccctctgtgcagggatggtgggatgccataagccacaaagagcactccctcctccatcctcttttgtaagggatcaagggtcaaggtttagtccatggagaattttgatataaaaggcagaggtcaggagttaagtaaaactaagagcactcatagccaaataggtgagcatttcattgatgaagcagtaactgggagacaggctccagaccagtggccgttccaaggctcctgcccccttccccagggggtcctcttccccatgactccccttccccaggcttccttctccgtggcaccactacacatcaatattcctggcaatattcttcatcatggagacttcggcagcgacttcaaccagatgaaaagccccacctcattcctgaatgattgaggtgctaggtaagcttgtctgtccatctggcactgcccaactctctaccctgggatgcttccaagaggtgcatgggtacctgggaccttgagcatcagtgtcaggaaatggctgacacctctcgtgcatgtgtttgattgaaagagatgtctacggcctgctcatttcagtgttaatctgtgcctcagagcaggtcacccactgaatgttttattcccattcccagcatggtgtggagggtgccagtgccttcactttgcatttcttctgtccagatgtctctggagggtcattcttaactgtcttgcttccagaacatgctctttgtaaacccctgaaacatggcttggatcattccgtctcccacctcagcccctccggagctgcctggacctcatcattccggagagtctaagtggcctcttctcgtgctcagctgcttacctcaaccacgtcattatcatacttttgagtctggatgaattttgtcttagggcggagagctcatcagttctaccggtcttggagaacaagattttctaaagggaaaggaagattcttgaacatattgtgaaagagatgtataagcacccagacaggaaatgaggataagtgtttattaaacacaagttatttcatactagcctcatcctctttttggaaaccattattaaactggtagagtatggaaatccctagatgtcagatacccggactatttaaagttatgttttaatcatgtaataaaggaatgcattctcatataaattaaaacttggacaaggctatacttgattaccatctgaaccctagtctcctcacggtaaacatcagtttagcatgtcatccgagacttttctatgcatttgcacagaatgcatacagttccatgcctgtggatggattctttggctgcaaacaatagaatattgtttgtgatgttaagaaaaaaacatgtttatctcagaacaactaaatctagcatatttttaaacagttgtgtggaattttattgtgcagataaattgctttttatttggccattctctcaatgatgggtatttagattgtgtatagatatggactttggtaagacatctttcatgattccctcggggttcggatggagccccttgagcttggtgctattgaagggattagtgcttggtgctgttgtggaatgggccacctcagtttggagggagatagtatgcaatagtgctccaccttgcccaccattctcatcaacagcctgaaccatgtgtcatttgtggatggcaggaagcaaggagaggtagttaatacttaagggtgcagaatcaggtccaaaaagacatatgaaggctagaatggttgggcaccctcacaggaaatttagctagaatacatttctgcaccctacttgtgggtccaaaaagcagctagacaagttttagatgtagaagcatttgtcaaaagcttttctgggggagcagggctttggtttacaaaagagcagtgtggttgtcagtggcatagctgatagaaaaggtaacctgacatttggcagcattaaaagaagtatggcttccggagcaagggaggtgacattcctcacactgtggcctgacctgcagcctacctgtggtctgagtcatacaggtcttaacgctctatactgttgtgtctgcgagagactttttctcaatagtgttgctgaatgacaggtcgtgacctcagagactcagctcattcatgctggtgtgttccttagaatgtgttaagaggcacaaaccaatcaagaagcgagccttgtgctttatgatcaaggttttccttgagggaacagacaagcatctcttaacagtttctttgtagccagcgttgtgatgaatccaactttagcccagagggttggatgttgaacctgaggcaccagttatcaccgattttttcaggtccagctaatgtttgctgaggtctactgtgtcggttcctgtgcttgaaatgactctgcagggtcagaattattatcccaggtttacagataaggaaactgaggcccaggaaggttctatgatatgctgaagataatgcaactatgaactggtgaatttcggctcagccttccaggctggcattctttgcccagtaccacttaactttattcatgatccattttaggtaaacagtccctgtgccatgccggtatgcttagctattccaaagacagatactctccaggatgggcagctcctatctcagtgtgttacctggaatgcttactgcccagagcagtgattctcaaactcaagagcctcgggttctggcaaggtgccttagggacacaggtgggagcaaggaggggcccagggagacagcaggtggtggcacattgagcctttacctctgcagctttgaattgacctatttttatatttgggtctctgtttaagatttcattgccgtggtagggagagttccaatgctaaactaaaaattcagctgtggggcagagagagcacgtgacggtgagggttcttcttcagagcctctttaactgggaggcctccttgctctcctctttgctctgctgagccagcaaggccctgcagccagcataccagcctctcagggctgtcttctgtgacctcaggcaggtaggagccaccagctgccctggagtcctgcttagaaagtgaggcttcctctgttgcctcaacatcaggagtctgccctggggctgggattgaggtgagggaaccccttcatgtttctccccaaggcttctctccgggagtagtctttgaatggtgggtacccagaggtgctccaatgtctgtatattaggacatctgaccctttccaggacacaccaggaccctgacaggcacgtgtgtcctgggcctgttgtcaccctttccatcccctcagtgttgtctcctagtgtcggaaccccaacccccagctgagttcagctcttctctgccctggttatctcaaatggaaaatgagaacagaggtttgctcagccagattccttccctgcagtcatctgcagataaatgagatactctctgtgaaagaaggaagctgagtcagtgcttcgctcacccctgaggaagatgctcacctgctcctctccccaaccccctccctccagccccagtcacctgtctgtggagggggccaggttcagcctgcagggtccttcctcctgccagcccccaggttgttcttggctcctgagcatggagtagacgtcagtcaggctggaccttgtgatctctattggtgttcccttcctctccccactccatatgagaaaaaaaaatacatctggattagagatgaacagggccattgttctggccggcctcagggccagcagacgaagaagacttgtgacaacgatggcagggatcccatctcctcgcctggacactttctcctccccaccagaaatgagtaggttacagccgaggggagcagcagtgtctgaaaaagaccaccagcgttctgagctggaggttcttatagggcgtgggagaggcagcatctttccccaaaacctgcctgatggttggtgtggggccacttggctttccttattccacaaggctaatgatgaaaaaagggatccagaggcttcaccttaatcataaccattttagcttctgccctagaattactgaatttgaagtcctctttcctattacgttcttttttattttgttttatttttttttttttttctgagacatcttgttctgtcacccaggctggagtgcagtggcacaatcttggctcactgcagcctcaacctcctaggcccaagtgatcctcctgcctcagccttctgggtggcacaggtgcatgccaccaaacttggctagttttttaaattttttgtaggggggggggtctgactatgtttctccagctagtctctgactcctggggtcaaatgatcctcctgcctcagcctcccaaagtgctgggattataggtgtgagcaatggcacccatctttcccattatattctaaagtatctatactgtatccctaatttcaaaagtaatttatgaataatgtagtagaaaacttgaaagcacaggaaagcaaaaaggagaaaaataattccactacccagagacaaccgctgttaaagtactatgctgtgtattctttgtttttgtctaatcctgggctcactctttacattctgtctcggggttaagggggatttctcttcaaataattcccagtgattgaatgtgagagtgagagatggctgcaatcatctgcccagctcctttatttagggttgaggacccaggtccaggggaactgacctgtacctttccacacagtgggcagtgactgggggaggacacaggtcaggcttcctgatttagagggcatgcagtcttgctcccactctgggtcctgccttgtacccctgttgtcctcctccttcgcttctgctcactctgttttagtacctgatgcacctgtttcctggactggaccctccagctgcagctgccatttctccttctccctttcctggctgtccgctccctacatgactgccctccctccctcaggactccctgctttctaaggacttccaagctccaccttgtccagacctatatgagctattgttgcatctgtgtgtgatcacagcatggaggcagacgcaccttcttttgagctctggctgtgcccctcctcattgtggaagccctgcttccctcagctctcaagcagacaagaacagtcccagcatcattgtgtggtcatgagaattaattaagctgatcatggtacttagtatatggtaaatagtacttagtatgtggaacatggtacttagtgtatggtaaattaactggagaattaattaagctgagcatggtagttagtatatggtaaatgctcaaaaaatgtttgccattcttaataataacaatactaataattaagaatgacacttcctccctctctcttgcttccaaccagactatagggtatgaccccatcatttccccagaggtctcggcctcctttggtgttcagcagctgcccctggaggagatctggcctctctgtgatttcatcactgtgcacactcctctcctgccctccacgacaggtaggtgtgtccttacattgtggattggtcacagaagccacagaccagttaacaaatgggccctccatctgggccttccccagacagtggtaaccagctgtggggagaggtccacctgggtcctgcagaggctggtgttttgttagacacccctacgttggataggggagggtgagccagctagaagtgcttggggtctaggtaagctgggcacagggacaaccagtgacccccatggatgctttctgcagcccctgcagggctttctgattcccagacccacttgaaacagtaatatctcaagaatttctaagatgtttctaaactgagtctggcccagatccataacagggactcctgccccagagcatctgaggaggaagagatgagagcaaagaggctgccgtccagcaggagagaggctctgggaaagagctggctcaaggaaagggaagacctctggaagccagggatgagcgtggggatcctggtgctgccccagcaggaagatgcttcgctttcttccaggcttgctgaatgacaacacctttgcccagtgcaagaagggggtgcgtgtggtgaactgtgcccgtggagggatcgtggacgaaggcgccctgctccgggccctgcagtctggccagtgtgccggggctgcactggacgtgtttacggaagtaagtgcctggcagcctcagcgtcaggaggacgggagagatagggagcagagaggcccatggcagggaaagcctggcgttttacagaaagcctctagcttattgtctctttcatccatggtaaaaggagaaaactgtgttgccaaaaacacacctttgtggtttgaggagtccttgcggagcctggtatggaggcagctatgtggttttctgcaaaactcgggctttaaaggggacccagttcctggttccgggcttctcagactgctaacatattgacaaagataaagccacaaatcagttctgaagttgagggtctggagcaggaagagagggggctttagtgtatagttgagggaccctgatggaaatgcagtcccattgccaagaaaatgtccttttcagtgtccatgggcattctttttcccttcttctcttgctgggtggacagaagcccgaggatactggcagtattccctcagggtcctcggggtgggctgctttcccagctggagcagaggctcagcctcacaccagatccagtagggaagagtgcatttgcactcgaatccttttggctcctctcctgaaagatcctgtccccaacattggagcttcccctggtgaggaaggggaaaggacatggactttggattaggtagttgtggttcgcaggttactagctgtgtgtccttggaaggtcactggccctctctgagcccctgtagcactcactgtgtactcccagcccagggcacacagtgccttttggtttgtaaccttggtcatctttctcagctcttagatcataagctccctgaaggcagggcaagccttgtccatctttgtatcaccatcctccctaacacagcaccttctcctgctctcttcagcatccttgaaccacttgctggtggtacttacatggttacatgtggccgggactcacaggattacatggttacatgtggccaggactcacagggttaaatggtcaaaattcaatgggaccaacagagccctgaggagaatggccggaagatgcctaaataacatacccaagggcaatcttctcagtgagcagaagggtggaaaagacactggagggtggggtggaggtgccacagatgggcaacctgtcctgatgagagccaagatgcagttacctgcgtggagtgtcttggaagagcaggacactgtgctaaaagtccagcaaggcacatcgacagcaagtgctgcatggatgcaggggggtgagtttcggggaaaagcccagtaacaacaacaacagtagctcctattggtcaagggcctcccatgttaaaggtgcataatccccacagtaatgttatgagggagataggatcaacccatttcacagataaagaaactgaggcttagatcaaggttcacccccaaggcacctaggtggttaagtggtgaaggtgagatttgaatctggttcacactttgtgctgtgtccactctgctgagatggaacattcatgtggaagcccattattgagtccagtgaccaccctgacaaaatagaagtggaaggcaccagaactcctgactgccttccctccaactttcctgttgcctggggtggcccaagagggtgtggccagtccatggcagccaacttagaggtatctctttctgggcaggagccgccacgggaccgggccttggtggaccatgagaatgtcatcagctgtccccacctgggtgccagcaccaaggaggctcagagccgctgtggggaggaaattgctgttcagttcgtggacatggtgaaggggaaatctctcacgggggttgtaagtatcaccacctggggctgggggccaggagtcagagggaggagaggaaggaaggcatcttgtaggggctggtggcagcgtgggtgaatagattcagccctgggagctgaagataagggaaatctgcttgagtcagcactctccggagcaggtgggcgggagcctcccgtctccagccttgatagcagaggccttggcagcagagagcccggctcaggcctgttatatcgtagtcttgctgcagagattgtggcccttcccaggcccagcctctagagaaaggctcctttgttctccacatgccgtgggagtgaaggagtgctgcttgggtgccagctggacgcagccgcagcaggtggggatgtggttggggacggccatgtagaaatttgcaccctgtaagctccccagaccctgccttgacagcctgccctacctactcccaaatgagccctctgtgctggctgacccccttgctttccccaaatcaaggcataagacccccacttcttgtctttgcttccatcaagcccttcctggcatgtgcgtctcctacgagcttaacctgacttacacttcaagtcctgtctcattcattcagtctgtggatattccttaagtttcactgggtaccagacattgtcatagctcttgggagacacgggtgcatgagggaggcatagctctccttccaggggcttgctgcctccttctgaagtcttgcttggcctttccagccccttctcaattctgagaaccatgttctttcttgttattaaggttcagttccatggggtgtttttttttttccctaatctttctacttaacctaagttctaagttccctgaggacaaaaaaacatgacttaagcctctctgcagcttgtgtgggtgggcccaggccatggagtcaaagggtcaggaaaatgggctgggtgttcttggttgtcctggctgaccctcagctgggtgattttcgctggtgaggacagcactgtggcaggagagacggggattttggttctgccctcccacctctggattgagagcccagccctccaggcctctctgccccctccatcctgaggaaagaagggtgcctcctgctgcccagcagccccacacagtccatggaagtcagcagggctatgaccagcagcatgcgaggaggtcagcagagactctgacctgtctgcatcctctgtcctctatgctgtgtgggctcctcagggcagagcacacttcactcatcttgcacctggtcggcctctgagcaggttcgttcctcccaggagatgctgctcgtttcccaggctgaggtttgagctcatcaccattgccagccaatctgggcttcagggttttaccctttcagccttctcagaaagcagctgtctgccttccccatcgcagccttgcaatttattgccattaccattaggtagcagtgacattccagagcttttcctgaaagggactcctgaatataagctctggcagagcgagggggtgggggagggagggggaccttgcagagagatggggaggagggggtgagaggtgtatgggctctgccctgctgggtgtatctgctgcaggacacagagttccatcaaatggaccacacagtgtccccatcttaggaggtgaaacctcttggtcaaaataactacccttagcaaattgaactgttcacccacatcagcaaatagtcttagagtagccatttggaaaaagagacatttttgtcacataagaaatatattttcctaattcttccctgcattttccccagttcaattctgttctaacctcagacacagaataacattagggtcaggttggtgtcccaaagtgcctgactcctccctggaattctcccacctgtccaccagggaagactgagaatcctccttttacttgggagccctgtgatggacacctccccgggcttgggctctgcaggcccacacagaggacagagagatgtgccgaggtgcctgcgttatgggccctcttagttggactttcctctgctgctgcaggccctcagccccggcagtggcagcatggtgctccagatctcctcccagcagctagctgctccaccccaccacccttcttgtctgtgactccttggagaggatccaggagccatgcagcaagaagcctgcagacctgtcacctcccacactggagaggctcccgtgaagcccggcctcagcagtcatttcccataccatgattctttctctgtttaaaaaaaaaaaaattcttccaatgatatctttatgaaaacagaaagagaagcatctgtgtttccttctaattacatcttgtagctgcctgtttgatttgcatctttctaagatggttgactttacaagttatctcaataaaagtggccagatgcctaactcagaaccagagcattttgaaccacaaaatggaattgaaattttggcccagaacaagctggttctgttatcaggcccctgggtggggcagggggcagccagccaggtcctagacataacttttgggggatatggggcttgtgtcccctcagtgtcacaacatgcctcacagtggacttcgcatgcgttgatatttgaagcacgatcatcaaaactttgtgataattgatcgtagtgtttagtaacaatgtaaacacttaaaaaaattcaagatagaaaataaaaatgaaggcaagttgggactgccagagaagacccgtcactcctcatccaagttatctgcgactcccatatgttttgtgtcaaagactcacctttattgtgctgtccaatcccttccccagtgcagaaacaagtctcccatggagggggctggggcagacacagtttgctgaaaggagcaattttgagtggttgtggcattctgtgtccatttctggctccacagctttcttcatttgtaggaacaagtccttgtcctgttgttagtggctgatggaagttgtcacccaccaggcaccaaggcaggagtgaccctatactgtctttcttgtggagctgggtcttggcagccagatcttgattcaggatctgccatgcctcttcctgaccacagccccgctcctccatcctctgcaggtgaatgcccaggcccttaccagtgccttctctccacacaccaagccttggattggtctggcagaagctctggggacactgatgcgagcctgggctgggtcccccaaagggaccatccaggtgataacacagggtgagctggggaccttgcagagggagggggaggaggggatgagggagtgtgggatctgccctgctgggtgtatttgctgcaggacacagggttagtgagaggcagtgagggtgccttggaccctgccctgagtatagctcccttactactggtgggagggtggtaaagggaggggttaaaaaaagtcgttggaaagatgtactgaatattcataaatcatgtttatgtagcatttttaagacctagatattttgggtgcaagagaaacctctacgagagagagagtttcatgcgagaggtcgtaggatggctggagggaggccctaattaagcaacaggatgctggattctggtttttggccctgtctcttccctgctgtgtgactctcccttctgaggcttggattcttcacttgtaaagtgagagttaggggcagatgagccaggagcggtgaggacactttgtgctctgtaacttactaaggtggtaccttgggccgtctgacagccctcgagagagagtttgctcagccgtggagtgggaatgagaacagtgccctctgaccacccctcgggctgctgagtgggctgtggccacctttgcagtggatccagtactgtgcggatgtggttgagtgaccaaggagggctccagtttccttaaccctgtagacgtgtatctttccccatggactcttgggtgtctcacagttgggcagagattgcagggagagggcagctaaaccctaggcattgacaataccagggaagggcagagccagggggtcccactggcctgggtgtccaagcgccgaaggaaacaaggcagggagctgggcagcagttgcttcggttacttctttctgctttgatttcctgaggctggcaaggctctgagtgccaggaaggggtaagagtagggaattttcctgtgtcccctgggagaataataggtaggccaggatggccaagccaggtgcagggttggggagcggagtggagttgagctgagttttggagaggagtttctcagggtggttaaaacatcctgggtttcctggcttgggcccagattatgcctcttctgagcccactgaagggcgagtggacctgcctggagcaagctgcctttggggtccccagggcagcgaggagcccatagtccagagctaggtgccagtggcttcctgccccctcctgtagtgctcaacaaacagtgacctcatggtagcttctctctgtccccaggaacatccctgaagaatgctgggaactgcctaagccccgcagtcattgtcggcctcctgaaagaggcttccaagcaggcggatgtgaacttggtgaacgctaagctgctggtgaaagaggctggcctcaatgtgcgcccctctcccccacgctgcctccccatccctgtcagcactagtcttctcccccacatttccagagcccgttctctgagcggaggcctaggtcccagccttgcatcggcctgtctacctgtgaggggtagctgcagtttcttcaactgcaaaatgaagatactgcctggccccgagtgttgctaatggcactgctttgtgtatgagtgctgtgggaatggaggcagtagaagtgtccccatttcacagccaaagaaaatgacgagctagtgtgtttgactctgcccgacatggctgccaggccatgtttgactctgcctaactcccctcagggctcctcatgccgtagcacccgggttcttgattcacttgcaagctctaggagccctgctgccttgcacggcttcccgttggcgccttcccctctggttccctgtttagatcaaagtctgtttcaaagcctgttgctcagccagtgggagctggcagaagggataggcagtagagctgccatgtcctcacccctctgctcccctccactcctgcatgccagtcatgccactgatgccgtgcaggaggctgtgtcagagcaggaggggccagagtggagtctcctcacagccctgcctccctgcttttctttcctccctgttttcctccaagccttcgcctgtgcctggcagatctctttgccctccctttaaggagatcattggctgttccaggaagctgatgccgaagggcacacagcttggcccatttgccctctcccttctggtcctgaattactgagcacattatccaggctggagccctacatcctaccaatgggtgatttggccaagagaggagggtggacgtggtgcagccaggaggtgtaacagtcaccttgccttctccacacaggtcaccacctcccacagccctgctgcaccaggggagcaaggcttcggggaatgcctcctggccgtggccctggcaggcgccccttaccaggctgtgggcttggtccaaggcactacgcctgtactgcaggggctcaatggagctgtcttcaggccagaagtgcctctccgcagggacctgcccctgctcctattccggactcagacctctgaccctgcaatgctgcctaccatgattggtgaggagggccctgtagggctggctggtgtccttgaggctggggtggggtctgccctggaattgaactctacccaccttcctttagcccctcttcatgtcccagggtgtctctggatctgcaccatacagagggtctgatgccagttttcagaaccttcagggagtggatactcagttcaaagagggaaagtgccttatccagggtcacagagcagcatggcaggggtggggccatagcctctattcctgcccagctgtggatcctcagcttgccatgttaggtacactggaccagcttgtggagccatagcccaggagctcagggacattgagtgcaggtttcttactcctacctgctggccctgtggctgtccctggtggccagcccagctgcagcaaaacctacaaagcctccagccatggtaggcgtcttggacctgccccagtcagctggggcttgggctgctaggggttttggcacacgtccatgtttggcggagggtgtgccttcaaaccctgaagggcctaatttcaccattctttctggctgcccaagggaacttccctgcttttctcccttgctgttggctggataaaactggcaatcagaaagtcaagagctacagctgatggtcatggtgttcccagagagtcaggaatatccatggaagctgagcagatgccctgttgctctcccatctcagctctttgattctgagaccatcatccgctcattgcacctttgatcacaaaagctttgaacttctgattctgctcccaatccctcgctcctttttcccctatcccctgtgccaaccaggagtttcttctatttccaggcctcctggcagaggcaggcgtgcggctgctgtcctaccagacttcactggtgtcagatggggagacctggcacgtcatgggcatctcctccttgctgcccagcctggaagcgtggaagcagcatgtgactgaagccttccagttccacttctaaccttggagctcactggtccctgcctctggggcttttctgaagaaacccacccactgtgatcaatagggagagaaaatccacattcttgggctgaacgcgggcctctgacactgcttacactgcactctgaccctgtagtacagcaataaccgtctaataaagagcctacccccaactccttctgcacttttgtgtggtcattatcctaaagcgccaccagagggcgtccaaaggcagacgtagggtttggtttagactgcgggagcggagcgggtgtgggggaagatggggatgagcaaatggcttggttgagttctttgaaggtgatccctctcttgtctgccgaaggttactcagaggcacttttacaggagcaaagctcaatgtatttcacagtgctacggtatttcagaccccttccatctgggaatatacatgcacgttaataagtaagattcaacacacaagcccagcattatgtaccaggcactgggctaggtgctttactttaagaggagaattcaaacctgaccctttttccatagaagtctggtgggagggacagagcatgcagaggtgactggaagcagtgagtgatgctacaacagaggtgtataggaaattctctagagtccaaaggaggaagtaatttgagctgaggatattggtgtctcaggagatgtttagaaggatttcataagggaaagaaagtgtaaaattatgggggtatgagactatttggcaagttcacaaagcaaggtcagtgtgggttggtgaacatgtcacttttttttgaaagatgattttcatgcaaaatacagctgaacggcactctacccaaaagattcacttcagggaatttttttccccattaataagggatgatccattcttactgtgtgcccagtagtgtctgtttctgagattcctggcctggctaagaaggcttgacacagggcagctgcttggcactggcaggggcgagtccagcactctggcctccttagttttgtgatggagctaagcatcaataagaactccagcagtaagggctgtgttagtgtctgcagtgaactcagaggggttggccttgctgtagctcaccttgaagggaatgggcctggttgtgaatgtcatggtcaactctggacagctgtctgactgggaactcagtgtttaattatcctagacctttgtttcctcatctgtgggagggctaatggtgcctctcacagaactgcaatgagaatttactggattaaacagtgacttacgggaagtgccaggagcacttggtgaatgttggtttcttcatcctatgttagtctgagagcagacaggcaggtctcaattctttacatagaaccaaccccatgaaccaaatagttctcaacatgacctcattgcaattatgttcagccagatctagacactgggtgtcctgacatcagacaacccattctctccaactggaaatatacctgtgcctcacatggcatccactgaggtcacttgagtgcactgatggatagaaaaacaggctgaaatttatgaagttaaaaattcagttaaaaattgagctcaatgtttagcctctcagcctccttcctcataagcccctaaacaaatcatgttctgtgcactaaggtcttcggaacagtactagaaacgcagattacttgagcatctcaaaatatcttcctagactgggtcatgaaagagggcatgggacgatcttatcgtatcacgtctcccatggctgtccacatgacctctcccaaacttaaagggcaggattcgttgtaaaattcagctggtttctttaggaaactctgtaatatttttcataatagctgcatcaatttacattcccatcaacagtctagaaagtttctccttttctccacatctttaccaacacttgctatctcttgtctttttggtaatagccatcttaacaggtgtgaggtggtatctcaccgtggttttgatttgcatttccctgatgactaagtaaataggccagacacaaaaagaaaattattgcacttactcatttatatgtggaatccccccccaaaaaagaggtcaaatatattgacataggaactagaaaagtagttgaggggggtgtctagggagatacaggtcaaagaatgtaaagtagaaaatacatagggtgagta

SEQ ID NO: 8 - DNA, 57 bp, Artificial Sequence
  Synthetic construct
ccggaggtgataacacagggaacatctcgagatgttccctgtgttatcacctttttt

SEQ ID NO: 9 - DNA, 21 bp, Artificial sequence
  synthetic construct
aggtgataacacagggaacat

SEQ ID NO: 10 - DNA, 21 bp, Artificial sequence
  synthetic construct
atgttccctgtgttatcacct

(19) United States
(12) Patent Application Publication
(10) Pub. No.: US 2013/0203618 A1
(43) Pub. Date: Aug. 8, 2013

(54) METHODS AND COMPOSITIONS FOR THE DIAGNOSIS AND TREATMENT OF CELLULAR PROLIFERATIVE DISORDERS

(71) Applicants: Lewis C. Cantley, Cambridge, MA (US); Matthew Vander Heiden, Belmont, MA (US); Jason Locasale, Ithaca, NY (US); Hadar Sharfi, San Jose, CA (US)

(72) Inventors: Lewis C. Cantley, Cambridge, MA (US); Matthew Vander Heiden, Belmont, MA (US); Jason Locasale, Ithaca, NY (US); Hadar Sharfi, San Jose, CA (US)

(73) Assignee: BETH ISRAEL DEACONESS MEDICAL CENTER INC, BOSTON, MA (US)

(21) Appl. No.: 13/699,922

(22) PCT Filed: May 26, 2011
(86) PCT No.: PCT/US11/38208

§ 371 Date: Apr. 17, 2013

Related U.S. Application Data
(60) Provisional application No. 61/348,527, filed on May 26, 2010.

(51) Int. Cl. C12Q 1/68

(52) U.S. Cl. CPC C12Q 1/6886
USPC 506/9; 435/6.11; 435/26

(57) ABSTRACT

The present invention features methods and compositions for the diagnosis, prognosis, treatment, and/or amelioration of cellular proliferative disorders utilizing enzymes of the serine biosynthetic pathway (e.g., phosphoglycerate dehydrogenase (PHGDH), phosphoserine aminotransferase (PSAT), or phosphoserine phosphatase (PSPH)).

### CROSS-REFERENCE TO RELATED APPLICATIONS

This application claims benefit of U.S. Provisional Application No. 61/348,527, filed May 26, 2010, which is hereby incorporated by reference.

### STATEMENT AS TO FEDERALLY SPONSORED RESEARCH

This invention was made with government support under grant number NIH 5 T32 CA009361-28 awarded by the National Institutes of Health. The government has certain rights in the invention.

### BACKGROUND OF THE INVENTION

In general, the invention relates to methods and compositions for the diagnosis and treatment of cellular proliferative disorders.

Cancer cells rely primarily on glycolysis for glucose metabolism. This phenomenon of altered metabolism in cancer cells, known as the Warburg effect, is characterized by increased glycolysis and decreased oxidative phosphorylation. The M2 isoform of the rate-limiting glycolytic enzyme, pyruvate kinase, is expressed in cancer cells. In contrast to most adult tissues that express the M1 isoform, cancer cells exclusively express the M2 isoform of pyruvate kinase (PK-M2). PK-M2 is necessary for establishing the unique metabolism of cancer cells. In addition, the enzymatic activity of PK-M2 is regulated by tyrosine kinase-dependent growth signals. Regulation of PK-M2 activity by tyrosine-phosphorylated proteins alters metabolism in a manner that helps satisfy the distinct metabolic needs of proliferating cells.

Because tumor cells exhibit increased glycolysis, it is surprising that phosphotyrosine-based growth signals cause a decrease in pyruvate kinase activity. The decreased PK-M2 activity associated with cell proliferation may reveal a novel role for an upstream metabolite in glycolysis to signal energy status or to allow flux through an uncharacterized metabolic pathway.

There exists a need in the art for methods and compositions for diagnosing and treating cellular proliferative disorders.

### SUMMARY OF THE INVENTION

The present invention features methods and compositions for the diagnosis, prognosis, treatment, and/or amelioration of cellular proliferative disorders utilizing enzymes of the serine biosynthetic pathway (e.g., phosphoglycerate dehydrogenase (PHGDH), phosphoserine aminotransferase (PSAT), or phosphoserine phosphatase (PSPH)).

We show that diverting carbon from glycolysis into the serine biosynthetic pathway produces NADPH. In particular, we found that RNA interference-mediated knockdown of an enzyme involved in the serine biosynthetic pathway, phosphoglycerate dehydrogenase (PHGDH), significantly inhibited the production of NADPH and the growth of cancer cells.

In a first aspect, the invention features the use of a phosphoglycerate dehydrogenase (PHGDH) gene copy number in a biological sample in a method for diagnosing a cellular proliferative disorder in a subject or assigning a prognostic risk of developing a cellular proliferative disorder in a subject. The method includes obtaining a biological sample from a subject, determining a PHGDH gene copy number in the biological sample, and comparing the PHGDH gene copy number in the biological sample to a control gene copy number, wherein an amplification of the PHGDH gene in the biological sample relative to the control indicates the presence of a cellular proliferative disorder in the subject or the risk of developing a cellular proliferative disorder. In certain embodiments, PHGDH copy number is increased by at least 3-fold. In some embodiments, PHGDH gene copy number is determined by hybridization-assays and/or amplification-based assays (e.g., fluorescence in situ hybridization (FISH), comparative genomic hybridization (CGH), or microarray-based CGH).

In a second aspect, the invention features a method for diagnosing a cellular proliferative disorder in a subject or assigning a prognostic risk of developing a cellular proliferative disorder in a subject. The method includes obtaining a biological sample from a subject, determining a PHGDH gene copy number in the biological sample, and comparing the PHGDH gene copy number in the biological sample to a control gene copy number, wherein an amplification of the PHGDH gene in the biological sample relative to the control indicates the presence of a cellular proliferative disorder in the subject or the risk of developing a cellular proliferative disorder. In certain embodiments, PHGDH copy number is increased by at least 3-fold. In some embodiments, PHGDH gene copy number is determined by hybridization-assays and/or amplification-based assays (e.g., fluorescence in situ hybridization (FISH), comparative genomic hybridization (CGH), or microarray-based CGH).

In a third aspect, the invention features a method of identifying an inhibitor of PHGDH. The method includes contacting a cell that expresses PHGDH with a candidate compound, determining the level of NADPH in the cell, and comparing the level of NADPH in the cell contacted with a candidate compound with the level of NADPH in a control cell not contacted with the candidate compound, wherein a reduction in the level of NADPH in the cell contacted with the candidate compound compared to the control cell identifies the candidate compound as an inhibitor of PHGDH. In some embodiments, the cell is provided with an excess of phosphoserine aminotransferase (or a functional fragment thereof) and/or glutamate.

In a fourth aspect, the invention features a method of identifying an inhibitor of PHGDH in vitro. The method includes contacting a sample that includes PHGDH or a functional fragment thereof and NADP⁺ with a candidate compound, determining the level of NADPH in the sample contacted with the candidate compound, and comparing the level of NADPH in the sample contacted with a candidate compound with the level of NADPH in a control sample not contacted with the candidate compound, wherein a reduction in the level of NADPH in the sample contacted with the candidate compound compared to the control sample identifies the candidate compound as an inhibitor of PHGDH. The sample contacted with a candidate compound may also include phosphoserine aminotransferase (or a functional fragment thereof) and/or glutamate.

In the third and/or fourth aspect, the determining step may be performed using fluorescence spectroscopy.

In a fifth aspect, the invention features a method of treating or reducing the likelihood of developing a cellular proliferative disorder in a subject in need thereof, said method comprising administering to said subject a therapeutically effective amount of an inhibitor of phosphoglycerate dehydrogenase (PHGDH). The subject in need of treating or reducing the likelihood of developing a cellular proliferative disorder may carry an amplification of the PHGDH gene. An inhibitor of PHGDH reduces or inhibits the activity or expression levels of a PHGDH polypeptide or nucleic acid molecule. The activity of the PHGDH polypeptide inhibited by a PHGDH inhibitor is the catalysis of 3-phosphoglycerate to 3-phosphohydroxypyruvate; conversion of NADP⁺ to NADPH; or promotion of cell proliferation. Examples of the inhibitors of PHGDH are, e.g., peptides, nucleic acid molecules, aptamers, small molecules, and polysaccharides. The inhibitors of PHGDH may also be a short interfering RNA (siRNA) or microRNA.

In a sixth aspect, the invention features any one of the methods described in the fourth aspect, further comprising administering to said subject an additional therapeutic agent. Examples of such additional therapeutic agent are chemotherapeutic agents.

In a seventh aspect, the invention features the use of an inhibitor of PHGDH for treating or reducing the likelihood of developing a cellular proliferative disorder in a subject in need thereof, where the use includes administering to said subject a therapeutically effective amount of an inhibitor of PHGDH.

In an eighth aspect, the invention features the use of an inhibitor of PHGDH for treating or reducing the likelihood of developing a cellular proliferative disorder characterized by an amplification of a PHGDH gene, where the use includes administering to a subject in need thereof a therapeutically effective amount of an inhibitor of PHGDH.

In some embodiments of the seventh and eight aspects of the invention, the activity of the PHGDH polypeptide inhibited by a PHGDH inhibitor is the catalysis of 3-phosphoglycerate to 3-phosphohydroxypyruvate; conversion of NADP to NADPH; or promotion of cell proliferation. Examples of the inhibitors of PHGDH are, e.g., peptides, nucleic acid molecules, aptamers, small molecules, and polysaccharides. The inhibitors of PHGDH may also be a short interfering RNA (siRNA) or microRNA.

In any of the aspects of the invention, the cellular proliferative disorder may be cancer (e.g., prostate cancer, squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, vulval cancer, thyroid cancer, hepatic carcinoma, gastric cancer, melanoma, or neck cancer).

By “amplification” or “amplified” is meant the duplication, multiplication, or multiple expression of a gene or nucleic acid encoding a polypeptide, in vivo or in vitro, and refer to a process by which multiple copies of a gene or gene fragment are formed in a particular cell or cell line. The amount of messenger RNA (mRNA) produced, i.e., the level of gene expression, may also increase in proportion to the number of copies made of the particular gene. A PHGDH gene is said to be “amplified” if the genomic copy number of the PHGDH gene is higher than the control gene copy number, which is typically two copies per cell. In one example, a PHGDH gene is said to be “amplified” if the genomic copy number of the PHGDH gene is increased by at least 2- (i.e., 6 copies), 3—(i.e., 8 copies), 4-, 5-, 6-, 7-, 8-, 9-, 10-, 15-, 20-, 25-, 30-, 35-, 40-, 45-, or 50-fold in a test sample relative to a control sample. In another example, a PHGDH gene is said to be “amplified” if the genomic copy number of the PHGDH gene per cell is 3, 4, 5, 6, 7, 8, 9, 10, 11, 12, 13, 14, 15, 16, 17, 18, 19, 20, 21, 22, 23, 24, 25, 26, 27, 28, 29, 30, and the like.

By “biological sample” or “sample” is meant solid and fluid samples. Biological samples may include cells, protein or membrane extracts of cells, tumors, or blood or biological fluids including, e.g., ascites fluid or brain fluid (e.g., cerebrospinal fluid (CSF)). Examples of solid biological samples include samples taken from feces, the rectum, central nervous system, bone, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Examples of biological fluid samples include samples taken from the blood, serum, CSF, semen, prostate fluid, seminal fluid, urine, saliva, sputum, mucus, bone marrow, lymph, and tears. Samples may be obtained by standard methods including, e.g., venous puncture and surgical biopsy. In certain embodiments, the biological sample is a breast, lung, colon, or prostate tissue sample obtained by needle biopsy.

By “cancer” and “cancerous” is meant the physiological condition in mammals that is typically characterized by abnormal cell growth. Included in this definition are benign and malignant cancers, as well as dormant tumors or micro-metastases. Examples of cancer include, but are not limited to, carcinoma, lymphoma, blastoma, sarcoma, and leukemia. More particular examples of such cancers include, e.g., prostate cancer, squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, vulval cancer, thyroid cancer, hepatic carcinoma, gastric cancer, melanoma, and various types of head and neck cancer.

By “candidate compound” is meant a chemical, either naturally occurring or artificially derived. Candidate compounds may include, for example, peptides, polypeptides, synthetic organic molecules, naturally occurring organic molecules, nucleic acid molecules, peptide nucleic acid molecules, and components and derivatives thereof. Compounds useful in the invention include those described herein in any of their pharmaceutically acceptable forms, including isomers, such as diastereomers and enantiomers, salts, esters, solvates, and polymorphs thereof, as well as racemic mixtures and pure isomers of the compounds described herein.

By “cellular proliferation disorder” is meant a disorder associated with abnormal cell growth. Exemplary cell proliferative disorders include cancer (e.g., benign and malignant), obesity, benign prostatic hyperplasia, psoriasis, abnormal keratinization, lymphoproliferative disorders, rheumatoid arthritis, arteriosclerosis, restenosis, diabetic retinopathy, retrolental fibrioplasia, neovascular glaucoma, angiofibromas, hemangiomas, Karposi's sarcoma, and neurodegenerative disorders. Cellular proliferative disorders are described, for example, in U.S. Pat. Nos. 5,639,600, 7,087,648, and 7,217,737, hereby incorporated by reference.

By “chemotherapeutic agent” is meant an agent that may be used to destroy a cancer cell or to slow, arrest, or reverse the growth of a cancer cell. Chemotherapeutic agents include, e.g., L-asparaginase, bleomycin, busulfan carmustine (BCNU), chlorambucil, cladribine (2-CdA), CPT1 1 (irinotecan), cyclophosphamide, cytarabine (Ara-C), dacarbazine, daunorubicin, dexamethasone, doxorubicin (adriamycin), etoposide, fludarabine, 5-fluorouracil (5FU), hydroxyurea, idarubicin, ifosfamide, interferon-a (native or recombinant), levamisole, lomustine (CCNU), mechlorethamine (nitrogen mustard), melphalan, mercaptopurine, methotrexate, mitomycin, mitoxantrone, paclitaxel, pentostatin, prednisone, procarbazine, tamoxifen, taxol-related compounds, 6-thiogaunine, topotecan, vinblastine, vincristine, cisplatinum, carboplatinum, oxaliplatinum, or pemetrexed.

By “comparing” or “compared” is meant to include the act of providing, documenting, or memorializing data, information, or results relating to the same parameter from a test sample and a control sample. “Comparing” or “compared” also includes comparisons made indirectly.

By “control” or “control sample” is meant a biological sample representative or obtained from a healthy subject that has not been diagnosed with a cellular proliferative disorder. A control or control sample may have been previously established based on measurements from healthy subjects that have not been diagnosed with a cellular proliferative disorder. Further, a control sample can be defined by a specific age, sex, ethnicity, or other demographic parameters. By “control gene copy number” of PHGDH is meant the gene copy number of the PHGDH gene in a control or control sample that is typical of the general population of healthy subjects that have not been diagnosed with a cellular proliferative disorder. In some embodiments, the control is implicit in the particular measurement. For example, a typical control level for a gene (i.e., control gene copy number) is two copies per cell. An example of an implicit control is where a detection method can only detect a PHGDH gene copy number when the copy number is higher than the typical control level. Other instances of such controls are within the knowledge of the skilled artisan.

By “decrease” is meant to reduce by at least 10%, 20%, 30%, 40%, 50%, 60%, 70%, 75%, 80%, 85%, 90%, 95%, or more. A decrease can refer, for example, to the symptoms of the disorder being treated or to the levels or biological activity of a polypeptide or nucleic acid of the invention.

By “expression” is meant the detection of a nucleic acid molecule or polypeptide by standard art known methods. For example, polypeptide expression is often detected by Western blotting, DNA expression is often detected by Southern blotting or polymerase chain reaction (PCR), and RNA expression is often detected by Northern blotting, PCR, or RNase protection assays.

By “functional fragment” is meant a portion of a polypeptide or nucleic acid molecule that contains at least 5%, 10%, 20%, 30%, 40%, 50%, 60%, 70%, 80%, 90%, 95%, or more of the entire length of a nucleic acid molecule or polypeptide (e.g., PHGDH, PSAT, or PSPH) that maintains biological activity. For example, a functional fragment of the PHGDH polypeptide may contain 10, 20, 30, 40, 50, 60, 70, 80, 90, 100, 200, 300, 400, 500, or more amino acid residues, up to the full-length of the PHGDH polypeptide (NCBI Reference Sequence: NP_{—}006614.2; SEQ ID NO: 1).

By “increase” is meant to augment by at least 10%, 20%, 30%, 40%, 50%, 60%, 70%, 75%, 80%, 85%, 90%, 95%, or more. An increase can refer, for example, to the symptoms of the disorder being treated or to the levels or biological activity of a polypeptide or nucleic acid of the invention.

By “inhibitor” is meant any small molecule, nucleic acid molecule, peptide or polypeptide, or fragments thereof that reduces or inhibits the expression levels or biological activity of a protein or nucleic acid molecule by at least 10%, 20%, 30%, 40%, 50%, 60%, 70%, 80%, 90%, 95%, or more. Non-limiting examples of inhibitors include, e.g., small molecule inhibitors, antisense oligomers (e.g., morpholinos), double-stranded RNA for RNA interference (e.g., short interfering RNA (siRNA)), microRNA, aptamers, compounds that decrease the half-life of an mRNA or protein, compounds that decrease transcription or translation, dominant-negative fragments or mutant polypeptides that block the biological activity of wild-type protein, and peptidyl or non-peptidyl compounds (e.g., antibodies or antigen-binding fragments thereof) that bind to a protein.

By “pharmaceutical composition” is meant a composition containing a therapeutic agent of the invention (e.g., an inhibitor of PHGDH) formulated with a pharmaceutically acceptable excipient and manufactured for the treatment or prevention of a disorder in a subject. Pharmaceutical compositions can be formulated, for example, for oral administration in unit dosage form (e.g., a tablet, capsule, caplet, gel-cap, or syrup), for topical administration (e.g., as a cream, gel, lotion, or ointment), for intravenous administration (e.g., as a sterile solution, free of particulate emboli, and in a solvent system suitable for intravenous use), or for any other formulation described herein.

By “pharmaceutically acceptable carrier” is meant a carrier that is physiologically acceptable to the treated subject while retaining the therapeutic properties of the therapeutic agent (e.g., an inhibitor of PHGDH) with which it is administered. One exemplary pharmaceutically acceptable carrier substance is physiological saline. Other physiologically acceptable carriers and their formulations are known to one skilled in the art.

By “pharmaceutically acceptable salt” is meant salts that are suitable for use in contact with the tissues of a subject without undue toxicity, irritation, or allergic response. Pharmaceutically acceptable salts are well known in the art. The salts can be prepared in situ during the final isolation and purification of the therapeutic agents of the invention or separately by reacting the free base function with a suitable organic acid. Representative acid addition salts include, e.g., acetate, ascorbate, aspartate, benzoate, citrate, digluconate, fumarate, glucoheptonate, glycerophosphate, hemisulfate, heptonate, hexanoate, hydrobromide, hydrochloride, hydroiodide, lactate, malate, maleate, malonate, mesylate, oxalate, phosphate, succinate, sulfate, tartrate, thiocyanate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to, ammonium, tetramethylammonium, tetraethylammonium, methylamine, dimethylamine, trimethylamine, triethylamine, and ethylamine.

By “reduce or inhibit” is meant the ability to cause an overall decrease of 10%, 20%, 30%, 40%, 50%, 60%, 70%, 75%, 80%, 85%, 90%, 95%, or greater. For therapeutic applications, to “reduce or inhibit” can refer to the symptoms of the disorder being treated or the presence or extent of a disorder being treated.

By “reducing the likelihood of is meant reducing the severity, the frequency, or both the severity and frequency of a cellular proliferative disorder or symptoms thereof Reducing the likelihood of a cellular proliferative disorder is synonymous with prophylaxis or the chronic treatment of a cellular proliferative disorder.

By “reference” is meant any sample, standard, or level that is used for comparison purposes. A “normal reference sample” can be a prior sample taken from the same subject prior to the onset of a disorder (e.g., a cellular proliferation disorder), a sample from a subject not having the disorder, a subject that has been successfully treated for the disorder, or a sample of a purified reference polypeptide at a known normal concentration. By “reference standard or level” is meant a value or number derived from a reference sample. A normal reference standard or level can be a value or number derived from a normal subject that is matched to a sample of a subject by at least one of the following criteria: age, weight, disease stage, and overall health. A “positive reference” sample, standard, or value is a sample, standard, value, or number derived from a subject that is known to have a disorder (e.g., a cellular proliferation disorder) that is matched to a sample of a subject by at least one of the following criteria: age, weight, disease stage, and overall health.

By “subject” is meant any animal, e.g., a mammal (e.g., a human). A subject who is being treated for, e.g., a cellular proliferative disorder (e.g., cancer and obesity) is one who has been diagnosed by a medical practitioner as having such a condition. Diagnosis may be performed by any suitable means. A subject of the invention may be one that has not yet been diagnosed with a cellular proliferative disorder. A subject of the invention may be identified as one having an amplification of the PHGDH gene. One of skill in the art will understand that subjects treated using the compositions or methods of the present invention may have been subjected to standard tests or may have been identified without examination as one at high risk due to the presence of one or more risk factors, such as age, genetics, or family history.

By “systemic administration” is meant any non-dermal route of administration and specifically excludes topical and transdermal routes of administration.

By “therapeutic agent” is meant any agent that produces a healing, curative, stabilizing, or ameliorative effect.

By “treating” is meant administering a pharmaceutical composition for prophylactic and/or therapeutic purposes. Prophylactic treatment may be administered, for example, to a subject who is not yet ill, but who is susceptible to, or otherwise at risk of, a particular disorder, e.g., a cellular proliferation disorder (e.g., cancer and obesity). Therapeutic treatment may be administered, for example, to a subject already suffering from a disorder in order to improve or stabilize the subject's condition. In some instances, as compared with an equivalent untreated control, treatment may ameliorate a disorder or a symptom thereof by, e.g., 5%, 10%, 20%, 30%, 40%, 50%, 60%, 70%, 80%, 90%, 95%, or 100% as measured by any standard technique. In some instances, treating can result in the inhibition of a disease, the healing of an existing disease, and the amelioration of a disease.

Other features and advantages of the invention will be apparent from the following detailed description, the claims, and the drawings.

### BRIEF DESCRIPTION OF THE DRAWINGS

FIG. 1 is the polypeptide sequence of phosphoglycerate dehydrogenase (PHGDH; NCBI Reference Sequence: NP_{—}006614.2) (SEQ ID NO: 1).

FIG. 2 is the mRNA sequence of PHGDH (NCBI Reference Sequence: NM_{—}006623.3) (SEQ ID NO: 2).

FIG. 3 is the polypeptide sequence of phosphoserine aminotransferase (PSAT; NCBI Reference Sequence: NP_{—}478059) (SEQ ID NO: 3).

FIG. 4 is the mRNA sequence of PSAT (NCBI Reference Sequence: NM_{—}058179) (SEQ ID NO: 4).

FIG. 5 is the polypeptide sequence of phosphoserine phosphatase (PSPH; NCBI Reference Sequence: NP_{—}004568) (SEQ ID NO: 5).

FIG. 6 is the mRNA sequence of PSPH (NCBI Reference Sequence: NM_{—}004577.3) (SEQ ID NO: 6).

FIG. 7A is a schematic of an alternate pathway in glycolysis using phosphoenolpyruvate (PEP)-dependent regulation of phosphoglycerate mutase (PGAM). FIG. 7B is a graph of a computer simulation of an alternate glycolytic pathway. Increasing the rate of PEP-dependent PGAM phosphorylation predicts an accumulation of 3-phosphoglycerate (3-PG). FIGS. 7C, 7D, and 7E are bar graphs showing relative glucose labeling in the serine biosynthetic pathway versus PEP in H1299 cells (FIG. 7C), HEK293T cells (FIG. 7D), and MCF10a cells (FIG. 7E).

FIG. 8A is an array-based comparative genome hybridization (CGH) of chromosome 1 in the SK-Mel628 melanoma cell line. Focal amplification of PHGDH is observed at the 1p12 locus (Source: Sanger Institute Cancer Genome Project). FIG. 8B shows the effect of PHGDH RNA interference on cell growth. Rate constants for the growth of the parental cell line, PHGDH shRNA knockdown 1, and PHGDH knockdown 2 are plotted. Western blots of PHGDH protein levels confirm knockdown of the PHGDH gene. FIG. 8C is a graph showing that serine enhances cell growth in PK-M1 and PK-M2 expressing H1299 cells, demonstrating that these cells can utilize serine from their environment. FIG. 8D is a graph showing the failure of serine rescue in PHGDH knockdown (A8) cells at 5×, 50×, and 100× relative serine concentration with respect to serine concentration in RPMI. Additional serine enhances growth in control cells.

FIG. 9A is a graph showing that cells with PHGDH amplification (TT cells) are more sensitive to PHGDH knockdown than other cells that express PHGDH (H1299 cells). FIG. 9B is a Western blot using an antibody against PHGDH that shows that PHGDH expression alone does not predict which cell lines are sensitive to PHGDH knockdown. H1299 cells and MCF10a cells both express PHGDH; however, H1299 cells are less sensitive to PHGDH knockdown and MCF10a cells are insensitive to PHGDH knockdown. In contrast, Sk-Mel-28 cells, which harbor PHGDH gene amplification, show similar expression levels to non-amplified cell lines and are sensitive to PHGDH knockdown. FIG. 9C is a Western blot using a PHGDH antibody showing that both MCF10a and Sk-Mel-28 cells express PHGDH and that this expression can be reduced using two different shRNAs. FIG. 9D is a graph showing the rate constant for cell doubling for MDF10a and Sk-Mel-28 cells. The growth rate of MCF10a cells does not change when PHGDH is knocked down, whereas Sk-Mel-28 cells show a decrease in the rate of growth that is dependent on the degree of PHGDH knockdown.

FIGS. 10A and 10B are graphs showing enzyme activity of purified PHGDH in the presence of NAD⁺ as the oxidizing agent (FIG. 10A) and NADP⁺ as the oxidizing agent (FIG. 10B). FIG. 10C is a graph depicting a 5′ 3H-glucose tracing experiment. NADPH production from glucose through the PHGDH-mediated serine biosynthesis pathway is observed. FIG. 10D shows the co-injection of NADPH ³H standard. FIG. 10E is a comparison of crystal structures of phosphoglycerate dehydrogenase (left) bound to NAD⁺ and its homolog glyoxylate reductase bound to NADP⁺ (right).

FIG. 11 is the genomic DNA sequence of PHGDH (NCBI Reference Sequence: NG_{—}009188.1) (SEQ ID NO: 7).

FIG. 12A. shows the spectral bins of [¹H, ¹³C] HSQC NMR of [U-¹³C] glucose-labeled cell extracts sorted by intensity in standard units (z-score). The four highest intensity peaks correspond to metabolites lactate, alanine, and glycine respectively.

FIG. 12B shows the relative intensity of ¹³C glycine peak normalized to an internal 50 mM DSS standard in HEK293T, H1299, and MCF-10a cells.

FIG. 12C is a schematic diagram of diversion of glucose metabolism into serine and glycine metabolism at the 3-phosphoglycerate (3PG) step through PHGDH.

FIG. 12D shows the time courses (0, 5, 10, 15, 30 minutes) of U-13C labeling intensities of thirteen metabolites from [U-13C] glucose labeling experiments measured with targeted LC/MS relative to baseline level at time zero.

FIG. 12E is a comparison of 3-phosphoserine (pSER) and phosphoenolpyruvate (PEP) labeling kinetics of [U-13C] glucose relative to baseline level at time zero with targeted LC/MS.

FIG. 12F shows the relative glucose flux into serine biosynthesis measured by steady-state labeling of [U-13C] glucose into serine with targeted LC/MS. The fraction of labeled to unlabeled glucose-derived metabolites ¹³C/(¹²C+¹³C) ion intensities (glucose incorporation) is plotted for 12 metabolites. Serine is compared with respect to the glucose-labeled fraction of downstream nucleotides and other nucleotide precursors.

FIG. 12G shows the relative protein levels (as determined by Western blot analysis) of PHGDH in HEK293T, H1299, and MCF-10a cells with a Beta-actin (Actin) loading control shown below the PHGDH band. Quantitation relative to the levels in MCF-10a cells of the total intensity of the PHGDH band relative to the Actin band is shown above.

FIG. 13A is a global survey of PHGDH copy number intensity across 3131 cancers. (left) Significance of amplifications (FDR q-value) along chromosome 1p (from Telomere to Centromere) across 3131 samples is shown. Candidate oncogenes (TP73, MYCL1, and JUN) in three peak regions and corresponding FDR q-values are shown. FDR q-value of PHGDH is shown in the fourth peak region. (middle) Copy number intensity along chromosome 1p of 150 cancers containing highest intensity of PHGDH amplification that illustrates the localized intensity near the region of PHGDH is shown. Blue indicates a deleted region, white indicates a neutral region and red indicates an amplified region. (right) Magnification of a 4 MB region containing PHGDH is shown. The solid line indicates the chromosome position of the PHGDH coding region. Ratios of ion intensities (fold change) are plotted.

FIG. 13B shows the relative cell numbers of T.T. cells upon knockdown with respect to shGFP of GFP, PHGDH, PSAT, and PSPH. Error bars represent the standard deviation of n=3 independent measurements. (below) Interphase FISH analysis showing PHGDH copy number gain in T.T. cells. The green probe maps to 1p12 and includes the PHGDH coding sequence. The red probe maps to the pericentromeric region of chromosome1 (1p11.2-q11.1). (below) Relative protein levels of PHGDH, PSAT, and PSPH (as determined by Western blot analysis) in T.T. cells following expression of an shRNA against GFP (shGFP), PHGDH (shPHGDH), PSAT (shPSAT), and PSPH (shPSPH) respectively.

FIG. 13C shows PHGDH protein expression and copy number gain in three representative human tissue samples. (upper) PHGDH expression was assessed in tumor samples using Immunohistochemistry (IHC). Nuclei are shown in blue (hematoxylin) and PHGDH antibody staining is shown in brown (3-3′-Diaminobenzidine [DAB]). (lower) panels contain interphase FISH analysis that was carried out as in FIG. 2B in matched samples to assess copy number (green) relative to the pericentromeric probe (red).

FIG. 14A shows the growth assay of stable cell lines containing shGFP or shPHGDH in five human melanoma cell lines. Three (WM266-3, Malme-3M (Malme), and SkMel-28 (Sk28) contain 1p12 copy number gain and two (GAK, Carney) other melanoma cell lines are considered. (left) Western blot analysis of protein levels of PHGDH and corresponding protein levels of Actin shown as a loading control. (right) Cell numbers for shGFP and shPHGDH normalized to shGFP are plotted for each cell line. Error bars were obtained from the standard deviation of n=3 independent measurements.

FIG. 14B shows the relative amount of glucose flux into serine biosynthesis measured by steady-state labeling of [U-13C] glucose into serine with targeted LC/MS. The fraction of labeled to unlabeled glucose-derived serine to total serine, ¹³C/¹²C+¹³C, (serine incorporation) is measured in each of the five cell lines. Error bars were obtained from the standard deviation of n=3 independent measurements.

FIG. 14C shows the relative ion intensities of 3-phosphoserine (pSer) in control (shGFP) and knockdown (shPHGDH) cells normalized to intensity in knockdown shGFP cells (pSer/shGFP). Error bars were obtained from the standard deviation of n=3 independent measurements.

FIG. 14D shows the scatter plot of the ratio of intensities (fold change), versus p value (Student's t-test) of shPHGDH relative to shGFP in Sk-Mel28 cells.

FIG. 14E shows the ratio of intensities (fold change) of glycolytic intermediates upon PHGDH knockdown (shPHGDH) relative to (shGFP) in Sk-Mel28 cells. Error bars were obtained from propagation of error of the standard deviation from three independent measurements.

FIG. 15A shows the protein expression of PHGDH by Western blot analysis with Actin as a loading control for three concentrations of Doxycycline (0 μg/ml, 1 μg/ml, 2 μg/ml).

FIG. 15B shows the pSER integrated intensities in −Dox (0 μg/ml) and +Dox (1 μg/ml).

FIG. 15C provides confocal images of DAPI (Blue), Laminin 5 (Green). Representative images from four acini from MCF-10A cells expressing doxycyline-inducible PHGDH without doxycycline (−Dox) or 1 μg/ml doxycyline (+Dox).

FIG. 15D shows the enhanced proliferation in the interior of PHGDH-expressing acini. Representative images from acini from MCF-10A cells expressing doxycyline-inducible PHGDH without doxycycline (No Dox) or 1 μg/ml doxycyline (1 μg/ml Dox). Confocal images of MCF-10A cells under the same conditions as in 4C with DAPI (Blue) and the proliferation marker Ki67 (Red).

FIG. 15E shows the quantification of acinar filling for 0 μg/ml, 1 μg/ml, and 2 μg/ml Dox. Each acini was scored as filled, mostly filled, mostly clear, and clear. These data are representative of multiple independent measurements.

FIG. 15F shows the loss of apical polarity in PHGDH-expressing cells. Confocal images of MCF-10A cells under the same conditions as in 4C with DAPI (Blue) and Golgi Apparatus (Green) are shown. Solid, white arrows indicate cells displaying oriented golgi apparatus. Dashed, yellow arrows indicate cells exhibiting loss of polarity. Acini with ectopic expression of wild type, but not mutant V490M, PHGDH commonly display mislocalized golgi apparatus, indicative of a lack of cell polarity.

### DETAILED DESCRIPTION OF THE INVENTION

The observation that cancer cells exhibit a major metabolic flux from glucose to serine has not previously been appreciated. We now show that inhibiting the serine biosynthetic pathway (in particular, inhibiting the expression of phosphoglycerate dehydrogenase (PHGDH)) inhibits the production of NADPH. We have discovered that PHGDH expression is required for cell growth and that cells lacking adequate PHGDH cannot be rescued by the presence of serine, supporting the hypothesis that NADPH production by PHGDH is critical for cell growth. Finally, we have determined that PHGDH is a major source of NADPH in cells.

Most tumors and cancer cell lines metabolize large amounts of glucose through a fermentative metabolism characterized by lactate production even in the presence of oxygen (aerobic glycolysis) (Warburg et al., Biochemische Zeitschrifi 152, 319-344 (1924)). Aerobic glycolysis may allow cancer cells to adapt metabolism to satisfy specific biosynthetic requirements (Vander Heiden et al., Science 324, 1029-33 (2009); Deberardinis et al., Cell Metab 7, 11-20 (2008)). This hypothesis is buttressed by evidence indicating that the final step in glycolysis catalyzed by pyruvate kinase is inhibited in cancer cells (Christofk et al., Nature 452, 181-6 (2008); Christofk et al., Nature 452, 230-3 (2008)). The selection for lower pyruvate kinase activity may allow glycolytic intermediates upstream of pyruvate kinase to be diverted into other metabolic pathways in cancer cells. Metabolomics, in conjunction with stable isotope labeling of glucose, allow for study of the pathways originating from glucose metabolism and insight as to whether utilization of specific alternative pathways is necessary for cancer cell proliferation and whether differences in individual fluxes contribute to the development of cancers.

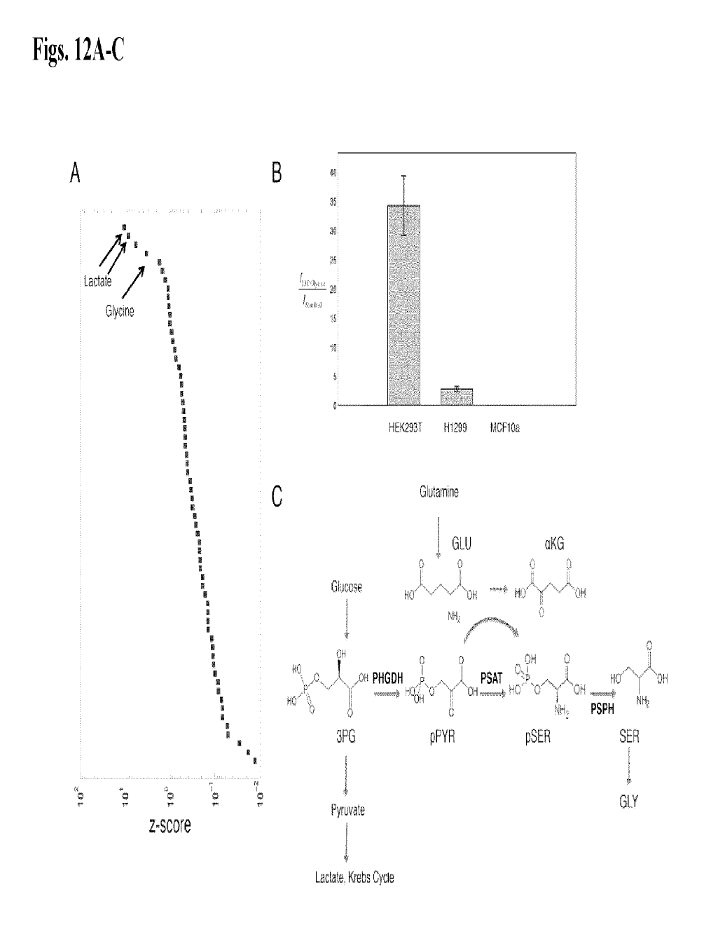

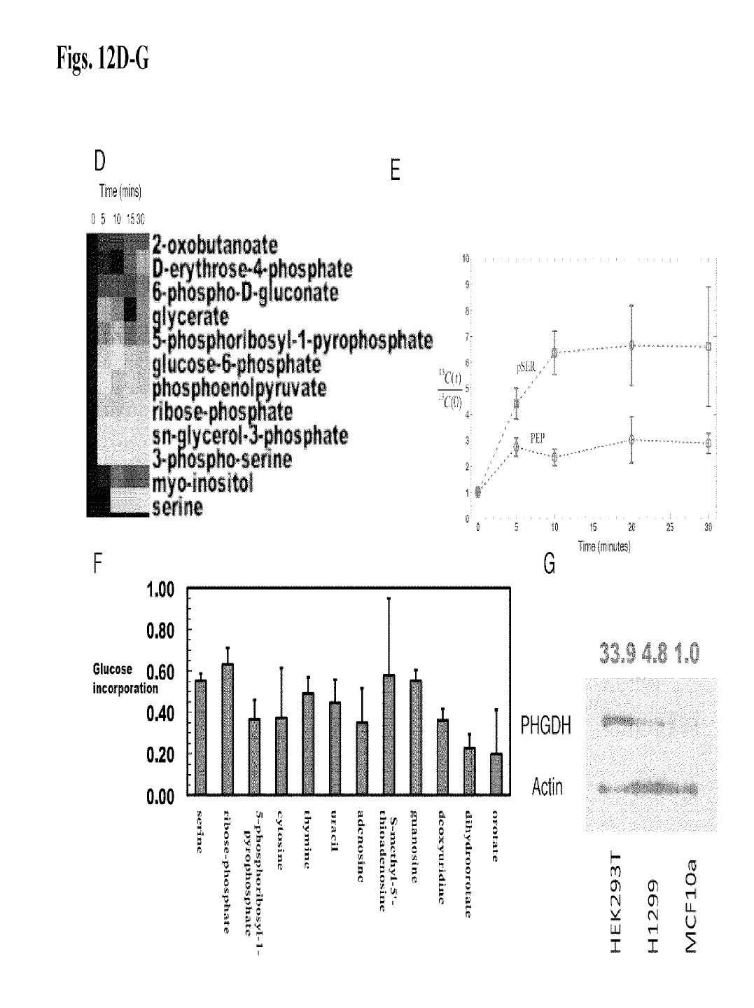

Glycine can be generated from glucose via diversion of the glycolytic intermediate, 3-phosphoglycerate (3PG), into the serine synthesis pathway and by the ultimate conversion of serine to glycine (FIG. 12C) (De Koning et al., Biochemical Journal 371, 653-661 (2003)). The first committed step in this pathway is the oxidation of 3PG to 3-phosphohydroxypyruvate (pPYR) by the enzyme phosphoglycerate dehydrogenase (PHGDH) (Achouri et al., Biochemical Journal 323, 365-370 (1997)). pPYR is transaminated by phosphoserine aminotransferase (PSAT) with glutamate as a nitrogen donor to form phosphoserine (pSER) and alpha-ketoglutarate (aKG), and pSER is then dephosphorylated by phosphoserine phosphatase (PSPH) to form serine (FIG. 12C). Serine (SER) can be directly converted to glycine (GLY) by donation of a carbon into the folate pool. This pathway defines a branching point for 3PG from glycolysis, initialized by the enzymatic activity of PHGDH, that could otherwise be metabolized to pyruvate, alanine, and lactate. Serine and glycine are intermediates in pathways for the synthesis of other amino acids, as well as lipids and nucleic acids. Flux into this pathway has been observed in cancer cells but its cancer context, stoichiometry, requirement for cell growth, and potential to promote cell transformation were unknown (Bismut et al., Biochemical Journal 308, 761-767 (1995); Snell et al., Biochemical Journal 245, 609-612 (1987); and Kit, Cancer Research 15, 715-718 (1955)). The data provided herein show that PHGDH, a focus of recurrent genomic amplification, diverts glycolysis into a specific biosynthetic pathway and that this change in metabolism can be selected for in the development of human cancer.

The diversion of glycolytic flux into de novo serine biosynthesis has a multitude of biological consequences. Metabolic pathways downstream of serine metabolism contribute to growth-promoting biosynthesis and metabolic signaling functions from the folate pool, amino acid, and lipid intermediates, and redox regulation (Schafer et al., Nature 461, 109-U118 (2009); Teperino et al., Cell Metabolism 12, 321-327; Nomura et al., Cell 140, 49-61; and Hara et al., Journal of Biological Chemistry 273, 14484-14494 (1998)). In addition, the process of diverting fluxes from 3PG out of glycolysis confers several advantages for cell growth. These include limiting ATP production, direct alterations in cellular redox status from the oxidation of 3PG, and the generation of aKG from glutamate, all of which are reported to benefit cell growth through multiple mechanisms (Vander Heiden et al., Science 329, 1492-1499 (2010); Locasale et al., Bmc Biology 8, 3; and Eng et al., Science Signaling 3, 9).

The observation that a genetic lesion can function to directly alter metabolic flux out of glycolysis provides multiple avenues for further inquiry and demonstrates that alterations in metabolism beyond increased lactate production are important events in the development of cancer.

### Cellular Proliferative Disorders

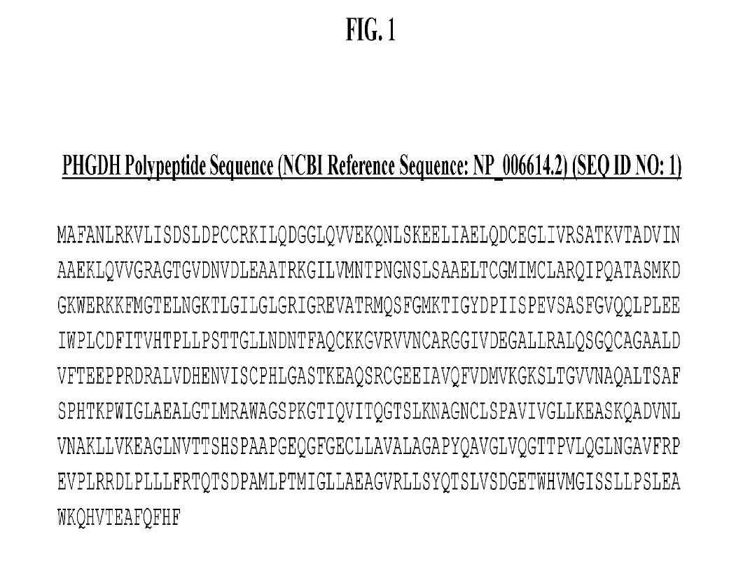

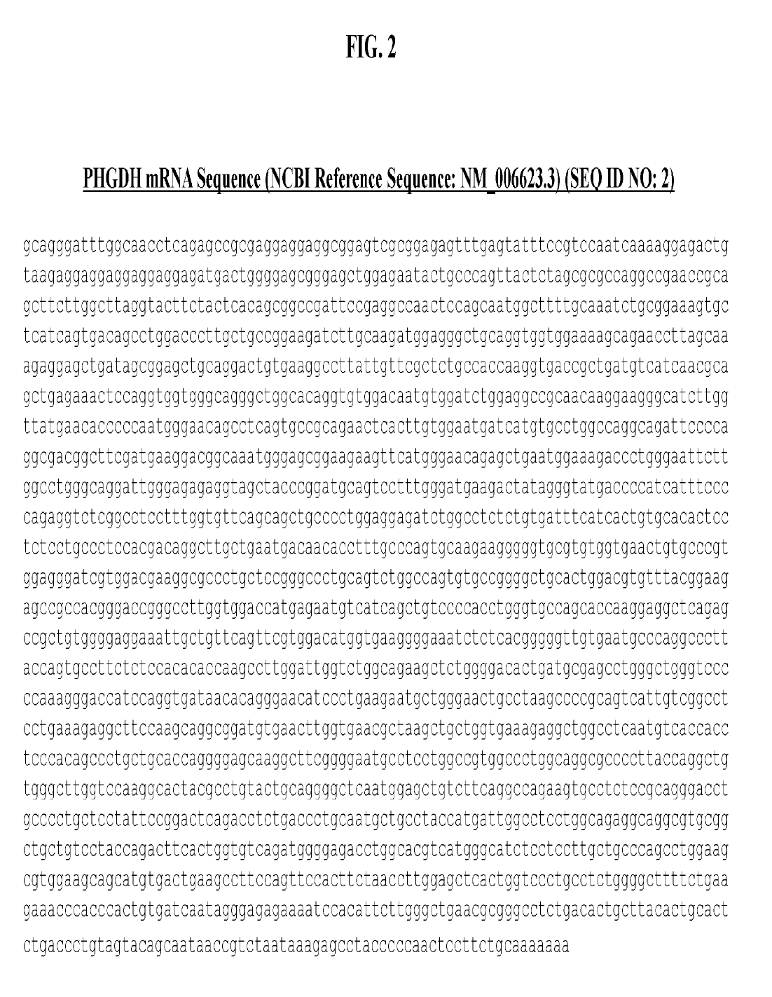

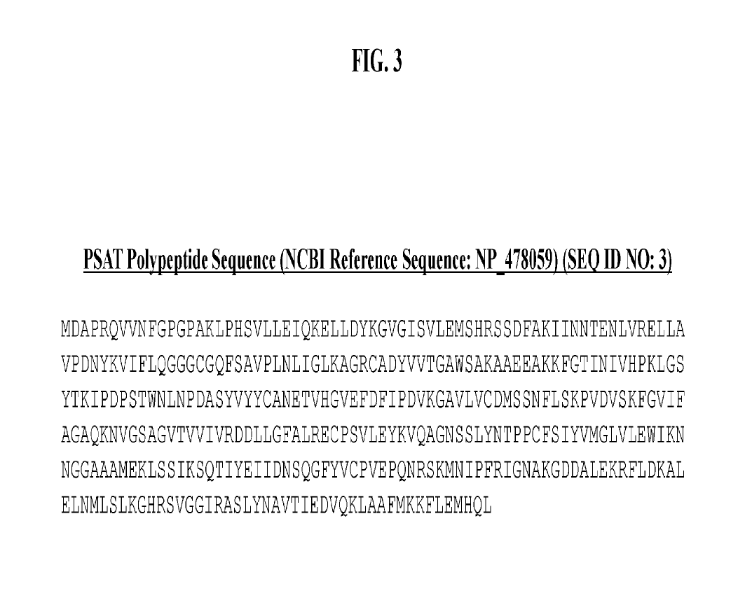

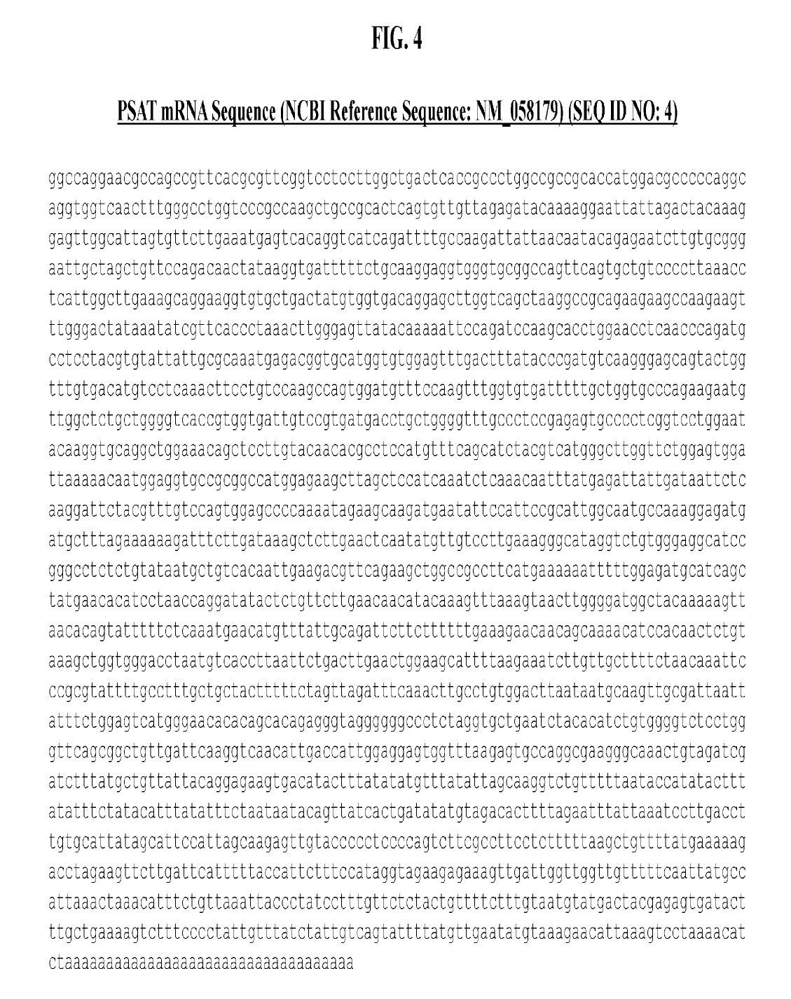

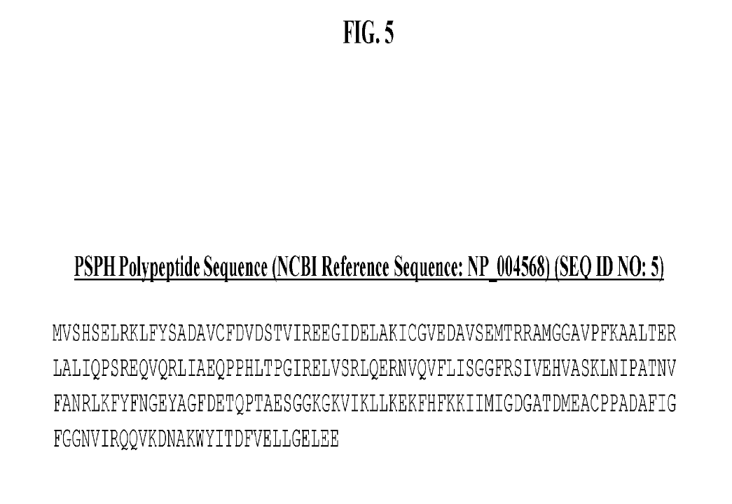

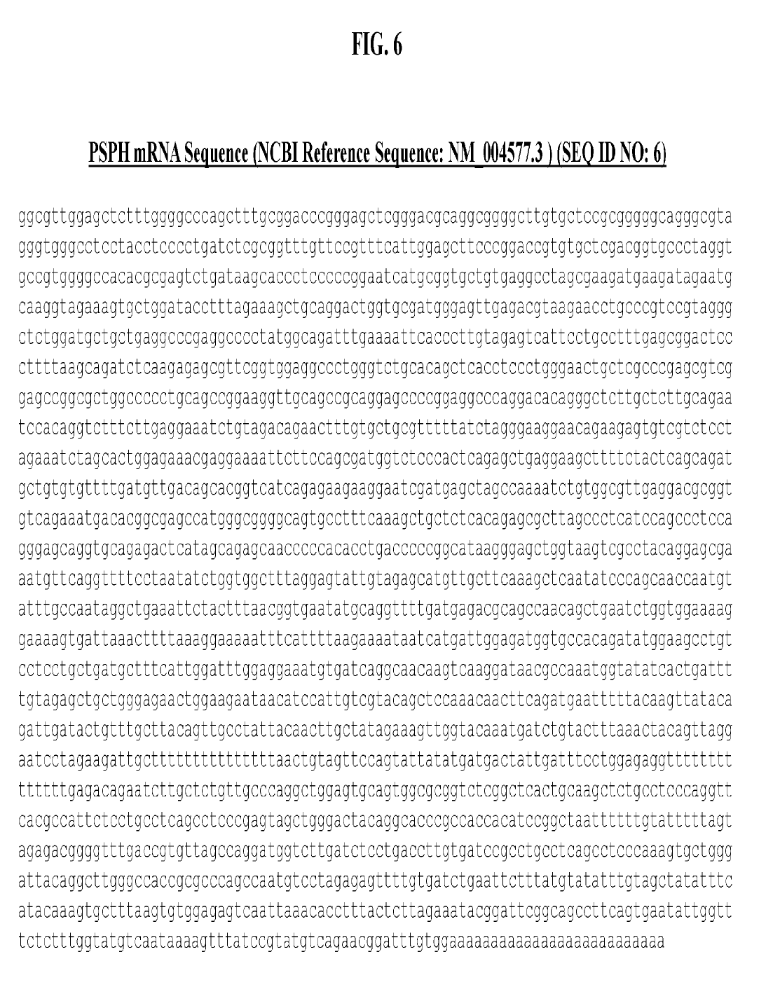

The present invention features methods and compositions for the diagnosis and prognosis of cellular proliferative disorders (e.g., cancer) and the treatment of these disorders by targeting PHGDH (FIGS. 1, 2, and 10; SEQ ID NOs: 1, 2, and 7) and other enzymes of the serine biosynthetic pathway (e.g., phosphoserine aminotransferase (PSAT; FIGS. 3 and 4; SEQ ID NOs: 3 and 4) or phosphoserine phosphatase (PSPH; FIGS. 5 and 6; SEQ ID NOs: 5 and 6)). Cellular proliferative disorders described herein include, e.g., cancer, obesity, and proliferation-dependent diseases. Such disorders may be diagnosed using methods known in the art.

Cancer

Cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma).

Other Proliferative Diseases

Other proliferative diseases include, e.g., obesity, benign prostatic hyperplasia, psoriasis, abnormal keratinization, lymphoproliferative disorders (e.g., a disorder in which there is abnormal proliferation of cells of the lymphatic system), chronic rheumatoid arthritis, arteriosclerosis, restenosis, and diabetic retinopathy. Proliferative diseases are described in U.S. Pat. Nos. 5,639,600 and 7,087,648, hereby incorporated by reference.

Diagnostics

The present invention features methods and compositions to diagnose a cellular proliferative disorder and monitor the progression of such a disorder. For example, the methods can include determining PHGDH gene copy number in a biological sample and comparing the gene copy number to a normal reference.

Determination of the genomic copy number of PHGDH has many advantages over determining, for example, the protein level or mRNA expression level of PHGDH in a cell. Many cells, including non-cancer cells, express PHGDH. However, expression at the protein or mRNA level alone may not be sufficient to identify those cancers which were selected specifically to have a genetic event leading to increased PHGDH expression. In contrast, amplification of the gene suggests a genetic selection for those cells which are dependent on higher copy number of PHGDH for growth. In these cells, PHGDH expression provides a growth advantage that enables the clonal expansion of cells with the genomic alteration leading to increased expression. Thus, examination of the genomic copy number can identify those cancers which will respond to therapy targeting PHGDH.

The presence of a gene that has undergone amplification in a biological sample is evaluated by determining the copy number of the genes, e.g., the number of DNA sequences in a cell encoding the target protein. Generally, a normal diploid cell has two copies of a given autosomal gene. The copy number can be increased, however, by gene amplification or duplication, for example, in cancer cells, or reduced by deletion. Methods of evaluating the copy number of a particular gene are well known in the art and include, without limitation, hybridization- and amplification-based assays.

Any of a number of hybridization-based assays can be used to detect the copy number of, for example, a PHGDH gene in a biological sample. One such method is Southern blotting, where the genomic DNA may be fragmented, separated electrophoretically, transferred to a membrane, and subsequently hybridized to a PHGDH-specific probe. Comparison of the intensity of the hybridization signal from the probe for the target region with a signal from a control probe from a region of normal non-amplified, single-copied genomic DNA in the same genome provides an estimate of the relative PHGDH gene copy number, corresponding to the specific probe used. An increased signal compared to a control represents the presence of amplification.

Another methodology for determining the copy number of the PHGDH gene in a sample is in situ hybridization, for example, fluorescence in situ hybridization (FISH) (see, e.g., Angerer et al., Methods Enzymol. 152:649-661, 1987). Generally, in situ hybridization includes the following steps: (1) fixation of a biological sample to be analyzed; (2) pre-hybridization treatment of the biological sample to increase accessibility of target DNA and to reduce non-specific binding; (3) hybridization of the mixture of nucleic acids to the nucleic acid in the biological sample; (4) post-hybridization washes to remove nucleic acid fragments not bound in the hybridization; and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

Another methodology for determining the number of gene copies is comparative genomic hybridization (CGH). In comparative genomic hybridization methods, a “test” collection of nucleic acids is labeled with a first label, while a second collection (for example, from a normal cell or tissue) is labeled with a second label. The ratio of hybridization of the nucleic acids is determined by the ratio of the first and second labels binding to each fiber in an array. Differences in the ratio of the signals from the two labels, for example, due to gene amplification in the test collection are detected, and the ratio provides a measure of, for example, the gene copy number corresponding to the specific probe used. A cytogenetic representation of DNA copy-number variation can be generated by CGH, which provides fluorescence ratios along the length of chromosomes from differentially labeled test and reference genomic DNAs.

Hybridization protocols suitable for use with the methods of the invention are described, for example, in Albertson, EMBO J. 3:1227-1234, 1984, and Pinkel et al., Proc. Nail. Acad. Sci. USA 85:9138-9142, 1988, hereby incorporated by reference.

Amplification-based assays also can be used to measure the copy number of the PHGDH gene. In such assays, the corresponding PHGDH nucleic acid sequences act as a template in an amplification reaction (for example, a polymerase chain reaction or PCR). In a quantitative amplification, the amount of amplification product will be proportional to the amount of template in the original sample. Comparison to appropriate controls provides a measure of the copy number of the PHGDH gene, corresponding to the specific probe used, according to the principles discussed above. Methods of real-time quantitative PCR using TaqMan probes are well known in the art. Detailed protocols for real-time quantitative PCR are provided, for example, in Gibson et al., Genome Res. 6:995-1001, 1996, and in Heid et al., Genome Res. 6:986-994, 1996.

A TaqMan-based assay also can be used to quantify PHGDH polynucleotides. TaqMan-based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification.

Other suitable amplification methods include, but are not limited to, ligase chain reaction (LCR) (see, e.g., Wu and Wallace, Genomics 4:560-569, 1989; Landegren et al., Science 241: 1077-1080, 1988; and Barringer et al., Gene 89:117-122, 1990), transcription amplification (see, e.g., Kwoh et al., Proc. Natl. Acad. Sci. USA 86:1173-1177, 1989), self-sustained sequence replication (see, e.g., Guatelli et al., Proc. Natl. Acad. Sci. USA 87:1874-1878, 1990), dot PCR, and linker adapter PCR.

DNA copy number may also be determined using microarray-based platforms (e.g., single-nucleotide polymorphism (SNP) arrays), as microarray technology offers high resolution. For example, traditional CGH generally has a 20 Mb-limited mapping resolution, whereas, in microarray-based CGH, the fluorescence ratios of the differentially labeled test and reference genomic DNAs provide a locus-by-locus measure of DNA copy-number variation, thereby achieving increased mapping resolution. Details of various microarray methods can be found in the literature. See, for example, U.S. Pat. No. 6,232,068 and Pollack et al., Nat. Genet. 23:41-46, 1999.

Detection of amplification, overexpression, or overproduction of, for example, a PHGDH gene or gene product can also be used to provide prognostic information or guide therapeutic treatment. Such prognostic or predictive assays can be used to determine prophylactic treatment of a subject prior to the onset of symptoms of, e.g., a cellular proliferative disorder.

The methods of the present invention can also include the detection and measurement of, for example, PHGDH (or a functional fragment thereof) expression or biological activity.

For diagnoses based on relative levels of PHGDH, a subject with a disorder (e.g., a cellular proliferative disorder) will show an alteration (e.g., an increase of 10%, 20%, 30%, 40%, 50%, 60%, 70%, 80%, 90%, or more) in the amount of the PHGDH expressed or an alteration (e.g., an increase of 10%, 20%, 30%, 40%, 50%, 60%, 70%, 80%, 90%, or more) in PHGDH biological activity compared to a normal reference. A normal reference sample can be, for example, a prior sample taken from the same subject prior to the development of the disorder or of symptoms suggestive of the disorder, a sample from a subject not having the disorder, a sample from a subject not having symptoms of the disorder, or a sample of a purified reference polypeptide at a known normal concentration (i.e., not indicative of the disorder).

Standard methods may be used to measure levels of PHGDH in a biological sample, including, but not limited to, urine, blood, serum, plasma, saliva, amniotic fluid, or cerebrospinal fluid. Such methods include immunoassay, ELISA, Western blotting, and quantitative enzyme immunoassay techniques, such as IHC.

The diagnostic methods described herein can be used individually or in combination with any other diagnostic method described herein for a more accurate diagnosis of the presence or severity of a disorder (e.g., a cellular proliferation disorder). Examples of additional methods for diagnosing such disorders include, e.g., examining a subject's health history, immunohistochemical staining of tissues, computed tomography (CT) scans, or culture growths.

### Screening Assays

As discussed above, we have discovered that inhibiting enzymes of the serine biosynthetic pathway (e.g., PHGDH, PSAT, and PSPH) inhibits the production of NADPH and inhibits cells proliferation. Based on these discoveries, such enzymes or functional fragments thereof and the nucleic acids that encode these enzymes or functional fragments thereof are useful targets for high-throughput, low-cost screening of candidate compounds to identify those that modulate, alter, or decrease (e.g., by at least 10%, 20%, 30%, 40%, 50%, 60%, 70%, 80%, 90%, 95%, or more) the expression or biological activity of these enzymes. Compounds that decrease the expression or biological activity of, for example, PHGDH can be used for the treatment of a cellular proliferative disorder. Candidate compounds can be tested for their effect on PHGDH using assays known in the art or described in the Examples below.

For example, we have discovered that inhibition of PHGDH inhibits the production of NADPH. Accordingly, to identify inhibitors of PHGDH, conversion of NADP⁺ to NADPH can be monitored (e.g., in vitro or in vivo) when PHGDH is contacted with a candidate compound. A decrease in the conversion of NADP to NADPH may indicate, for example, that the candidate compound is an inhibitor of PHGDH. The conversion of NADP⁺ to NADPH can be monitored directly or indirectly, for example, using diaphorase as a detection enzyme system or any other methods known in the art. The conversion of NADP to NADPH can also monitored through monitoring the consumption of NADP⁺ or the production of NADPH. The consumption of NADP⁺ or the production of NADPH can be monitored directly or indirectly.

In general, candidate compounds are identified from large libraries of natural product or synthetic (or semi-synthetic) extracts, chemical libraries, or from polypeptide or nucleic acid libraries, according to methods known in the art. Those skilled in the field of drug discovery and development will understand that the precise source of test extracts or compounds is not critical to the screening procedure(s) of the invention.

### Therapeutic Agents

Therapeutic agents useful in the methods of the invention include any compound that can reduce or inhibit the biological activity or expression level of a phosphoglycerate dehydrogenase (PHGDH) polypeptide or PHGDH nucleic acid molecule. PHGDH activity is influenced by the product of the enzyme, phosphohydroxypruvate. Phosphohydroxypyruvate is metabolized to serine by two enzymes, phosphoserine aminotransferase (PSAT) and phosphoserine phosphatase (PSPH). Thus, targeting these enzymes in the serine biosynthetic pathway would inhibit NADPH production by PHGDH.

Exemplary inhibitor compounds include, but are not limited to, small molecule inhibitors, antisense nucleobase oligomers (e.g., morpholinos), double-stranded RNA for RNA interference (e.g., short interfering RNA (siRNA)), microRNA, aptamers, compounds that decrease the half-life of an mRNA or protein, compounds that decrease transcription or translation, dominant-negative fragments or mutant polypeptides that block the biological activity of wild-type protein, and peptidyl or non-peptidyl compounds (e.g., antibodies or antigen-binding fragments thereof) that bind to a protein (e.g., PHGDH).

Desirably, inhibitor compounds will reduce or inhibit the biological activity or expression levels of polypeptide or nucleic acid (e.g., a PHGDH polypeptide or nucleic acid) by at least 10%, 25%, 30%, 40%, 50%, 60%, 70%, 80%, 90%, 95%, 96%, 97%, 98%, 99%, or more. The inhibitor compound may reduce or inhibit cell proliferation, the reduction of NADP⁺ to NAPDH, and the catalysis of 3-phosphoglycerate to 3-phosphohydroxypyruvate by at least 10%, 20%, 25%, 30%, 40%, 50%, 60%, 70%, 80%, 90%, 95%, 96%, 97%, 98%, 99%, or more.

Nucleic Acid Molecules

The therapeutic agent of the invention (e.g., an inhibitor of PHGDH) may be a nucleic acid molecule. Such inhibitory nucleic acid molecules are capable of mediating the downregulation of the expression of a polypeptide or nucleic acid encoding the same (e.g., a PHGDH polypeptide or nucleic acid) or mediating a decrease in the activity of a polypeptide of the invention. Examples of the inhibitory nucleic acids of the invention include, without limitation, antisense oligomers (e.g., morpholinos), dsRNAs (e.g., siRNAs and shRNAs), microRNAs, and aptamers.

Antisense Oligomers

The present invention features antisense oligomers to any of the polypeptides of the invention (e.g., PHGDH, PSAT, or PSPH) and the use of such oligomers to downregulate expression of mRNA encoding the polypeptide. By binding to the complementary nucleic acid sequence (i.e., the sense or coding strand), antisense oligomers are able to inhibit protein expression, presumably through the enzymatic cleavage of the RNA strand by RNase H. Desirably, the antisense oligomer is capable of reducing polypeptide expression in a cell by at least 10%, 20%, 30%, 40%, 50%, 60%, 70%, 80%, 90%, or greater, relative to cells treated with a control oligonucleotide. Methods for selecting and preparing antisense oligomers are well known in the art. Methods for assaying levels of protein expression are also well known in the art and include, for example, Western blotting, immunoprecipitation, and ELISA.

One example of an antisense oligomer is a morpholino oligomer. Morpholinos act by “steric blocking” or binding to a target sequence within an RNA and blocking molecules, which might otherwise interact with the RNA.

Morpholinos are synthetic molecules that bind to complementary sequences of RNA by standard nucleic acid base-pairing. While morpholinos have standard nucleic acid bases, those bases are bound to morpholine rings instead of deoxyribose rings and linked through phosphorodiamidate groups instead of phosphates. Because of their unnatural backbones, morpholinos are not recognized by cellular proteins. Nucleases do not degrade morpholinos, and morpholinos do not activate innate immune responses. Morpholinos are also not known to modify methylation of DNA. Accordingly, morpholinos that are directed to any part of a polypeptide of the invention (e.g., PHGDH, PSAT, or PSPH) and that reduce or inhibit the expression levels or biological activity of the polypeptide are particularly useful in the methods and compositions of the invention.

dsRNAs

The present invention also features the use of double stranded RNAs including, but not limited to, siRNAs and shRNAs. Short, double-stranded RNAs may be used to perform RNA interference (RNAi) to inhibit the expression of a polypeptide of the invention (e.g., PHGDH, PSAT, or PSPH). RNAi is a form of post-transcriptional gene silencing initiated by the introduction of double-stranded RNA (dsRNA). Short 15 to 32 nucleotide double-stranded RNAs, known generally as “siRNAs,” “small RNAs,” or “microRNAs” are effective at down-regulating gene expression in nematodes (Zamore et al., Cell 101: 25-33) and in mammalian tissue culture cell lines (Elbashir et al., Nature 411:494-498, 2001). The further therapeutic effectiveness of this approach in mammals was demonstrated in vivo by McCaffrey et al. (Nature 418: 38-39, 2002). The small RNAs are at least 15 nucleotides, preferably 16, 17, 18, 19, 20, 21, 22, 23, 24, 25, 26, 27, 28, 29, 30, 31, 32, 33, 34, 35 nucleotides in length and even up to 50 or 100 nucleotides in length (inclusive of all integers in between). Such small RNAs that are substantially identical to or complementary to any region of a polypeptide described herein are included in the invention. Non-limiting examples of small RNAs are substantially identical to (e.g., 60%, 65%, 70%, 75%, 80%, 85%, 90%, 95%, 96%, 97%, 98%, 99%, or 100% sequence identity) or complementary to the PHGDH (SEQ ID NO: 2), PSAT (SEQ ID NO: 4), or PSPH (SEQ ID NO: 6) nucleic acid sequence. It should be noted that longer dsRNA fragments that are processed into small RNAs may be used. Small RNAs to be used as inhibitors of the invention can be identified by their ability to decrease polypeptide expression levels or biological activity performing assays known in the art or provided herein. Small RNAs can also include short hairpin RNAs in which both strands of a siRNA duplex are included within a single RNA molecule.

The specific requirements and modifications of small RNAs are known in the art and are described, for example, in PCT Publication No. WO 01/75164, and U.S. Patent Application Publication Nos. 2006/0134787, 2005/0153918, 2005/0058982, 2005/0037988, and 2004/0203145, the relevant portions of which are herein incorporated by reference.

siRNA molecules can be obtained and purified through a variety of protocols known to one of skill in the art, including chemical synthesis or recombinant production using a Drosophila in vitro system. They are commercially available from companies such as Dharmacon Research Inc. or Xeragon Inc., or they can be synthesized using commercially available kits such as the Silencer™ siRNA Construction Kit from Ambion (Catalog Number 1620) or HiScribe™ RNAi Transcription Kit from New England BioLabs (Catalog Number E2000S). Alternatively, siRNA can be prepared using standard procedures for in vitro transcription of RNA and dsRNA annealing procedures.

Short hairpin RNAs (shRNAs) can also be used in the methods of the invention. shRNAs are designed such that both the sense and antisense strands are included within a single RNA molecule and connected by a loop of nucleotides. shRNAs can be synthesized and purified using standard in vitro T7 transcription synthesis. shRNAs can also be subcloned into an expression vector, which can then be transfected into cells and used for in vivo expression of the shRNA.

A variety of methods are available for transfection of dsRNA into mammalian cells. For example, there are several commercially available transfection reagents useful for lipid-based transfection of siRNAs including, but not limited to, TransIT-TKO™ (Minis, Catalog Number MIR 2150), Transmessenger™ (Qiagen, Catalog Number 301525), Oligofectamine™ and Lipofectamine™ (Invitrogen, Catalog Number MIR 12252-011 and Catalog Number 13778-075), siPORT™ (Ambion, Catalog Number 1631), DharmaFECT™ (Fisher Scientific, Catalog Number T-2001-01). Agents are also commercially available for electroporation-based methods for transfection of siRNA, such as siPORTer™ (Ambion Inc., Catalog Number 1629). Microinjection techniques may also be used. The small RNA can also be transcribed from an expression construct introduced into the cells, where the expression construct includes a coding sequence for transcribing the small RNA operably linked to one or more transcriptional regulatory sequences. Where desired, plasmids, vectors, or viral vectors can also be used for the delivery of dsRNA or siRNA, and such vectors are known in the art. Protocols for each transfection reagent are available from the manufacturer. Additional methods are known in the art and are described, for example, in U.S. Patent Application Publication No. 2006/0058255.

Aptamers

The present invention also features aptamers to the polypeptides of the invention (e.g., PHGDH) and the use of such aptamers to downregulate expression of the polypeptide or nucleic acid encoding the polypeptide. Aptamers are nucleic acid molecules that form tertiary structures that specifically bind to a target molecule. The generation and therapeutic use of aptamers are well established in the art. See, e.g., U.S. Pat. No. 5,475,096 and U.S. Patent Application Publication No. 2006/0148748. For example, a PHGDH aptamer may be a pegylated, modified oligonucleotide, which adopts a three-dimensional conformation that enables it to bind to PHGDH and inhibit the biological activity of PHGDH.

Small Molecule Therapeutic Agents

Small molecule therapeutic agents for use in the present invention can be identified using standard screening methods specific to the target (e.g., PHGDH, PSAT, or PSPH). These screening methods can also be used to confirm the activities of derivatives of compounds found to have a desired activity, which are designed according to standard medicinal chemistry approaches. After a small molecule therapeutic agent is confirmed as being active with respect to a particular target, the therapeutic agent can be tested in vitro, as well as in appropriate animal model systems.

The small molecule therapeutic agents of the present invention may be derivatives, analogs, or mimetics of substrates present in the serine biosynthetic pathway (e.g., 3-phosphoglycerate, 3-phosphohydroxypynivate, or O-phosphoserine). Examples of such compounds include, for example, 3-bromopyruvate, L-serine, and analogs or derivatives thereof.

### Therapeutic Formulations

The invention includes the use of therapeutic agents (e.g., inhibitor compounds) to treat or reduce the likelihood of developing a cellular proliferative disorder (e.g., cancer and obesity) in a subject. Thus, the present invention includes pharmaceutical compositions that include an inhibitor of PHGDH and a phannaceutically acceptable carrier, wherein said inhibitor of PHGDH is present in an amount that, when administered to a subject, is sufficient to treat or reduce the likelihood of developing a cellular proliferative disorder in said subject. In one aspect, the cellular proliferative disorder is cancer. The therapeutic agent can be administered at any time. For example, for therapeutic applications, the agent can be administered after diagnosis or detection of a cellular proliferative disorder or after the onset of symptoms of a cellular proliferative disorder. The therapeutic agent can also be administered before diagnosis or onset of symptoms of a cellular proliferative disorder in subjects that have not yet been diagnosed with a cellular proliferative disorder, but that are at risk of developing such a disorder, or after a risk of developing a cellular proliferative disorder is determined. A therapeutic agent of the invention may be formulated with a pharmaceutically acceptable diluent, carrier, or excipient in unit dosage form. Conventional pharmaceutical practice may be employed to provide suitable formulations or compositions to administer the therapeutic agent of the invention to a subject suffering from or at risk of developing a cellular proliferative disorder. Administration may begin before the patient is symptomatic. The therapeutic agent of the present invention can be formulated and administered in a variety of ways, e.g., those routes known for specific indications, including, but not limited to, topically, orally, subcutaneously, intravenously, intracerebrally, intranasally, transdermally, intraperitoneally, intramuscularly, intrapulmonary, rectally, intra-arterially, intralesionally, parenterally, or intra-ocularly. The therapeutic agent can be in the form of a pill, tablet, capsule, liquid, or sustained release tablet for oral administration; or a liquid for intravenous administration, subcutaneous administration, or injection; for intranasal formulations, in the form of powders, nasal drops, or aerosols; or a polymer or other sustained-release vehicle for local administration.

The invention also includes the use of therapeutic agent (e.g., an inhibitor of PHGDH) to treat or reduce the likelihood of developing a cellular proliferative disorder in a biological sample derived from a subject (e.g., treatment of a biological sample ex vivo) using any means of administration and formulation described herein). The biological sample to be treated ex vivo may include any biological fluid (e.g., blood, serum, plasma, or cerebrospinal fluid), cell (e.g., an endothelial cell), or tissue from a subject that has a cellular proliferative disorder or the propensity to develop a cellular proliferative disorder. The biological sample treated ex vivo with the therapeutic agent may be reintroduced back into the original subject or into a different subject. The ex vivo treatment of a biological sample with a therapeutic agent, as described herein, may be repeated in an individual subject (e.g., at least once, twice, three times, four times, or at least ten times). Additionally, ex vivo treatment of a biological sample derived from a subject with a therapeutic agent, as described herein, may be repeated at regular intervals (non-limiting examples include daily, weekly, monthly, twice a month, three times a month, four times a month, bi-monthly, once a year, twice a year, three times a year, four times a year, five times a year, six times a year, seven times a year, eight times a year, nine times a year, ten times a year, eleven times a year, and twelve times a year).

Therapeutic formulations are prepared using standard methods known in the art by mixing the active ingredient having the desired degree of purity with optional physiologically acceptable carriers, excipients or stabilizers (Remington's Pharmaceutical Sciences (20th edition), ed. A. Gennaro, 2000, Lippincott, Williams & Wilkins, Philadelphia, Pa.) in the form of lyophilized formulations or aqueous solutions. Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, the formulation contains a pharmaceutically acceptable salt (e.g., sodium chloride) at about physiological concentrations. The formulation may also contain the therapeutic agent (e.g., inhibitor of PHGDH) in the form of a calcium salt. The formulations of the invention may contain a pharmaceutically acceptable preservative. In some embodiments, the preservative concentration ranges from 0.1 to 2.0%, typically v/v. Suitable preservatives include those known in the pharmaceutical arts, including benzyl alcohol, phenol, m-cresol, methylparaben, and propylparaben. The formulations of the invention may also include a pharmaceutically acceptable surfactant, such as non-ionic detergents.

For parenteral administration, the therapeutic compound is formulated in a unit dosage injectable form (e.g., solution, suspension, emulsion) in association with a pharmaceutically acceptable parenteral vehicle. Such vehicles are inherently non-toxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives.

The dosage required depends on the choice of the route of administration; the nature of the formulation; the nature of the subject's illness; the subject's size, weight, surface area, age, and sex; other drugs being administered; and the judgment of the attending physician. For example, oral administration would be expected to require higher dosages than administration by intravenous injection. Variations in these dosage levels can be adjusted using standard empirical routines for optimization, as is well understood in the art. Administrations can be single or multiple (e.g., 2, 3, 6, 8, 10, 20, 50, 100, 150, or more). Encapsulation of the therapeutic compound in a suitable delivery vehicle (e.g., polymeric microparticles or implantable devices) may increase the efficiency of delivery, particularly for oral delivery.

As described above, the dosage of the therapeutic agent will depend on other clinical factors such as weight and condition of the subject and the route of administration of the compound. For treating subjects, between approximately 0.001 mg/kg to 500 mg/kg body weight of the therapeutic agent (e.g., inhibitor of PHGDH) can be administered. A more preferable range is 0.01 mg/kg to 50 mg/kg body weight with the most preferable range being from 1 mg/kg to 25 mg/kg body weight. Depending upon the half-life of the therapeutic agent in the particular subject, the compound can be administered between several times per day to once a week. The methods of the present invention provide for single as well as multiple administrations, given either simultaneously or over an extended period of time.

Alternatively, a polynucleotide containing a nucleic acid sequence which is itself or encodes a therapeutic agent (e.g., an inhibitory nucleic acid molecule that inhibits the expression of a nucleic acid molecule encoding a polypeptide of the invention (e.g., PHGDH, PSAT, or PSPH) can be delivered to the appropriate cells in the subject. Expression of the coding sequence can be directed to any cell in the body of the subject, preferably a cancer cell or adipocyte. This can be achieved, for example, through the use of polymeric, biodegradable microparticle or microcapsule delivery devices known in the art.

The nucleic acid can be introduced into the cells by any means appropriate for the vector employed. Many such methods are well known in the art. Examples of methods of gene delivery include, for example, liposome-mediated transfection, electroporation, calcium phosphate/DEAE dextran methods, gene gun, and microinjection. Delivery of “naked DNA” (i.e., without a delivery vehicle) to an intramuscular, intradermal, or subcutaneous site is another means to achieve in vivo expression. Gene delivery using viral vectors such as adenoviral, retroviral, lentiviral, or adeno-asociated viral vectors can also be used. An ex vivo strategy can also be used for therapeutic applications, as described herein. Ex vivo strategies involve transfecting or transducing cells obtained from the subject with a therapeutic nucleic acid compound. The transfected or transduced cells are then returned to the subject. Such cells act as a source of the therapeutic nucleic acid compound for as long as they survive in the subject.

The therapeutic agent can be packaged alone or in combination with other therapeutic agents as a kit. Additional therapeutic agents that can be used in combination with the therapeutic agents of the invention include chemotherapeutic agents. The kit can include optional components that aid in the administration of the unit dose to subjects, such as vials for reconstituting powder forms, syringes for injection, customized IV delivery systems, or inhalers. Additionally, the unit dose kit can contain instructions for preparation and administration of the compositions. The kit may be manufactured as a single use unit dose for one subject, multiple uses for a particular subject (e.g., at a constant dose or in which the individual compounds may vary in potency as therapy progresses), or the kit may contain multiple doses suitable for administration to multiple subjects (e.g., “bulk packaging”). The kit components may be assembled in cartons, blister packs, bottles, or tubes.

Combination Therapies

Therapeutic compounds that inhibit the polypeptides of the invention (e.g., PHGDH, PSAT, or PSPH) can be used alone or in combination with one, two, three, four, or more of the therapeutic agents of the invention or with a known therapeutic agent for the treatment or prevention of a cellular proliferative disorder, such as a chemotherapeutic agent. Chemotherapeutic agents include, e.g., alkylating agents (e.g., busulfan, dacarbazine, ifosfamide, hexamethylmelamine, thiotepa, dacarbazine, lomustine, cyclophosphamide chlorambucil, procarbazine, altretamine, estramustine phosphate, mechlorethamine, streptozocin, temozolomide, and Semustine), platinum agents (e.g., spiroplatin, tetraplatin, ormaplatin, iproplatin, ZD-0473 (AnorMED), oxaliplatin, carboplatin, lobaplatin (Aeterna), satraplatin (Johnson Matthey), BBR-3464 (Hoffmann-La Roche), SM-11355 (Sumitomo), AP-5280 (Access), and cisplatin), antimetabolites (e.g., azacytidine, floxuridine, 2-chlorodeoxyadenosine, 6-mercaptopurine, 6-thioguanine, cytarabine, 2-fluorodeoxy cytidine, methotrexate, tomudex , fludarabine, raltitrexed, trimetrexate, deoxycoformycin, pentostatin, hydroxyurea, decitabine (SuperGen), clofarabine (Bioenvision), irofulven (MGI Pharma), DMDC (Hoffmann-La Roche), ethynylcytidine (Taiho), gemcitabine, and capecitabine), topoisomerase inhibitors (e.g., amsacrine, epirubicin, etoposide, teniposide or mitoxantrone, 7-ethyl-10-hydroxy-camptothecin, dexrazoxanet (TopoTarget), pixantrone (Novuspharma), rebeccamycin analogue (Exelixis), BBR-3576 (Novuspharma), rubitecan (SuperGen), irinotecan (CPT-11), topotecan, exatecan mesylate (Daiichi), quinamed (ChemGenex), gimatecan (Sigma-Tau), diflomotecan (Beaufour-Ipsen), TAS-103 (Taiho), elsamitrucin (Spectrum), J-107088 (Merck & Co), BNP-1350 (BioNumerik), CKD-602 (Chong Kun Dang), KW-2170 (Kyowa Hakko), and hydroxycamptothecin (SN-38)), antitumor antibiotics (e.g., valrubicin, therarubicin, idarubicin, rubidazone, plicamycin, porfiromycin, mitoxantrone (novantrone), amonafide, azonafide, anthrapyrazole, oxantrazole, losoxantrone, MEN-10755 (Menarini), GPX-100 (Gem Pharmaceuticals), epirubicin, mitoxantrone, and doxorubicin), antimitotic agents (e.g., colchicine, vinblastine, vindesine, dolastatin 10 (NCl), rhizoxin (Fujisawa), mivobulin (Warner-Lambert), cemadotin (BASF), RPR 109881A (Aventis), TXD 258 (Aventis), epothilone B (Novartis), T 900607 (Tularik), T 138067 (Tularik), cryptophycin 52 (Eli Lilly), vinflunine (Fabre), auristatin PE (Teikoku Hormone), BMS 247550 (BMS), BMS 184476 (BMS), BMS 188797 (BMS) , taxoprexin (Protarga), SB 408075 (GlaxoSmithKline), vinorelbine, trichostatin A, E7010 (Abbott), PG-TXL (Cell Therapeutics), IDN 5109 (Bayer), A 105972 (Abbott), A 204197 (Abbott), LU 223651 (BASF), D 24851 (ASTAMedica), ER-86526 (Eisai), combretastatin A4 (BMS), isohomohalichondrin-B (PharmaMar), ZD 6126 (AstraZeneca), AZ10992 (Asahi), IDN-5109 (Indena), AVLB (Prescient NeuroPharma), azaepothilone B (BMS), BNP-7787 (BioNumerik), CA-4 prodrug (OXiGENE), dolastatin-10 (NIH), CA-4 (OXiGENE), docetaxel, vincristine, and paclitaxel), aromatase inhibitors (e.g., aminoglutethimide, atamestane (BioMedicines), letrozole, anastrazole, YM-511 (Yamanouchi), formestane, and exemestane), thymidylate synthase inhibitors (e.g., pemetrexed (Eli Lilly), ZD-9331 (BTG), nolatrexed (Eximias), and CoFactor™ (BioKeys)), DNA antagonists (e.g., trabectedin (PharmaMar), glufosfamide (Baxter International), albumin+³²P (Isotope Solutions), thymectacin (NewBiotics), edotreotide (Novartis), mafosfamide (Baxter International), apaziquone (Spectrum Pharmaceuticals), and O⁶-benzylguanine (Paligent)), Farnesyltransferase inhibitors (e.g., arglabin (NuOncology Labs), lonafarnib (Schering-Plough), BAY-43-9006 (Bayer), tipifarnib (Johnson & Johnson), and perillyl alcohol (DOR BioPharma)), pump inhibitors (e.g., CBT-1 (CBA Pharma), tariquidar (Xenova), MS-209 (Schering AG), zosuquidar trihydrochloride (Eli Lilly), biricodar dicitrate (Vertex)), histone acetyltransferase inhibitors (e.g., tacedinaline (Pfizer), SAHA (Aton Pharma), MS-275 (Schering AG), pivaloyloxymethyl butyrate (Titan), depsipeptide (Fujisawa)), metalloproteinase inhibitors (e.g., Neovastat (Aeterna Laboratories), marimastat (British Biotech), CMT-3 (CollaGenex), BMS-275291 (Celltech)), Ribonucleoside reductase inhibitors (e.g., gallium maltolate (Titan), triapine (Vion), tezacitabine (Aventis), didox (Molecules for Health)), TNFa agonists/antagonists (e.g., virulizin (Lorus Therapeutics), CDC-394 (Celgene), and revlimid (Celgene)), Endothelin A receptor antagonists (e.g., atrasentan (Abbott), ZD-4054 (AstraZeneca), and YM-598 (Yamanouchi)), Retinoic acid receptor agonists (e.g., fenretinide (Johnson & Johnson), LGD-1550 (Ligand), and alitretinoin (Ligand)), Immuno-modulators (e.g., interferon, oncophage (Antigenics), GMK (Progenies), adenocarcinoma vaccine (Biomira), CTP-37 (AVI BioPharma), IRX-2 (Immuno-Rx), PEP-005 (Peplin Biotech), synchrovax vaccines (CTL Immuno), melanoma vaccine (CTL Immuno), p21 RAS vaccine (GemVax), dexosome therapy (Anosys), pentrix (Australian Cancer Technology), ISF-154 (Tragen), cancer vaccine (Intercell), norelin (Biostar), BLP-25 (Biomira), MGV (Progenies), B-alethine (Dovetail), and CLL therapy (Vasogen)), hormonal and antihormonal agents (e.g., estrogens, conjugated estrogens, ethinyl estradiol, chlortrianisen, idenestrol, hydroxyprogesterone caproate, medroxyprogesterone, testosterone, testosterone propionate; fluoxymesterone, methyltestosterone, diethylstilbestrol, megestrol, bicalutamide, flutamide, nilutamide, dexamethasone , prednisone, methylprednisolone, prednisolone, aminoglutethimide, leuprolide, octreotide, mitotane, P-04 (Novogen), 2-methoxyestradiol (EntreMed), arzoxifene (Eli Lilly), tamoxifen, toremofine, goserelin, Leuporelin, and bicalutamide), photodynamic agents (e.g., talaporfin (Light Sciences), Theralux (Theratechnologies), motexafin gadolinium (Pharmacyclics), Pd-bacteriopheophorbide (Yeda), lutetium texaphyrin (Pharmacyclics), and hypericin), and kinase inhibitors (e.g., imatinib (Novartis), leflunomide (Sugen/Pharmacia), ZD1839 (AstraZeneca), erlotinib (Oncogene Science), canertinib (Pfizer), squalamine (Genaera), SU5416 (Pharmacia), SU6668 (Pharmacia), ZD4190 (AstraZeneca), ZD6474 (AstraZeneca), vatalanib (Novartis), PKI166 (Novartis), GW2016 (GlaxoSmithKline), EKB-509 (Wyeth), trastuzumab (Genentech), OSI-774 (Tarceva™), CI-1033 (Pfizer), SU11248 (Pharmacia), RH3 (York Medical), genistein, radicinol, EKB-569 (Wyeth), kahalide F (PharmaMar), CEP-701 (Cephalon), CEP-751 (Cephalon), MLN518 (Millenium), PKC412 (Novartis), phenoxodiol (Novogen), C225 (ImClone), rhu-Mab (Genentech), MDX-H210 (Medarex), 2C4 (Genentech), MDX-447 (Medarex), ABX-EGF (Abgenix), IMC-1C11 (ImClone), tyrphostins, gefitinib (Iressa), PTK787 (Novartis), EMD 72000 (Merck), Emodin, and Radicinol).

Other chemotherapeutic agents include SR-27897 (CCK A inhibitor, Sanofi-Synthelabo), tocladesine (cyclic AMP agonist, Ribapharm), alvocidib (CDK inhibitor, Aventis), CV-247 (COX-2 inhibitor, Ivy Medical), P54 (COX-2 inhibitor, Phytopharm), CapCell™ (CYP450 stimulant, Bavarian Nordic), GCS-100 (gal3 antagonist, GlycoGenesys), G17DT immunogen (gastrin inhibitor, Aphton), efaproxiral (oxygenator, Allos Therapeutics), PI-88 (heparanase inhibitor, Progen), tesmilifene (histamine antagonist, YM BioSciences), histamine (histamine H2 receptor agonist, Maxim), tiazofurin (IMPDH inhibitor, Ribapharm), cilengitide (integrin antagonist, Merck KGaA), SR-31747 (IL-1 antagonist, Sanofi-Synthelabo), CCI-779 (mTOR kinase inhibitor, Wyeth), exisulind (PDE V inhibitor, Cell Pathways), CP-461 (PDE V inhibitor, Cell Pathways), AG-2037 (GART inhibitor, Pfizer), WX-UKI (plasminogen activator inhibitor, Wilex), PBI-1402 (PMN stimulant, ProMetic LifeSciences), bortezomib (proteasome inhibitor, Millennium), SRL-172 (T cell stimulant, SR Pharma), TLK-286 (glutathione S transferase inhibitor, Telik), PT-100 (growth factor agonist, Point Therapeutics), midostaurin (PKC inhibitor, Novartis), bryostatin-1 (PKC stimulant, GPC Biotech), CDA-II (apoptosis promotor, Everlife), SDX-101 (apoptosis promotor, Salmedix), rituximab (CD20 antibody, Genentech, carmustine, mitoxantrone, bleomycin, absinthin, chrysophanic acid, cesium oxides, ceflatonin (apoptosis promotor, ChemGenex), BCX-1777 (PNP inhibitor, BioCryst), ranpinase (ribonuclease stimulant, Alfacell), galarubicin (RNA synthesis inhibitor, Dong-A), tirapazamine (reducing agent, SRI International), N-acetylcysteine (reducing agent, Zambon), R-flurbiprofen (NF-kappaB inhibitor, Encore), 3CPA (NF-kappaB inhibitor, Active Biotech), seocalcitol (vitamin D receptor agonist, Leo), 131-I-TM-601 (DNA antagonist, TransMolecular), eflornithine (ODC inhibitor , ILEX Oncology), minodronic acid (osteoclast inhibitor, Yamanouchi), indisulam (p53 stimulant, Eisai), aplidine (PPT inhibitor, PharmaMar), gemtuzumab (CD33 antibody, Wyeth Ayerst), PG2 (hematopoiesis enhancer, Pharmagenesis), Immunol™ (triclosan oral rinse, Endo), triacetyluridine (uridine prodrug , Wellstat), SN-4071 (sarcoma agent, Signature BioScience), TransMID-107™ (immunotoxin, KS Biomedix), PCK-3145 (apoptosis promotor, Procyon), doranidazole (apoptosis promotor, Pola), CHS-828 (cytotoxic agent, Leo), trans-retinoic acid (differentiator, NIH), MX6 (apoptosis promotor, MAXIA), apomine (apoptosis promotor, ILEX Oncology), urocidin (apoptosis promotor, Bioniche), Ro-31-7453 (apoptosis promotor, La Roche), brostallicin (apoptosis promotor, Pharmacia), β-lapachone, gelonin, cafestol, kahweol, caffeic acid, and Tyrphostin AG. The invention may also use analogs of any of these agents (e.g., analogs having anticancer activity). Exemplary chemotherapeutic agents are listed in, e.g., U.S. Pat. Nos. 6,864,275 and 6,984,654, hereby incorporated by reference.

Combination therapies may provide a synergistic benefit and can include sequential administration, as well as administration of these therapeutic agents in a substantially simultaneous manner. In one example, substantially simultaneous administration is accomplished, for example, by administering to the subject an inhibitor of PHGDH (e.g., an shRNA) and a second inhibitor in multiple capsules or injections at approximately the same time. The components of the combination therapies, as noted above, can be administered by the same route or by different routes (e.g., via oral administration). In different embodiments, a first inhibitor compound may be administered by orally, while the one or more additional inhibitor compounds may be administered intramuscularly, subcutaneously, topically, or all therapeutic agents may be administered orally or all therapeutic agents may be administered by intravenous injection.

### Subject Monitoring

The diagnostic methods described herein can also be used to monitor the progression of a disorder (e.g., a cellular proliferation disorder) during therapy or to determine the dosages of therapeutic compounds. In one embodiment, the levels of, for example, PHGDH polypeptides are measured repeatedly as a method of diagnosing the disorder and monitoring the treatment or management of the disorder. In order to monitor the progression of the disorder in a subject, subject samples can be obtained at several time points and may then be compared. For example, the diagnostic methods can be used to monitor subjects during chemotherapy. In this example, serum samples from a subject can be obtained before treatment with a chemotherapeutic agent, again during treatment with a chemotherapeutic agent, and again after treatment with a chemotherapeutic agent. In this example, the level of PHGDH in a subject is closely monitored and, if the level of PHGDH begins to increase during therapy, the therapeutic regimen for treatment of the disorder can be modified as determined by the clinician (e.g., the dosage of the therapy may be changed or a different therapeutic may be administered). The monitoring methods of the invention may also be used, for example, in assessing the efficacy of a particular drug or therapy in a subject, determining dosages, or in assessing progression, status, or stage of the infection.

### EXAMPLES

The following examples are intended to illustrate the invention. They are not meant to limit the invention in any way.

### General Procedures

The following general methods, along with other methods known in the art, were used in the experiments described herein.

PHGHD Cloning

Human PHGDH cDNA fragment was isolated with EcoRV and NotI from PHGDH/pSport6 (Openbiosystems MHS1010-73507), and cloned into the blunted BamHI and NotI sites of a pLvx-Tight-Puro (Clontech) tetracycline inducible vector.

Cell Lysis, Western Blot, and Immunohistochemistry Analysis

Exponentially growing cells were first washed with cold PBS and lysed with RIPA buffer (10 mM Tris (7.5), 150 mM NaCl, 1% Nonidet P-40, 1% Deoxycholic acid, 0.1% SDS, and 4 μg/mL each of pepstatin, leupeptin, 4-(2-Aminoethyl)benzenesulfonyl fluoride hydrochloride) and aprotinin, a phosphatase inhibitor cocktail (ThermoScientific) and 1 mM DTT. Lysates were centrifuged at 14,000 rpm at 4° C. for 30 minutes and supernatant retained. Protein concentration was determined with Bradford assay (BioRad). Mouse monoclonal PHGDH antibody was purchased from Santa Cruz (sc-100317) and mouse monoclonal beta actin (abCam ab8226) was used as a loading control. Both mouse anti-PSAT antibody (Novus) and rabbit anti-PSPH antibody (Sigma) were used at dilutions of 1:1000. PHGDH antibody was used at 1:500 dilution and incubated at 4° C. overnight with 5% dry milk in Tris-buffered saline (0.05% Tween). Beta actin antibody was used at a 1:10000 dilution. Secondary antibodies conjugated to Horseradish Peroxidase were used at 1:10000 dilution. Western blots were developed using chemiluminescence. Quantitation was carried out using ImageJ software. For Immunohistochemistry, mouse monoclonal PHGDH antibody was purchased from Santa Cruz (sc-100317) and used at 1:15 dilution. Antibody specificity was first validated using paraffin-embedded cell blocks obtained from shGFP and shPHGDH expressing cell lines. All IHC staining was carried out using a Dako Envision (K4006) IHC kit with hematoxylin nuclear counterstain and 3-3′-Diaminobenzidine [DAB] antibody stain.

Cell Culture

All cell lines, other than the T.T. cell line and all human melanoma cell lines,were obtained from ATCC. HEK293T, SkBr3, MCF7, and T.T. cells were grown DMEM (Mediatech), 10% FBS, and antibiotics (Penicilin/Streptomycin, Invitrogen). H1299 cells were grown in RPMI (Mediatech), 10% FBS, and antibiotics. All human melanoma cell lines were cultured as known in the art in RPMI (Mediatech) with 10% FBS and antibiotics. BT20 cells were cultured in MEM (Mediatech), 10% FBS, and antibiotics. Early passage MCF-10a cells were cultured according to a protocol using DMEM/F12(Mediatech), 5% Horse Serum, antibiotics supplemented with Insulin, EGF, Hydrocortisone, and Cholera Toxin (Debnath et al., Methods 30, 256-268 (2003)). Growth media contained the standard concentrations of glutamine but was not supplemented with additional glutamine.

NMR Sample Preparation, Spectroscopy, and Data Analysis

10⁸ exponentially growing HEK293T, H1299 and MCF-10a cells growing in basal growth media with dialyzed serum were harvested and metabolites were extracted in 50 mL of 80% Methanol (v/v) at dry ice temperatures. Cells were incubated with [U¹³C]-glucose (Cambridge Isotope Laboratories) replaced at 25 mM and incubated 24 hrs prior to harvesting. Fresh media were added 2 hours prior to the experiment. Lysates were centrifuged at 10,000g for 30 minutes at 4° C. and supernatant was stored. Methanol was first evaporated at cold temperature under vacuum with rotational evaporation and samples were subsequently lyophilized. Samples were prepared for NMR spectroscopy by resuspending the lyophilized material in 700 μl of sample buffer, containing 50 mM NaPO₄ (pH=7.0) and 2 mM DSS (as an internal standard and chemical shift reference). The samples were immediately transferred into 5 mm, 7″ NMR tubes (Wilmad lab glass) for data acquisition.

All NMR spectra were acquired on a Bruker 500 MHz spectrometer (Bruker, Inc., Billerica, Mass.) using a 5 mm triple resonance (H, C, N) Cryoprobe. The sample temperature was 25° C. for all samples. Two-dimensional 1H-13C HSQC spectra with sensitivity enhancement were acquired with spectral widths of 12000 Hz and 9048 Hz in the direct and indirect dimensions, respectively. 1024 complex data points were acquired in the direct dimension, and 256 complex points were acquired in the indirect dimension in a linear fashion, with a subsequent 256 complex points being acquired with a non-uniform random sampling scheme. The total acquisition time for the indirect dimension was 113 milliseconds. 64 dummy scans were collected prior to the first increment, and 16 scans were acquired per increment.

The resulting HSQC spectra were processed using NMRpipe. A zero order phase correction in the directly detected dimension was used. Spectra were then extracted in ascii format and peaks from 0-10 ppm in the proton dimension and 20-160 ppm in the carbon dimension were considered. This resulted in 1704 data points in the direct dimension and 423 data points in the indirectly detected dimension. The resulting intensities at each data point were then binned using an eight-fold reduction in the proton dimension and a two-fold reduction in the carbon dimension. The intensities at each point in the resulting 213×206 lattice were then computed and a baseline value of 5e6 was defined that corresponded to a value above the signal to noise level and each bin exhibiting sum intensity less than that of the baseline was set to the baseline. Bins in the region of the spectra containing the water line (4.60-4.75 ppm) were omitted. The resulting bins that displayed at least a two-fold increase in the intensity relative to the noise level were considered. Individual metabolite assignments were carried out using the Human Metabolome Database (HMDB). Computer code was written in the PERL interpreting language. Zscores (i.e., intensities in standard units) were computed in Matlab. ¹³C Glycine peaks were integrated separately using the Sparky software package (www.cgl.ucsfledu/home/sparky/). Peak intensities were computed using gaussian integration and error bars obtained from RMS residuals.

Targeted Liquid-Chromatography Mass Spectrometry (LC/MS)

10⁶ cells exponentially growing in basal media with dialyzed serum were harvested in 3 mL 80% v/v methanol at dry ice temperatures. Fresh media was added 24 hours and 2 hours prior to the experiment. Insoluble material in lysates was centrifuged at 4000RPM for 15 minutes and resulting supernatant was evaporated using a refrigerated speed-vac. Samples were resuspended using 20 μL HPLC grade water for mass spectrometry. 10 μL were injected and analyzed using a 5500 QTRAP triple quadrupole mass spectrometer (AB/MDS Sciex) coupled to a Prominence UFLC HPLC system (Shimadzu) via selected reaction monitoring (SRM) of a total of 249 endogenous water soluble metabolites for analyses of samples. Some metabolites were targeted in both positive and negative ion mode for a total of 298 SRM transitions. ESI voltage was 5000V in positive ion mode and −4500V in negative ion mode. The dwell time was 5 ms per SRM transition and the total cycle time was 2.09 seconds. Samples were delivered to the MS via normal phase chromatography using a 2.0 mm i d×15 cm Luna NH2 HILIC column (Phenomenex) at 285 μL/min. Gradients were run starting from 85% buffer B (HPLC grade acetonitrile) to 42% B from 0-5 minutes; 42% B to 0% B from 5-16 minutes; 0% B was held from 16-24 minutes; 0% B to 85% B from 24-25 minutes; 85% B was held for 7 minutes to re-equilibrate the column. Buffer A was comprised of 20 mM ammonium hydroxide/20 mM ammonium acetate in 95:5 water : acetonitrile. Peak areas from the total ion current for each metabolite SRM transition were integrated using MultiQuant v1.1 software (Applied Biosystems). Glucose-13C labeled samples were run with 249 total SRM transitions (40 in positive ion mode and 209 in negative ion mode) with a total cycle time of 0.464 seconds.

Isotope Labeling and Kinetic Profiling

Basal media using dialyzed serum without glucose was supplemented with [U¹³C]-glucose (Cambridge Isotope Laboratories) to a concentration equivalent to the concentration suggested by ATCC protocol. Fresh media was added two hours prior to the kinetics experiment. Media was replaced by equivalent [U¹³C]-glucose labeled media and cells quickly harvested at given time points using the above-mentioned protocol. Steady-state [U¹³C]-glucose labeling involved labeling cells for 12 hours prior to metabolite extraction. Samples were prepared as described above. Data analysis was performed in Matlab.

Gas-Chromatography Mass Spectrometry (GC/MS)

Cells were cultured in 6-well plates before replacing medium with DMEM containing 10% dialyzed FBS and either [U-¹³C]glucose+unlabeled glutamine or [α-¹⁵N]glutamine and unlabeled glucose. After 24 hours, cells were rinsed with 1 ml ice cold PBS and quenched with 0.4 ml ice cold methanol. An equal volume of water was added, and cells were collected in tubes by scraping with a pipette. One volume of ice cold chloroform was added to each tube, and the extracts were vortexed at 4° C. for 30 minutes. Samples were centrifuged at 14,000 g for 5 minutes, and the aqueous phase was transferred to a new tube for evaporation under nitrogen airflow.

Derivatization and GC/MS measurements

A two-step derivitization method was used as described in Antoniewicz et al. (Analytical Chemistry 79, 7554-7559 (2007)). Dried polar metabolites were dissolved in 20 μl of 2% methoxyamine hydrochloride in pyridine (Pierce) and held at 37° C. for 1.5 hours. After dissolution and reaction, tert-butyldimethylsilyl (TBDMS) derivatization was initiated by adding 30 μl N-methyl-N- (tert-butyldimethylsilyl)trifluoroacetamide MBTSTFA+1% tert-butyldimethylchlorosilane TBDMCS (Pierce) and incubating at 55° C. for 60 minutes. Gas chromatography/mass spectrometry (GC/MS) analysis was performed using an Agilent 6890 GC equipped with a 30 m DB-35MS capillary column connected to an Agilent 5975B MS operating under electron impact (EI) ionization at 70 eV. One μl of sample was injected in splitless mode at 270° C., using helium as the carrier gas at a flow rate of 1 ml min⁻¹. The GC oven temperature was held at 100° C. for 3 min and increased to 300° C. at 3.5° min⁻¹. The MS source and quadrupole were held at 230° C. and 150° C., respectively, and the detector recorded ion abundance in the range of 100-600 m/z. Mass isotopomer distributions (MIDs) for serine and glycine were determined by integrating ion fragments of 390-398 m/z and 246-252 m/z, respectively. MIDs were corrected for natural isotope abundance using algorithms adapted from Fernandez et al. (J Mass Spectrom 31, 255-62 (1996)).

Analysis of Somatic Copy Number Alterations Of PHGDH

Data processed in Matlab across 3131 total samples and 150 melanoma samples from the Broad Institute as previously compiled (Beroukhim et al., Nature 463, 899-905 (2010)). Heatmaps were generated in Matlab by first sorting copy number intensity at the coding region of PHGDH. False discovery rates (q-values) on chromosome 1p were computed using a background model previously developed and plotted in Matlab. q-values for candidate oncogenes were reported as in Beroukhim et al. (Nature 463, 899-905 (2010)).

Cell Proliferation Assays

Lentiviral infection and puromycin selection was carried out under established protocols. After puromycin selection, control and knockdown cells were plated at equal densities at initial densities were normalized to the intrinsic growth rate of each cell line and seeded cells allowed to grow for three days prior to counting. Cell numbers were counted on the final day using an automated cell counter (Cellometer Auto T4, Nexcelom Bioscience) with custom morphological parameters set for each cell line. Error bars were reported using error propagation from the standard deviation of three experiments.

3-Dimensional Culture and Confocal Microscopy

To generate acini, cells were grown in reconstituted basement membrane (Matrigel) as known in the art (see, e.g., the protocol available at http://brugge.med.harvard.edu/). The overlay media was changed every four days and a given concentration of doxycycline (Sigma) was added where indicated. Acini were fixed between days 25 and 28 and immunofluorescence analyses of acini was performed as described in the art. The following primary antibodies were used for immunofluorescence: cleaved caspase-3 (#9661, Cell Signaling Technology) and laminin-5 (mab19562, Millipore, Billerica, Mass.). The golgi apparatus was detected combining antibodies to the golgi proteins GM130 (610823, BD Biosciences) and Golgin-84 (51-9001984, BD Biosciences). DAPI (Sigma-Aldrich) was used to counterstain nuclei. For examination of luminal filling, acini were imaged using confocal microscopy to visualize the centre of each structure, and then were scored as clear (˜90-100% clear), mostly clear (˜50-90% clear), mostly filled (˜10-50% clear), or clear (˜0-10% clear).

Fluorescence In-situ Hybridization (FISH).

Cultured cell lines were harvested at 75% confluence and metaphase chromosome spreads were produced using conventional cytogenetic methods. Human melanoma tissue arrays were first heated to remove paraffin. Slides were aged overnight at 37° C., dehydrated by successive two minute washes with 70%, 80%, 90% and 100% ethanol, air-dried and then hybridized to DNA probes as described below. The following DNA probes were co-hybridized: RP11-22F13 (labeled in SpectrumGreen), which maps to 1p12 and includes PHGDH, and the D1Z5 alpha-satellite probe (SpectrumOrange; Abbott Molecular, Inc.), which maps to 1p11.1-q11.1. The RP11-22F13 BAC clone was obtained from CHORI (www.chori.org), direct-labeled using nick translation, and precipitated using standard protocols. Final probe concentration was 100 ng/ul. The final concentration used for the commercial probes followed manufacturer's recommendations. The tissue sections and probes were co-denatured at 80° C. for 5 min, hybridized at least 16 hrs at 37° C. in a darkened humid chamber, washed in 2×SSC at 70° C. for 10 min, rinsed in room temperature 2×SSC, and counterstained with DAPI (4′,6-diamidino-2-phenylindole, Abbott Molecular/Vysis, Inc.). Slides were imaged using an Olympus BX51 fluorescence microscope. Individual images were captured using an Applied Imaging system running CytoVision Genus version 3.92.

Human Tumor Samples And Data Analysis

Human breast cancer patient samples were obtained from the Harvard SPORE breast tissue repository collected under DF/HCC IRB protocol #93-085. Tumor and patient characteristics, tissue microarray construction, and gene expression profiles were known. Histological diagnosis and comparison with clinical parameters was based on established criteria (Richardson et al., Cancer Cell 9, 121-132 (2006)). Human melanoma patient samples were obtained from the Yale SPORE skin cancer program and tissue microarray construction was previously reported (Hoek et al., Cancer Research 64, 5270-5282 (2004)). Histological diagnosis was based on established criteria. All bioinformatics data from human breast cancer microarrays were obtained from Oncomine using established statistics (Rhodes et al., Neoplasia 6, 1-6 (2004)).

### Example 1

### Rearrangement of Glycolytic Flux in Proliferating Cells

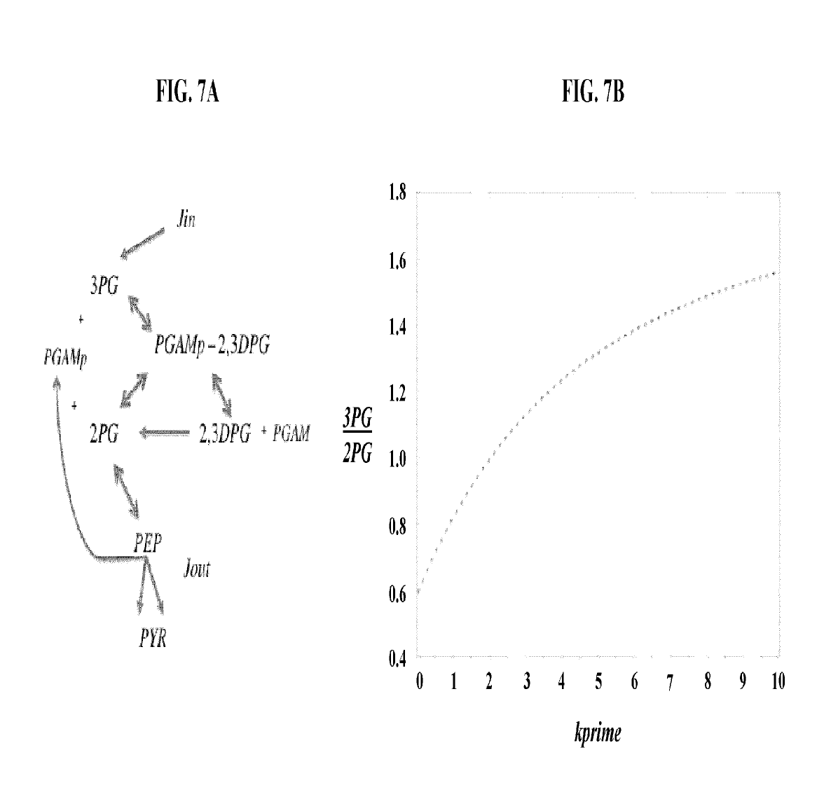

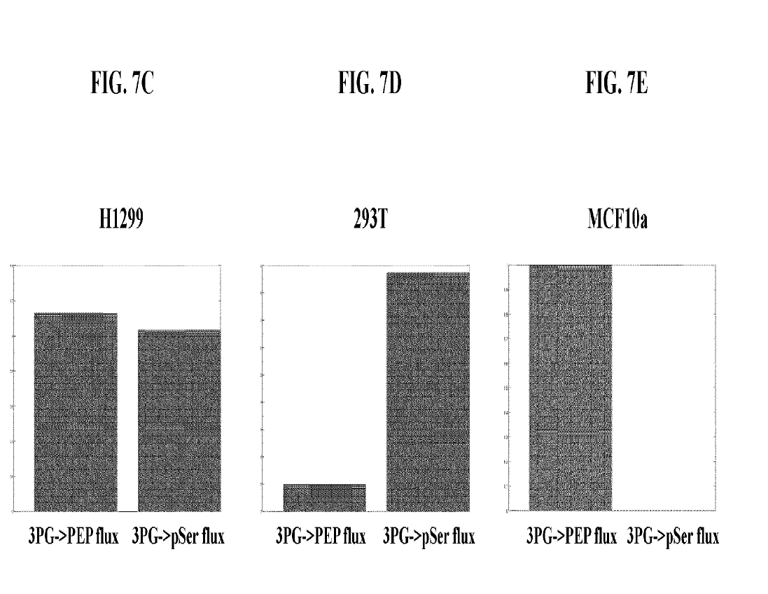

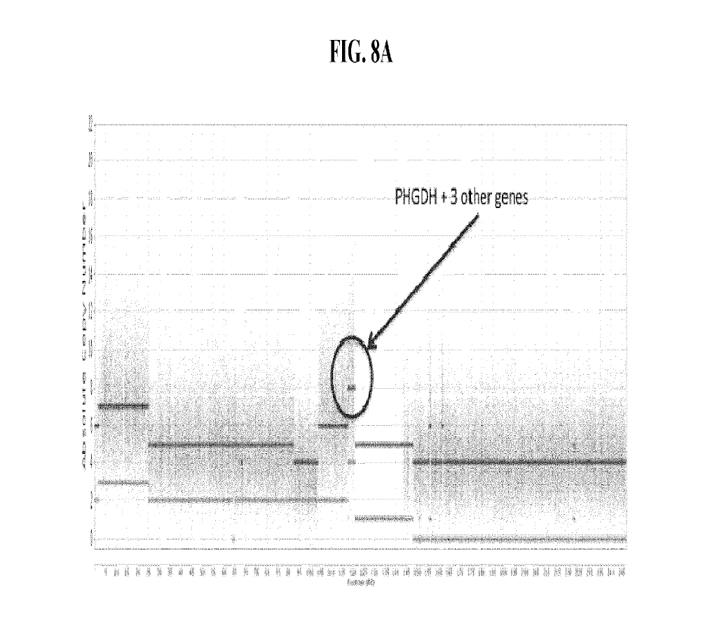

Metabolic profiling of cells where PK-M2 activity has been decreased by RNAi or by increased phosphotyrosine activity by drug treatment shows a large increase in the metabolite 2,3-diphosphoglycerate. This change does not conform to known models of glycolysis. It does, however, imply a novel regulation of the glycolytic pathway from 3-phosphoglycerate (3-PG) through pyruvate that has not previously been described (FIG. 7A). A computer model considering the reported alternative glycolytic pathway depicted in FIG. 7A was constructed. The model includes an incoming flux, Jᵢₙ, originating from the upstream glycolysis pathway resulting in the production 1,3-diphosphoglycerate and an output flux, Jₒᵤₜ, which takes into account the generation of pyruvate.

Michaelis-Menten kinetics for each enzymatic step in the pathway were used. Equations of the form

$$\frac{d {x}^{i}}{d t} = \frac{{v}_{max} {x}^{i}}{{K}_{M} + {x}^{i}}$$

were used.

Modeling this regulation using computer simulations (FIG. 7B) suggests that 3-PG should accumulate in the presence of decreased PK-M2 activity, as would be expected in proliferating cells. FIG. 7B reports the relative levels of 3-PG, the substrate of the enzyme encoding phosphoglycerate dehydrogenase (PHGDH) obtained from the simulation. Numerical solutions to the set of seven differential equations were obtained using a Runge-Kutta fourth-order method implemented in MATLAB. Simulations were carried out for a time sufficient to reach steady state. Parameter values corresponding to typical values known to one of skill in the art were considered. Results in FIG. 7B are robust to large variations in all parameter values, as suggested from a Monte-Carlo sampling of 10,000 random parameter sets.

In agreement with this model, a major portion of the glucose taken up by cells is converted to serine under conditions favoring cell proliferation. We observed that between 40% and 90% of the total flux of glucose that is converted to 3-PG enters the serine biosynthesis pathway (FIGS. 7C and 7D), as determined by NMR spectroscopy on whole cell extracts of different cancer cell lines using ¹³C glucose isotopic tracing. Conversely, we did not detect ¹³C-labeled intermediates in the serine biosynthesis pathway under conditions favoring cell quiescence.

Approximately 10⁸ exponentially-growing, sub-confluent H 1299 and HEK293T adherent cells were harvested. H1299 cells (FIG. 7C) were grown in RPMI media with 10% dialyzed FBS, antibiotics, and 2 mM glutamine. HEK293T cells (FIG. 7D) were grown in DMEM, 10% dialyzed FBS, and antibiotics. MCF10a cells (FIG. 7E) were grown in DMEM/F12 media, 5% horse serum, 1:100 penicillin/streptomycin, EGF (20 ng/ml), insulin (10 μg/ml), hydrocortisone (0.5 mg/ml), and cholera toxin (100 ng/ml). Metabolites were extracted using a 80:20 methanol:water mixture at -80° C. The purified metabolite extract was dried to completion and the resulting solid was resuspended in an NMR buffer consisting of sodium phosphate buffer (pH 7.0), D20, and 50 mM DSS as an internal standard. [¹H,¹³C] Heteronuclear single quantum correlation spectra (HSQC) using a uniform excitation over the entire frequency spectrum of ¹³C resonances were obtained. Such methods were performed to allow for quantitative comparison of different compounds in the metabolite mixture. Assignments of compounds in the spectra were determined using an HSQC reference database obtained from the Human Metabolite Database. Phosphoserine, glycine, and potential serine compounds were identified in the mixture. Flux ratios were obtained by quantifying the relative concentrations and resulting chemical potentials using the following equation:

where Δμ is the chemical

$$Δ μ = Δ {μ}^{0} + RT ln ( \frac{{C}_{1}}{{C}_{2}} )$$

potential, Δμ⁰ is the reference chemical potential, Cᵢ are the concentration at the different points in the pathway, and RT is the thermal energy scale.

### Example 2

### Glucose Metabolism Studies

To better understand the diversity of glucose metabolism, sensitivity-enhanced NMR based 2-dimensional heteronuclear single quantum correlation spectroscopy (HSQC) was used to quantify steady state levels of glucose-derived metabolites in HEK293T cells following 24 hours of labeling with [U-¹³C]-glucose (Bodenhausen et al., Chemical Physics Letters 69, 185-189 (1980)). The spectra were discretized and the intensities of each resulting bin were computed (FIG. 12A). Consistent with previous descriptions of glucose metabolism in cancer cells, two of the four highest intensity bins contained lactate peaks (FIG. 12A). Further, a bin containing ¹³C-glycine was nearly as abundant as that containing ¹³C-lactate (FIG. 12A).

To determine whether this result was general to all cultured cells as has been suggested (Bismut et al., Biochemical Journal 308, 761-767 (1995); Snell et al., Biochemical Journal 245, 609-612 (1987); and Kit, Cancer Research 15, 715-718 (1955)), a [U-¹³C] glucose HSQC experiment was conducted in two other exponentially growing cell lines: H1299 (an epithelial lung cancer cell line) and MCF-10a (a non-tumorigenic mammary epithelial cell line). In H1299 cells, smaller relative quantities of ¹³C labeled glycine (FIG. 12B) were detected; in MCF-10a cells, no ¹³C labeled glycine was observed (FIG. 12B). Together, these data indicate that cell lines display variability in glucose metabolism with differences in relative flux of glucose to glycine.

To further investigate glucose metabolism in cells, the time course of conversion of [U-¹³C] glucose to other metabolites was monitored using targeted liquid chromatography/mass spectrometry (LC/MS) (Lu et al., Journal of Chromatography B-Analytical Technologies in the Biomedical and Life Sciences 871, 236-242 (2008)) in HEK293T cells. ¹³C-labeled glucose incorporation into thirteen metabolites, in multiple pathways, was detected over the 30-minute time course (FIG. 12D). The time required for labeled carbon to reach steady state in a pathway is a direct measurement of pathway flux. The data in FIG. 12E reveal that ¹³C incorporation into pSER (¹³C-pSER) reaches steady state at a time scale comparable to the time for phosphoenolpyruvate (PEP) to reach steady state, suggesting that the relative fluxes are comparable. The ¹³C-pSER labeling accompanied labeling of serine and labeling of serine was also confirmed using GC/MS by measuring pool sizes of incorporation of [α-¹⁵N] glutamine into amino acids. These data are in agreement with NMR experiments suggesting that a substantial fraction of glucose is diverted from 3PG into the serine and glycine biosynthetic pathway in these cells.

To measure the total amount of glucose-derived serine, cultured HEK293T cells and uniformly labeled ¹³C glucose were used. The metabolites from cell extracts were then analyzed using LC/MS. The total amount of labeled serine was found to be about one half, and this value was commensurate with the relative amount of glucose incorporation into nucleotides and nucleotide intermediates with the remaining fraction coming from other nutrients and salvage pathways (FIG. 12F).

Further, expression of PHGDH was verified by Western blot (FIG. 12G): greater PHGDH protein expression in HEK293T cells were observed compared to levels of expression observed in H1299 and MCF10a cells. Thus, the increased synthesis of glycine from glucose in HEK293T cells is associated with higher PHGDH protein levels and the absence of its detection in MCF10a cells corresponds to approximately 30-fold lower protein expression.

### Example 3

### PHGDH Activity and the Copy Number at the Genomic Locus Containing the PHGDH Gene; shRNA Knockdown Experiments

The selective diversion of glucose metabolism into serine metabolism through PHGDH suggested that selective pressure exists for tumors to increase PHGDH activity. PHGDH activity may be enhanced by increasing the copy number at the genomic locus containing the PHGDH gene. We identified PHGDH in a study of a pooled analysis of somatic copy number alterations (SCNA) as a frequently amplified gene across 3131 cancer samples (Beroukhim et al., Nature 463, 899-905 (2010)). Compared to the false discovery rate (q-value) obtained from the background rate of SCNA in cancer, PHGDH was found in a peak of a region of chromosome 1p (1p12) that exhibits recurring copy number gain in 16% of all cancers. No known oncogenes are contained in the peak region of five genes (PHGDH, REG4, HMGCS2, NBPF7, ADAM30) at this locus. PHGDH is located in one of four peak regions of chromosome 1p (q=1.12e-9) (FIG. 13A, left). Two of the three high-scoring peaks contain the oncogenes MYCLJ at 1p34 (q=1.7e-14) and JUN at 1p32 (q=8.55e-7) (FIG. 13A, left). The copy number intensity of 150 cancers sorted by highest PHGDH copy number (FIG. 13A, middle) was plotted along chromosome 1p showing that most samples containing PHGDH copy number gain have the genomic amplification localized near the 1p12 region. An inspection of the genomic region containing PHGDH (FIG. 13A, right) illustrated the localized, amplification within the coding region of the PHGDH gene. Amplification was found most commonly in melanoma at 40% frequency in a three-gene peak region (q=1.93e-5) with HMGCS2 and REG4. We first examined T.T. cells, an esophageal squamous cell carcinoma cell line that contained a highly focal copy number gain of PHGDH (Beroukhim et al., Nature 463, 899-905 (2010)) as determined by SNP array, and carried out fluorescence in situ hybridization (FISH) to verify copy number gain (FIG. 13B). Focal copy number gain in PHGDH suggested that expression might be important for proliferation in these cells and stable PHGDH knockdown using shRNA reduced the proliferation rate (FIG. 13B). To test whether the decreased proliferation was due to alterations in the ability to utilize the serine biosynthesis pathway, we created cell lines with decreased expression of downstream enzymes PSAT and PSPH and found that shRNA-mediated knockdown of these enzymes resulted in similar decreases in proliferation (FIG. 13B).

As PHGDH amplification in a single tumor type was most commonly found in melanoma, we assessed PHGDH expression and copy number gain in human melanoma tissue samples. Immunohistochemistry (IHC) was used to measure PHGDH expression in a tissue collection of human melanoma and high expression (IHC score >1) was observed in 21% of the samples. We then used FISH to probe relative PHGDH copy number in a subset of 42 of these samples. PHGDH copy number gain was observed in 21 of the 42 samples; however, 16 of these samples also contained an equal increased number of copies of a probe sequence adjacent to the centromere, indicating either polysomy or that the amplified region also contained the pericentromeric region of chromosome 1p. Five tumors exhibited copy number gain with the number of copies greater than the number of pericentromeric probes (FIG. 13C). It was observed that each sample with relative gain had high expression by IHC (FIG. 13C), indicating that PHGDH copy number gain and amplification associates with significant protein overexpression in human melanoma (p=0.0045, Fisher's exact test, two-tailed).

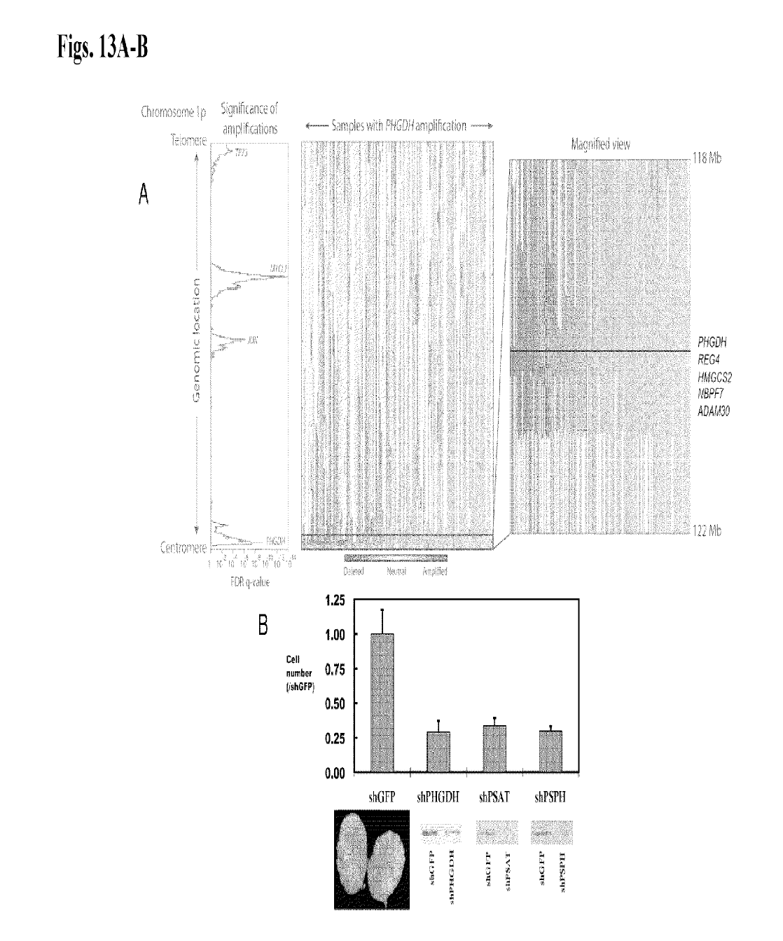

We next investigated whether melanoma cell lines containing PHGDH copy number gain would be sensitive to decreased expression of PHGDH. Three tumor-derived human melanoma cell lines (WM1266-3, Malme-3M, and SK-Mel28) with 1p12 gain were obtained along with two additional melanoma cell lines (Gak, Carney) (Greshock et al., Cancer Research 67, 10173-10180 (2007)). Pairs of cell lines containing shRNA targeting PHGDH and GFP as a control were created for each cell line (FIG. 14A, left). Each of the amplified cell lines showed decreased proliferation in contrast to the non-amplified cell lines that showed no difference in proliferation upon PHGDH knockdown indicating that the growth of the amplified cell lines is differentially sensitive to PHGDH knockdown (FIG. 14A, right). To verify that high expression leads to metabolic flux through the serine pathway, we measured the relative incorporation of ¹³C serine from [U-¹³C] glucose and found that each of the amplified cell lines had appreciable glycolytic flux into serine (FIG. 14B). One cell line that did not contain the amplification, Carney, had high expression of PHGDH and high flux into serine synthesis (FIGS. 14A and B). Previous studies of oncogene addiction have shown that loss of cancer cell proliferation correlates with the presence of a genetic lesion and not with gene expression (Slamon et al., Science 235, 177-182 (1987), and Luo et al., Cell 136, 823-837 (2009)). Consistent with these findings, it was observed that PHGDH knockdown had no effect on growth in Carney cells despite increased serine pathway flux (FIG. 14A).

### Example 4

### shRNA Knockdown Experiments, Serine Pathway Metabolism, and Cancer Cell Growth

The effect of inhibiting genes that encode enzymes outside of glycolysis that divert carbon from 3-PG into the serine biosynthesis pathway (e.g., PHGDH, PSAT, and PSPH) was also studied. 3-PG is oxidized by phosphoglycerate dehydrogenase to form 3-phosphohydroxypyruvate. 3-Phosphohydroxypyruvate is then transaminated to generate phosphoserine. Phosphoserine is desphosphorylated irreversibly to form serine.

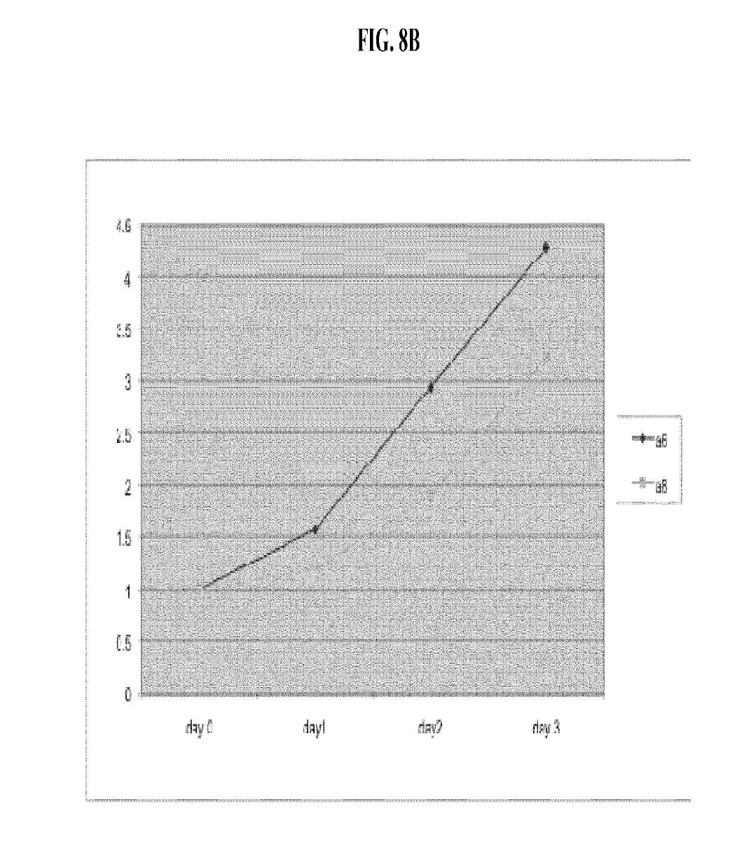

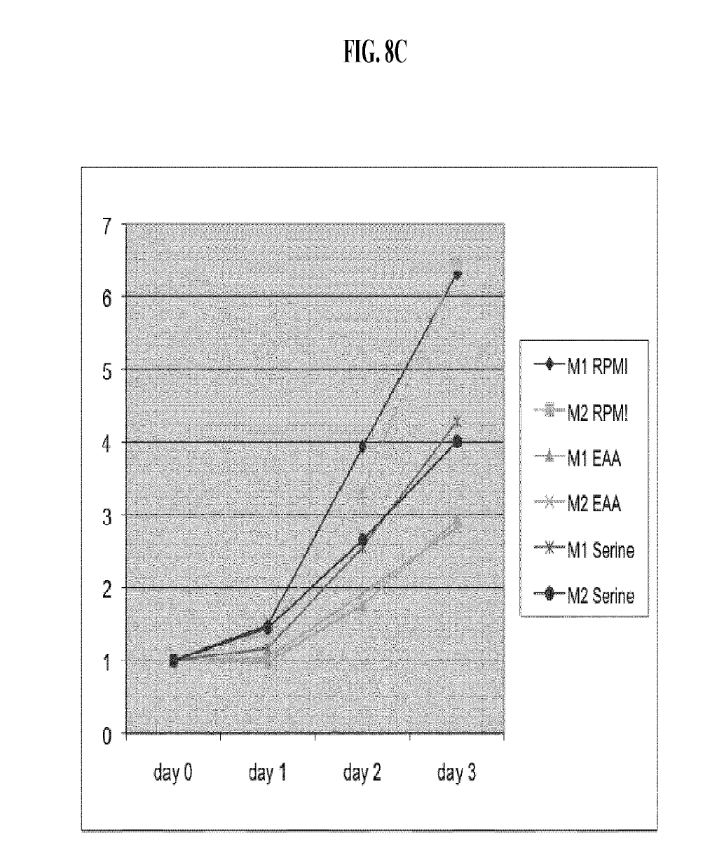

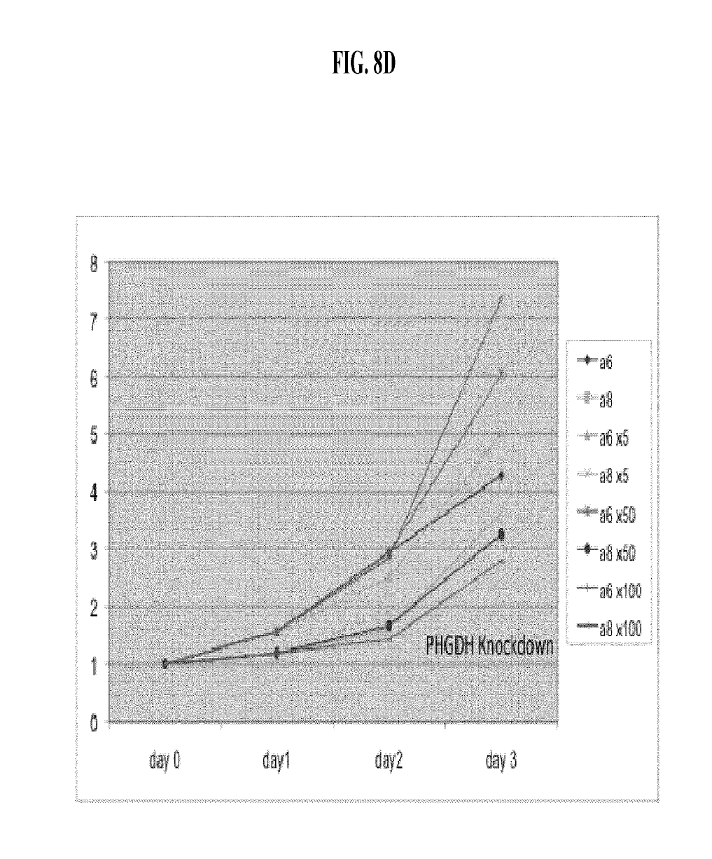

We noted that the locus, 1p12,0 containing PHGDH was included in a focal amplification event without a known oncogenic driver in available databases in certain cell lines. We then considered a human melanoma cell line (Sk-Mel28) that contained a focal amplification of PHGDH resulting in ˜8 copies of the gene (FIG. 8A; data obtained from Sanger Institute Cancer Genome Project Database).

We found that shRNA knockdown of PHGDH significantly inhibited the growth of cancer cells. The following shRNA sequences were used:

(SEQ ID NO: 8)
CCGGAGGTGATAACACAGGGAACATCTCGAGATGTTCCCTGTGTTATCA
CCTTTTTT
Mature Sense for TRCN0000028548:
(SEQ ID NO: 9)
AGGTGATAACACAGGGAACAT
Mature Antisense for TRCN0000028548:
(SEQ ID NO: 10)
ATGTTCCCTGTGTTATCACCT

Particularly, the shRNA inhibited the growth of cells in the cell line that amplified PHGDH (FIG. 8B). For this experiment, shRNA hairpins in lentiviral vectors containing puromycin resistance selection markers were purchased from Open Biosystems. Cells were infected with lentivirus, subjected to selection in growth media supplemented with 2 mg/ml puromycin for three days. After replacing the selection media with regular growth media, ˜50,000 cells were plated in 6-well plates and counted. Cell numbers were obtained using automated Cellometer Auto T4 imaging software from Nexelcom Biosciences. Rate constants for growth of the parental cell line, PHGDH shRNA knockdown 1 cells, and PHGDH knockdown 2 cells were plotted. Western blots of PHGDH protein levels confirmed RNA interference.

FIG. 8C shows that cell growth is enhanced by the addition of exogenous serine. This demonstrates that cells have the ability to use serine from the surrounding media. This ability to take up serine is independent of the expression of PK-M1- or PK-M2-expression in H1299 cells. Cells were grown in RPMI or MEM (supplemented with essential amino acids (Invitrogen), serine, or full media) and 10% FBS. Growth assays were then performed, as described above.

FIG. 8D shows that serine fails to rescue PHGDH knockdown (A8) cells in 5×, 50×, and 100× relative serine concentration with respect to serine concentration in RPMI. Growth assays were then performed, as described above. These findings suggest that cells are dependent on PHGDH for proliferation to perform another function for cells other than serine production.

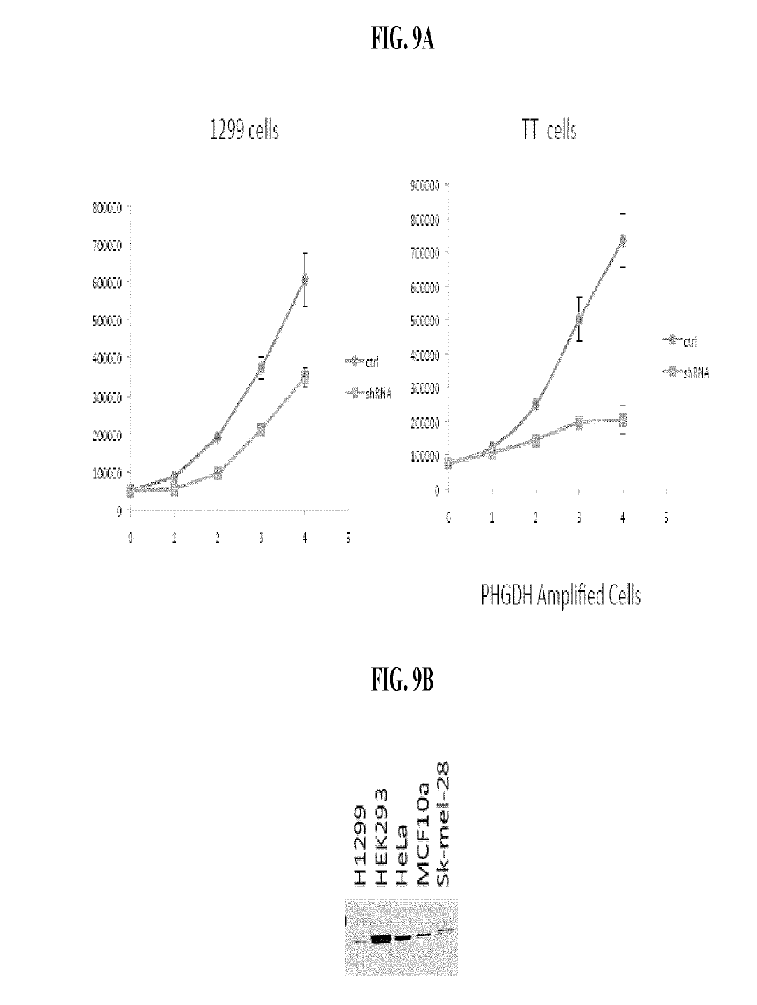

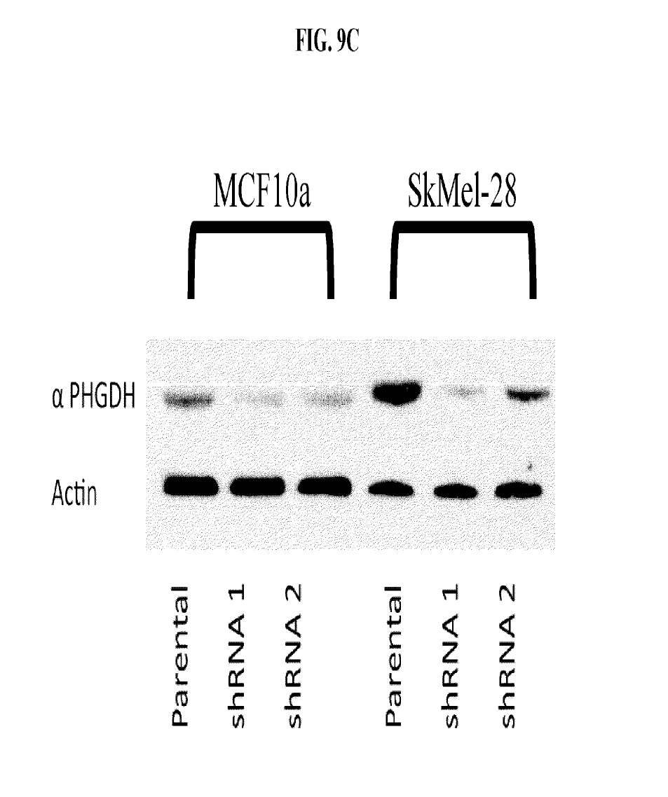

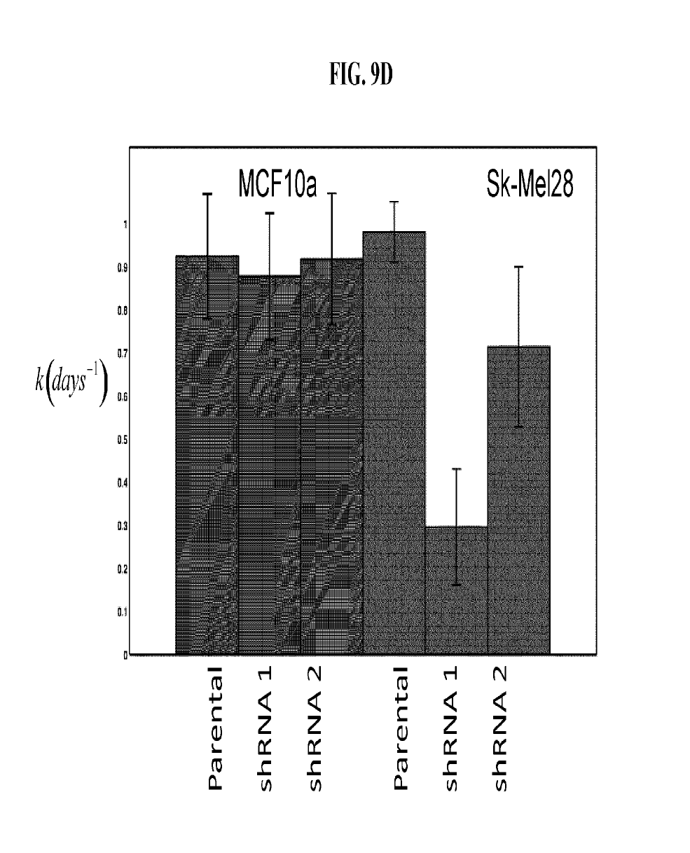

We have also shown the effect of PHGDH RNA interference on cell growth in a cell line that expresses PHGDH, but where the PHGDH gene is not amplified (e.g., H1299 cells) compared with a cell line where the PHGDH gene is amplified (e.g., TT cells) (FIG. 9A). Cells were treated with a control shRNA or a PHGDH-specific shRNA. Western blots of PHGDH protein levels confirmed knockdown of the PHGDH gene in cells treated with PHGDH-specific shRNA (data not shown). The results show that cells with PHGDH gene amplification (TT cells) were more sensitive to PHGDH knockdown than cells that express PHGDH (H1299 cells), but where the PHGDH gene is not amplified.

PHGDH expression alone does not predict which cell lines are sensitive to PHGDH knockdown. A Western blot to determine the expression of PHGDH across several different cell lines shows that many cell lines express PHGDH (FIG. 9B). H1299 cells express PHGDH (FIG. 9B), but are insensitive to PHGDH knockdown (FIG. 9A). Similarly, MCF10a cells and Sk-Mel-28 cells express PHGDH (FIG. 9C). PHGDH expression can be knocked down to different degrees in these cell lines using lentiviral shRNA hairpins (FIG. 9C), as described above. (Parental cells shown in FIGS. 9C and 9D are cells without lentiviral-mediated shRNA knockdown of PHGDH.) Growth of Sk-Mel-28 cells, which harbor PHGDH gene amplification (FIG. 8A), is sensitive to PHGDH knockdown in a dose-dependent fashion, while MCF10a cells grow regardless of PHGDH knockdown (FIG. 9D). Therefore, expression alone does not determine whether cells will be sensitive to PHGDH inhibition. In addition, these results demonstrate that PHGDH gene amplification is a predictive tool to determine response to PHGDH inhibition.

The effect on metabolism by knockdown of PHGDH to levels that impair proliferation was also studied. Metabolomics was carried out on SK-Mel28 cells using targeted LC/MS to profile metabolite levels with or without knockdown of PHGDH. Consistent with affecting the activity of glucose flux into serine metabolism, PHGDH knockdown reduced pSER levels in Sk-Mel28 cells (FIG. 14C) and globally altered metabolite levels including the levels of many intermediates in glycolysis (FIG. 14D). Increased levels of metabolites in glycolysis near the point of diversion into serine metabolism were observed (FIG. 14E) confirming that the level of PHGDH expression alters glucose metabolism in SkMel-28 cells by modulating the entry of glycolytic metabolites into serine metabolism.

### Example 5

### Production of NADPH by Phosphoglycerate Dehydrogenase

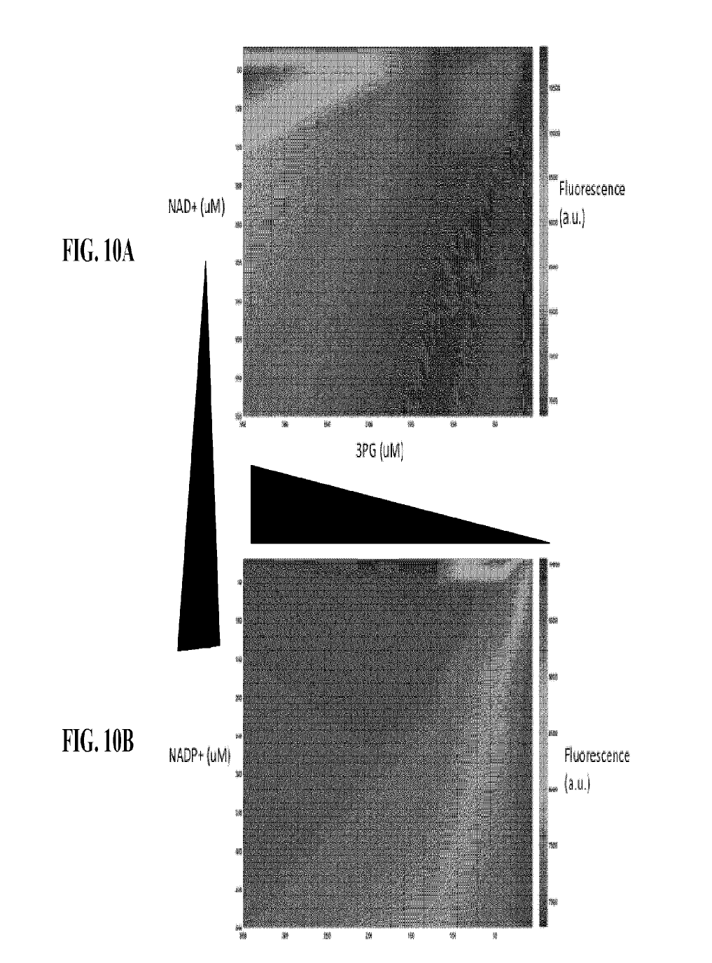

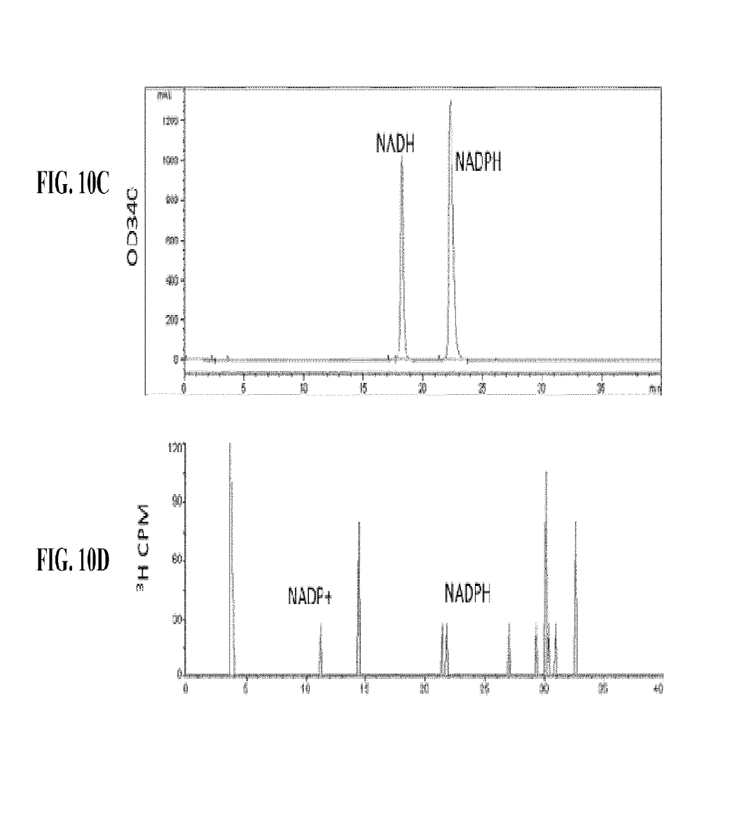

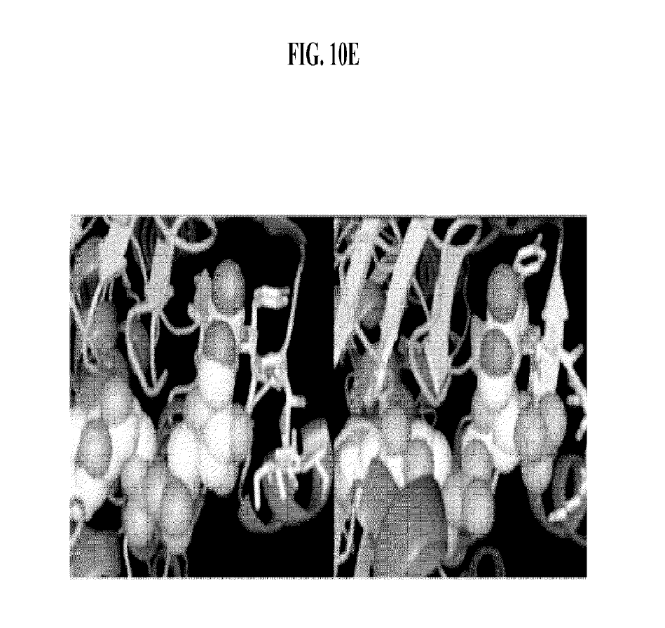

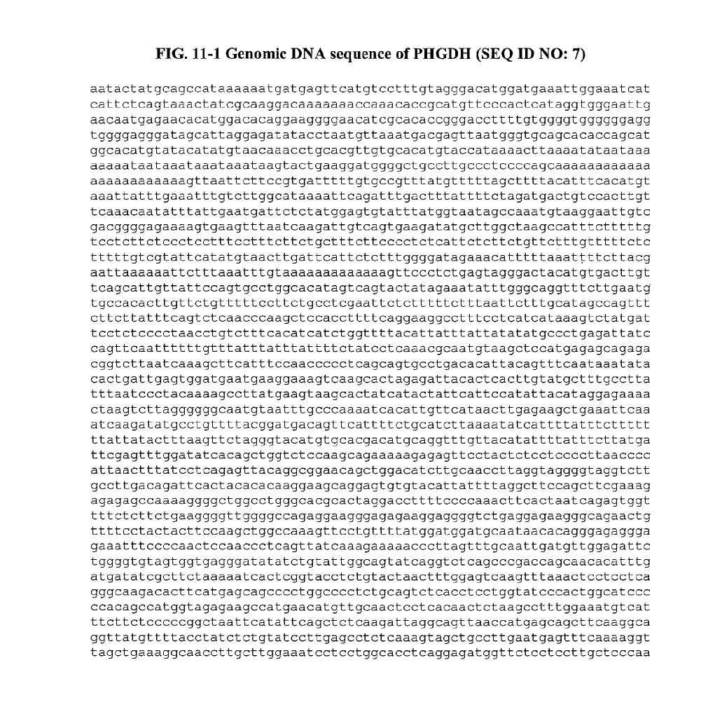

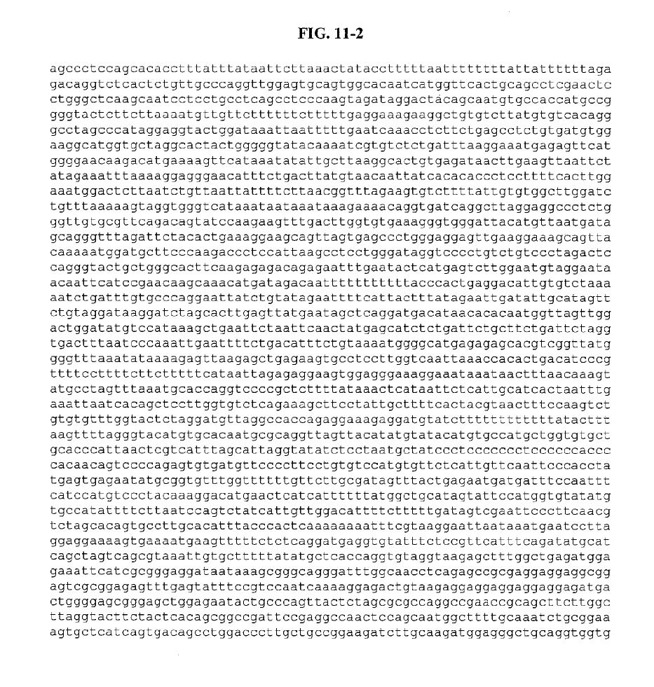

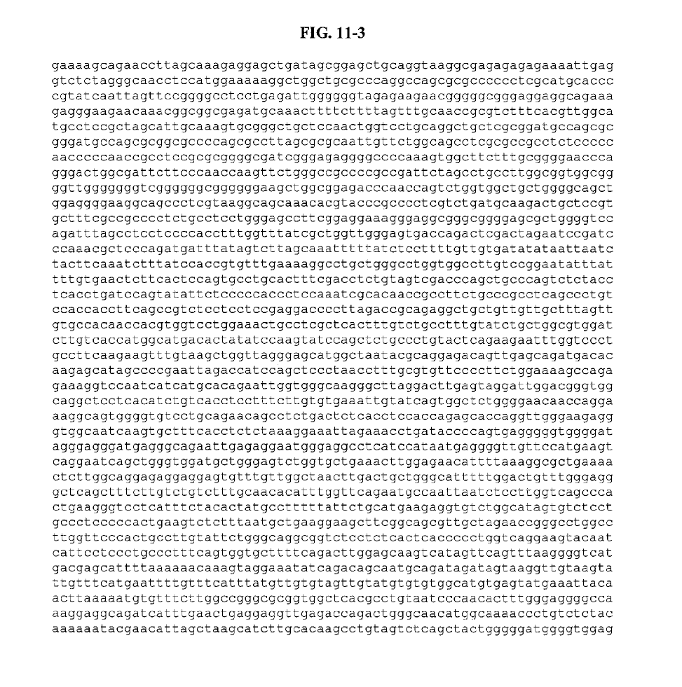

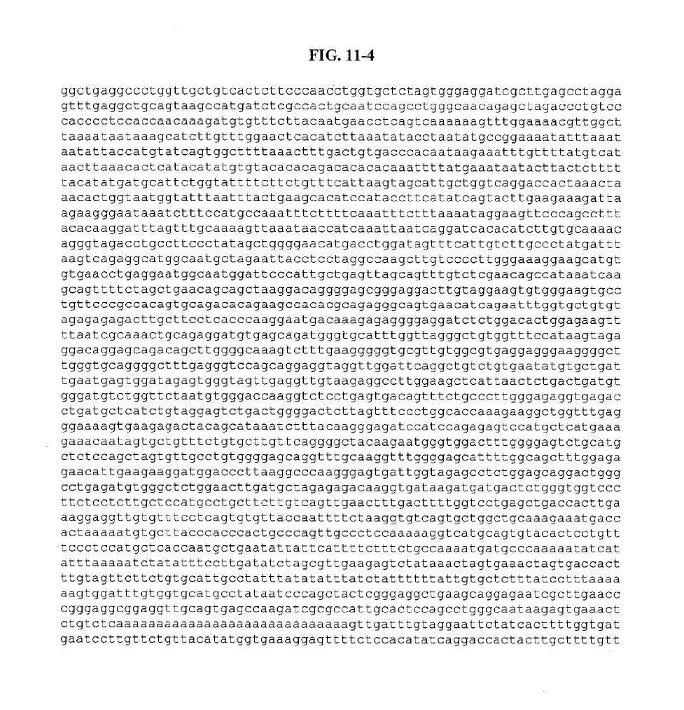

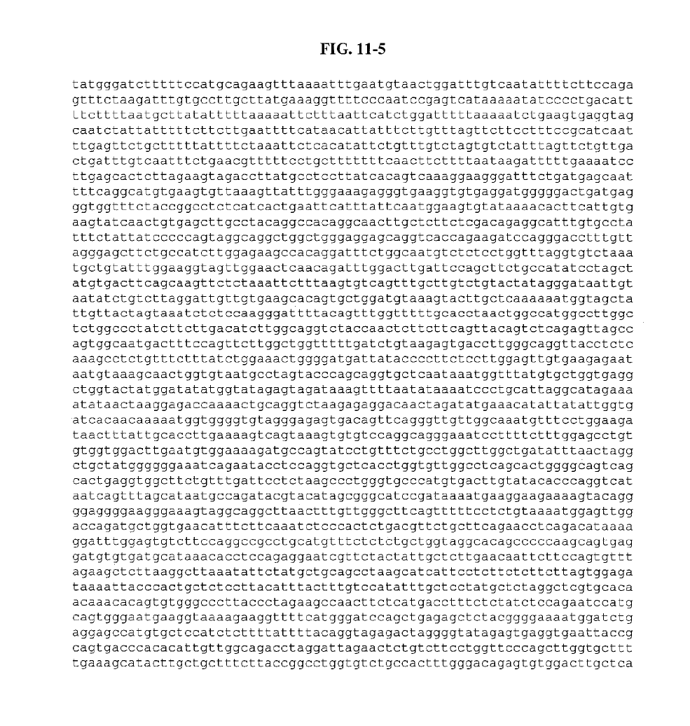

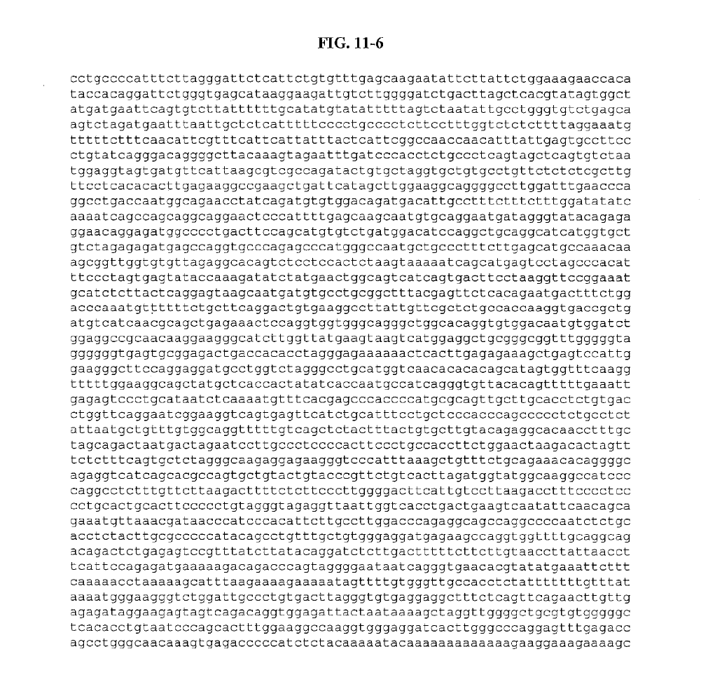

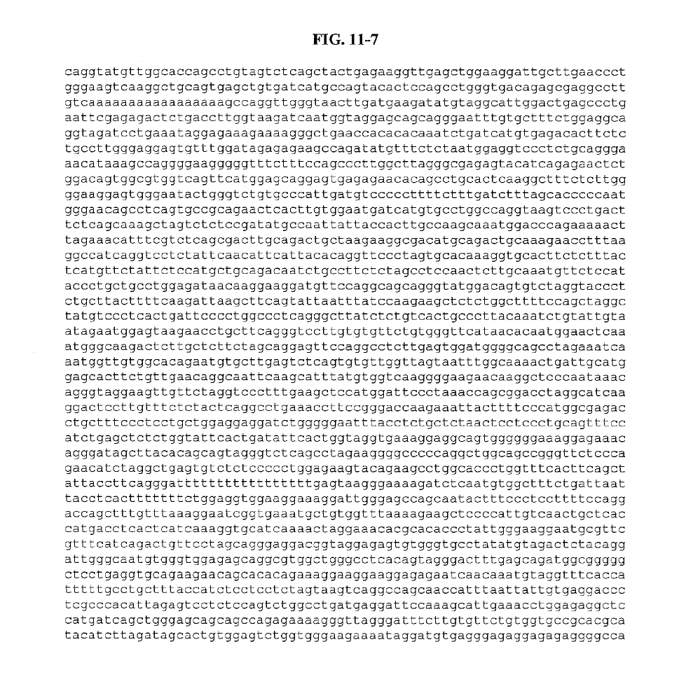

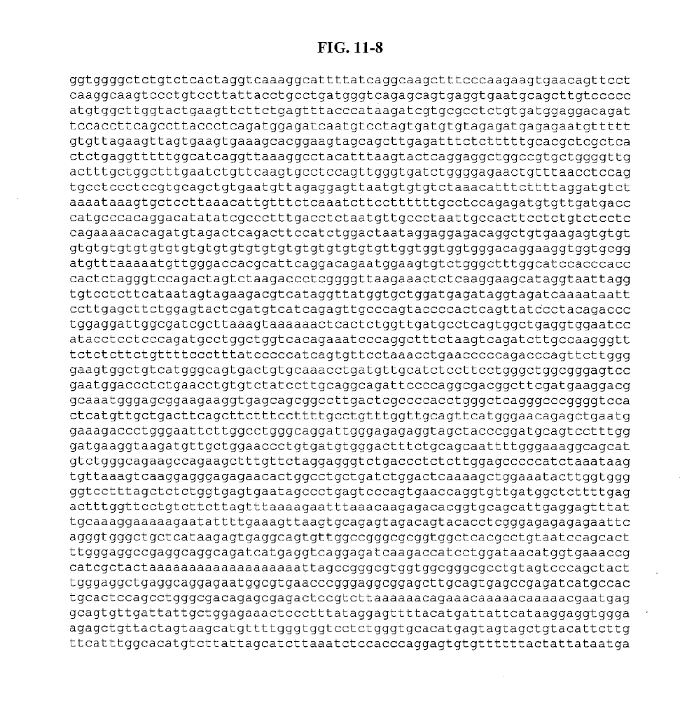

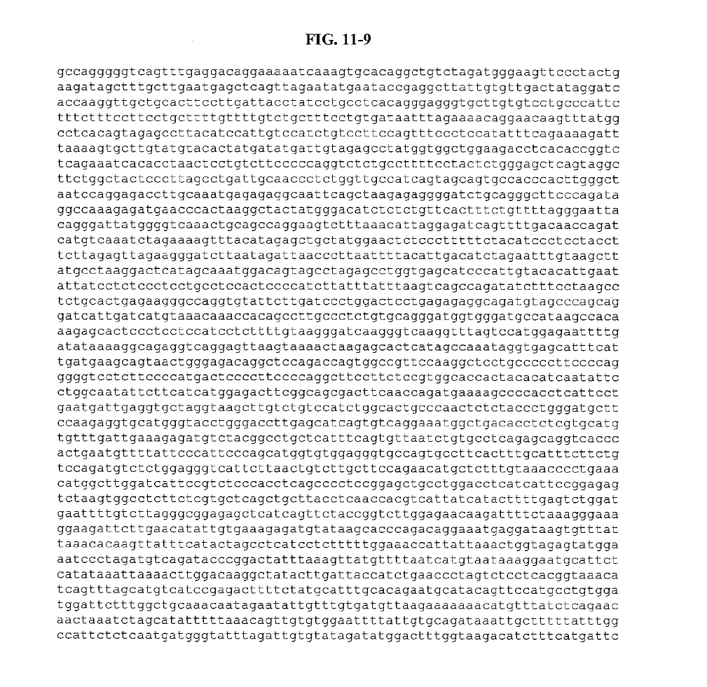

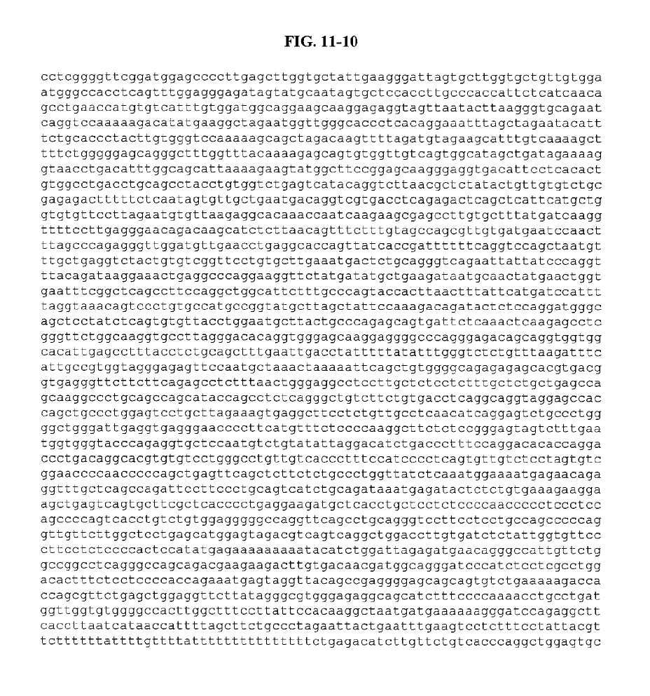

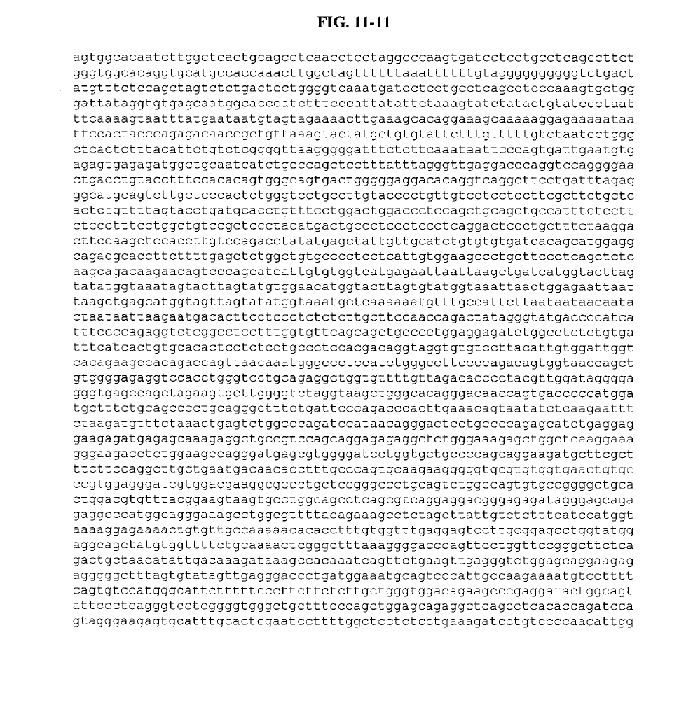

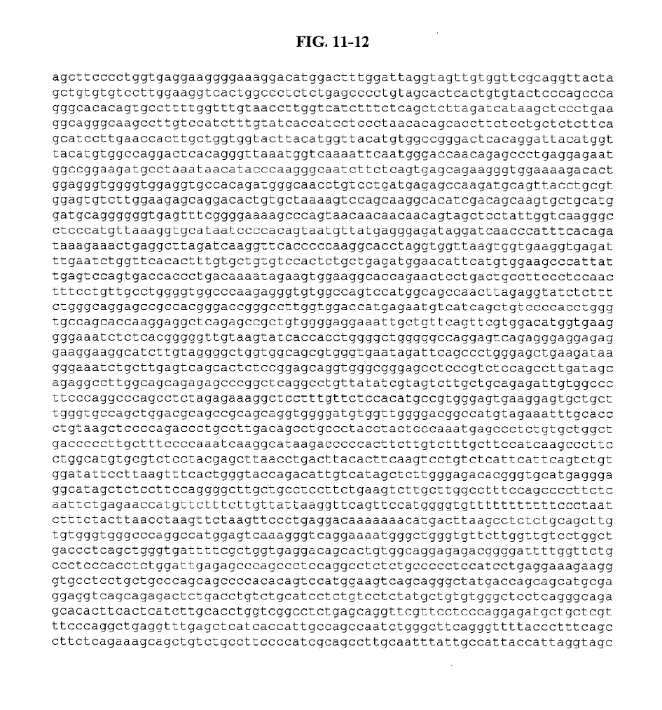

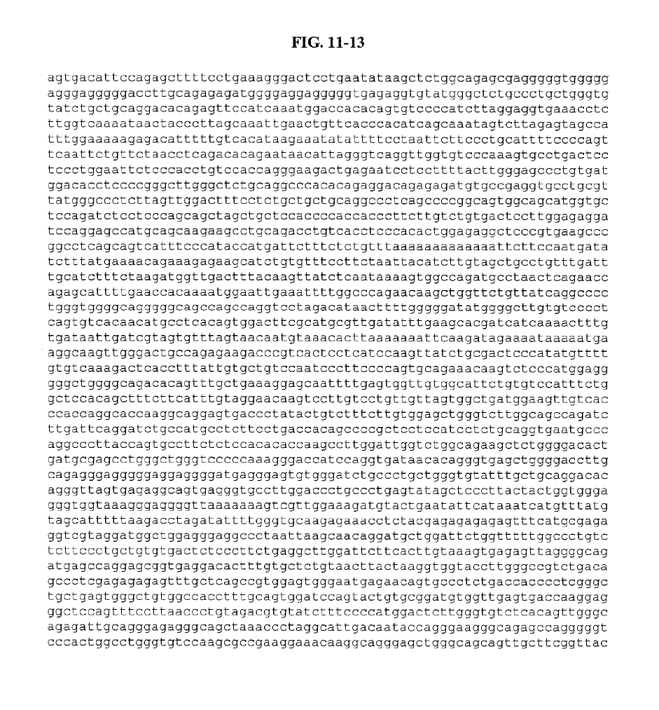

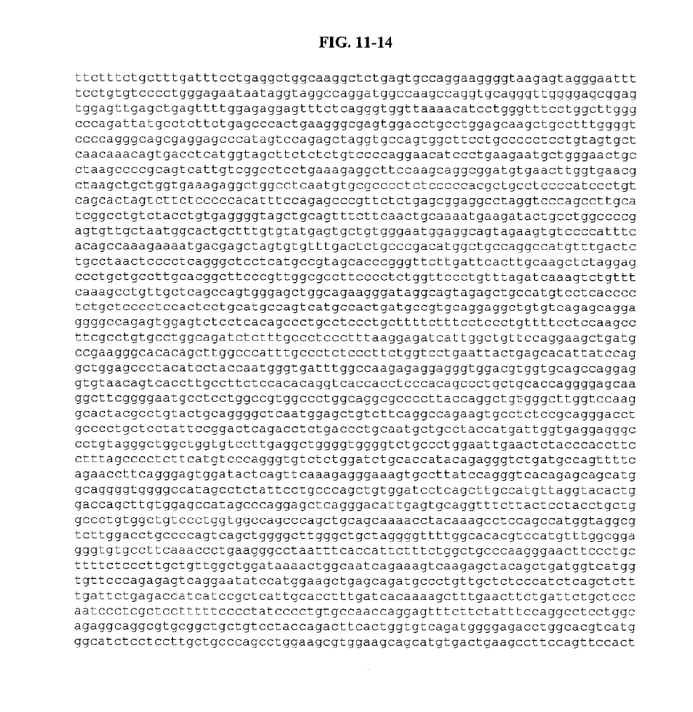

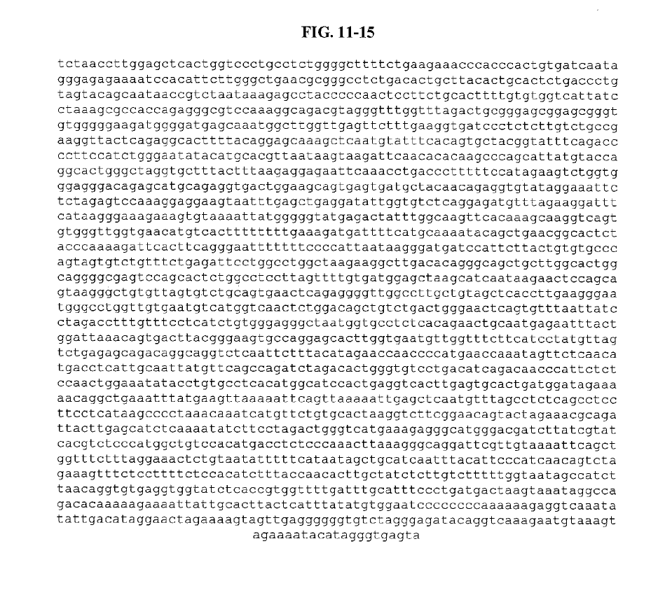

PHGDH encodes an enzyme that oxidizes 3-PG and has been reported to reduce NAD⁺ in vertebrates. Because cancer cells require large amounts of NADPH (Vander Heiden et al., Science 324: 1029-1033, 2009), PHGDH and the serine synthesis pathway may be providing NADPH for proliferating cells. Accordingly, we expressed PHGDH in bacteria and tested the ability of PHGDH to use NAD⁺ as a cofactor. His-tagged human PHGDH was subcloned into an IPTG-inducible pET vector for bacterial expression and transformed into an E. coli BL21 strain. Two liters of bacterial culture was grown to an 0D₆₀₀ of ˜0.7, and IPTG was added to induce expression of recombinant PHGDH. Recombinant PHGDH was purified from E. coli using a single-step His-tag purification with imidazole elution. PHGDH was dialyzed overnight, and aliquots of protein were snap frozen and stored at ˜80° C. We found that at high concentrations of 3-PG, PHGDH reduced NAD⁺ to form NADH (FIG. 10A). We then tested whether PHGDH could reduce NADP⁺. The ability to form NADH or NADPH was monitored by following the fluorescence of the reduced nicotinamide of NADH or NADPH at 340 nm. Recombinant PHGDH could convert either NAD⁺ or NADP⁺ to NADH or NADPH, respectively, as measured by reduced nicotinamide fluorescence. We demonstrated that PHGDH can convert NADP to NADPH at physiological concentrations of NADP⁺ (FIG. 10B).

We then showed that, using radio-isotopic tracers, glucose flux, specifically through the serine synthesis pathway, generates NADPH in cells. 5-³H-Glucose tracing was purchased from Perkin-Elmer. Exponentially-growing HEK293T cells were incubated with 5-³H-glucose. Cells were extracted using a 80:20 methanol:water mixture and metabolites separated by ion-pair chromatography. The reproducible separation of NADH and NADPH was determined using known standards and absorbance at 340 nm (FIG. 10C). Chromatography fractions from 5-³H-Glucose-labeled cell extracts were collected and radioactivity detected by scintillation counting. For confirmation of the NADPH peak, a co-injection of the cell extract with a ³H-labeled NADPH standard was performed (FIG. 10D). No radioactivity was found in the fractions corresponding to NADH elution. These data show that PHGDH is a critical generator of NADPH in proliferating cells and that inhibition of PHGDH has a detrimental effect on cell proliferation.

FIG. 10E shows the crystal structure of human PHGDH bound to NAD⁺ and its NADP⁺-utilizing homolog glyoxylate reductase. There is homology between glyoxylate reductase and PHGDH in the loop where the phosphate group distinguishing NADP from NAD would be located when NADP was bound to PHGDH, providing a structural rationale that NADP use as a cofactor is feasible.

### Example 6

### Tumor Microarray Data Sets in Breast Cancer

A study in breast cancer found enhanced high PHGDH mRNA expression was associated with poor prognosis in breast cancer (Pollari et al., Breast Cancer Res Treat. (2010)). Copy number gain was also found in breast cancer but at low frequency and in a broad peak region. To further investigate the role of PHGDH in breast cancer, we first carried out a bioinformatics analysis of multiple tumor microarray data sets in breast cancer and found strong associations (p<1 e-4) with several clinical parameters in breast cancer. These data suggest that PHGDH expression segregated with specific cancer subtypes. For validation, PHGDH protein expression in 106 human breast cancer tumor samples was assessed by IHC and correlated with mRNA expression. It was found that high PHGDH expression (IHC score >1) was associated with distinct subtypes of breast cancer, as expression correlated with both triple-negative (Foulkes et al., New England Journal of Medicine 363(2010)) (p=0.002, Fisher's exact, two tailed) and basal subtypes (p=0.004, Fisher's exact, two tailed). However, there was no association with general parameters such as metastasis as was previously reported (Pollari et al., Breast Cancer Res Treat. (2010)) or with tumor size, suggesting that expression is subtype specific in breast cancer.

Consistent with a reliance of a subset of breast cancers on PHGDH, protein expression was required for growth in a panel of three (BT-20, SK-BR-3, MCF-7) breast cancer cell lines (including the BT-20 cell line that carries amplification) to differing extents. Furthermore, decreased PHGDH expression decreased pSer levels in PHGDH amplified BT-20 cells. In contrast, non-tumorigenic breast epithelial cells (MCF-10a) did not require PHGDH for growth, did not exhibit alterations in glycolysis upon shRNA knockdown of PHGDH and exhibited no detectable labeling of pSER from glucose.

### Example 7

### Ectopic Expression of PHGDH would Increase Flux of Glucose to Serine and have any Phenotypic Consequences

We questioned whether ectopic expression of PHGDH would increase flux of glucose to serine and have any phenotypic consequences. MCF-10a cells are non-tumorigenic and, when grown in reconstituted basement membrane (™Matrigel) form structures resembling many features of mammary acini. These acini-like structures are polarized and characterized by a hollow lumen due to selective apoptosis of the inner, matrix-deprived cells. This model has been used to monitor alterations in growth arrest, polarization, invasive behavior and other disruptions of normal morphogenesis that resemble changes associated with different stages of tumor formation (Debnath et al., Nature Reviews Cancer 5, 675-688 (2005)).

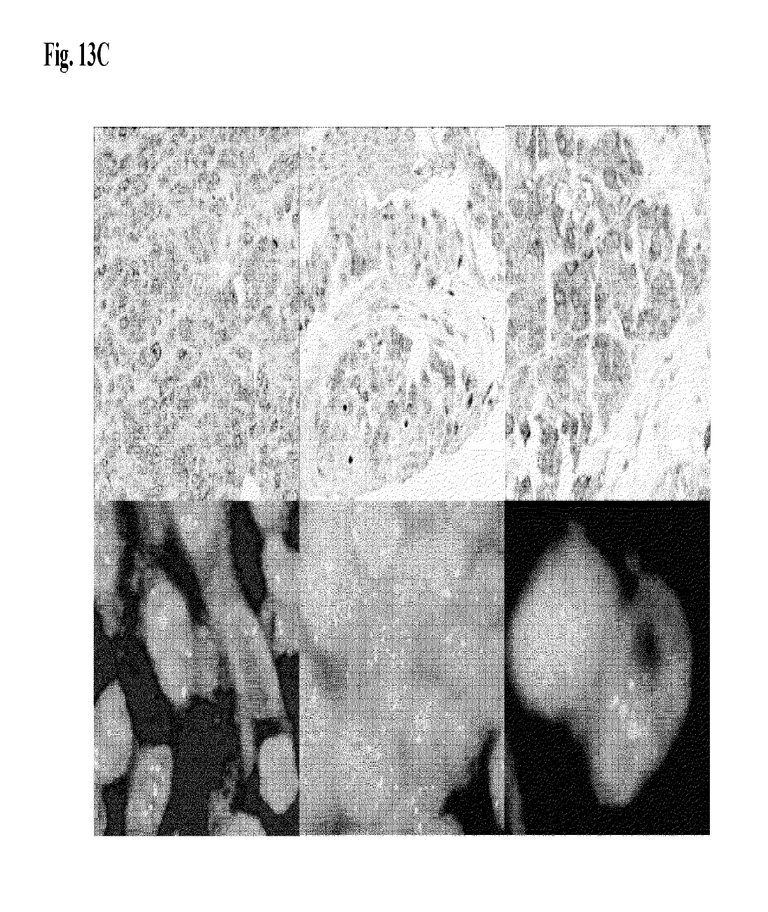

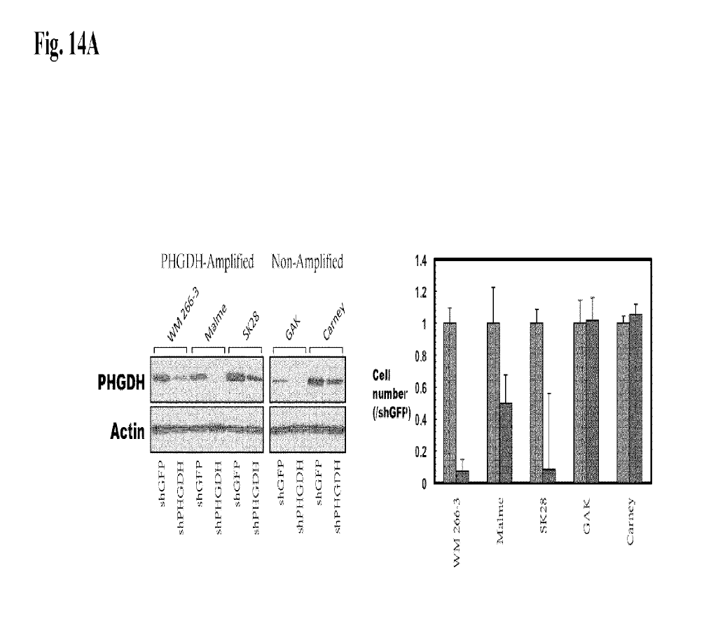

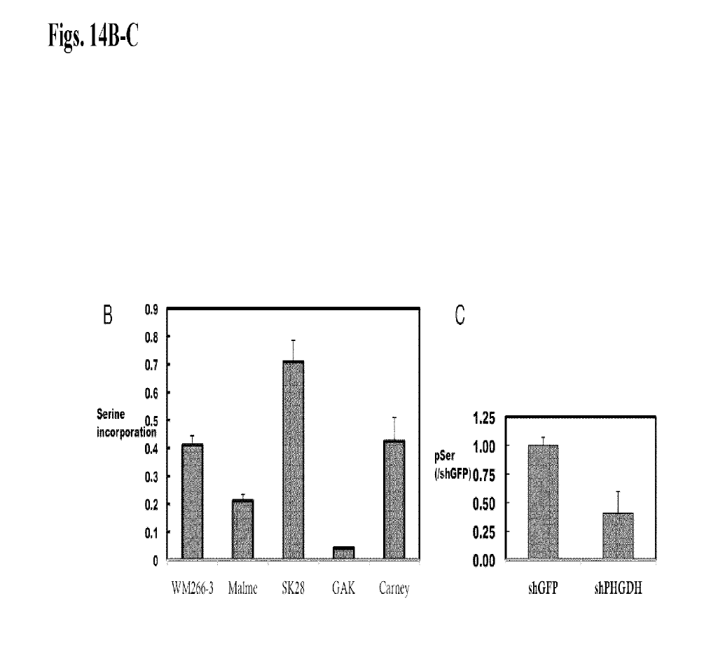

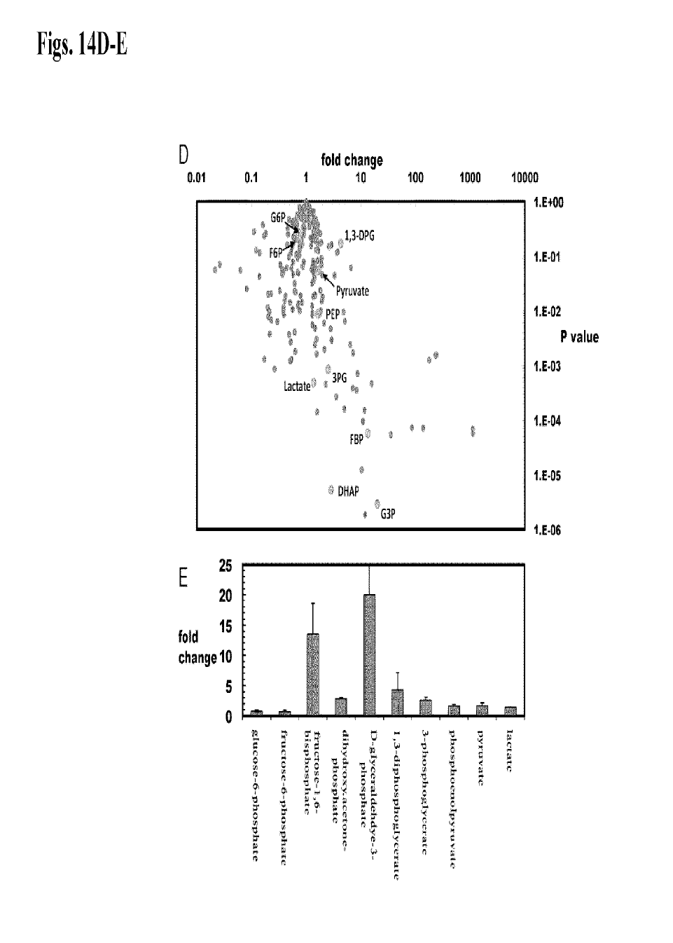

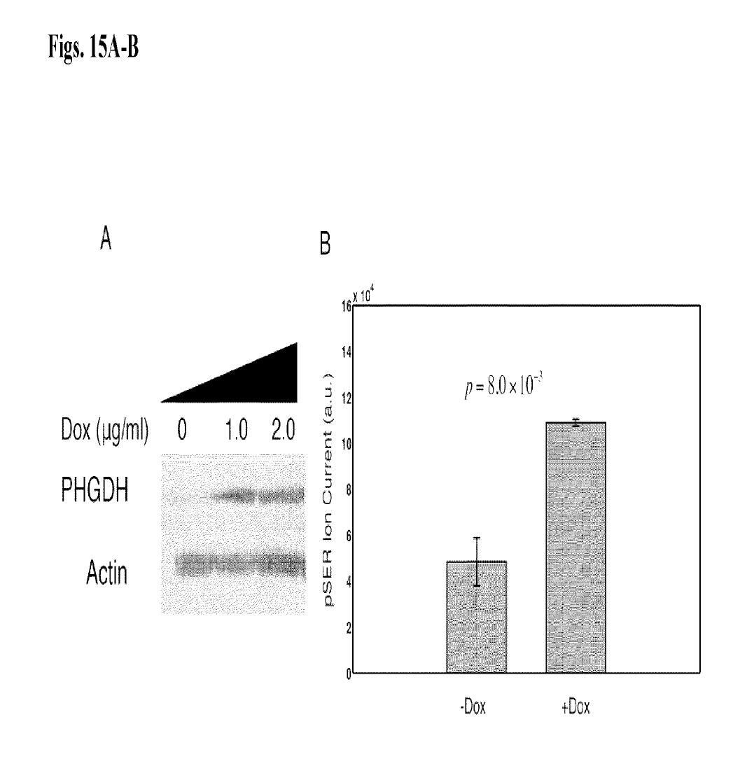

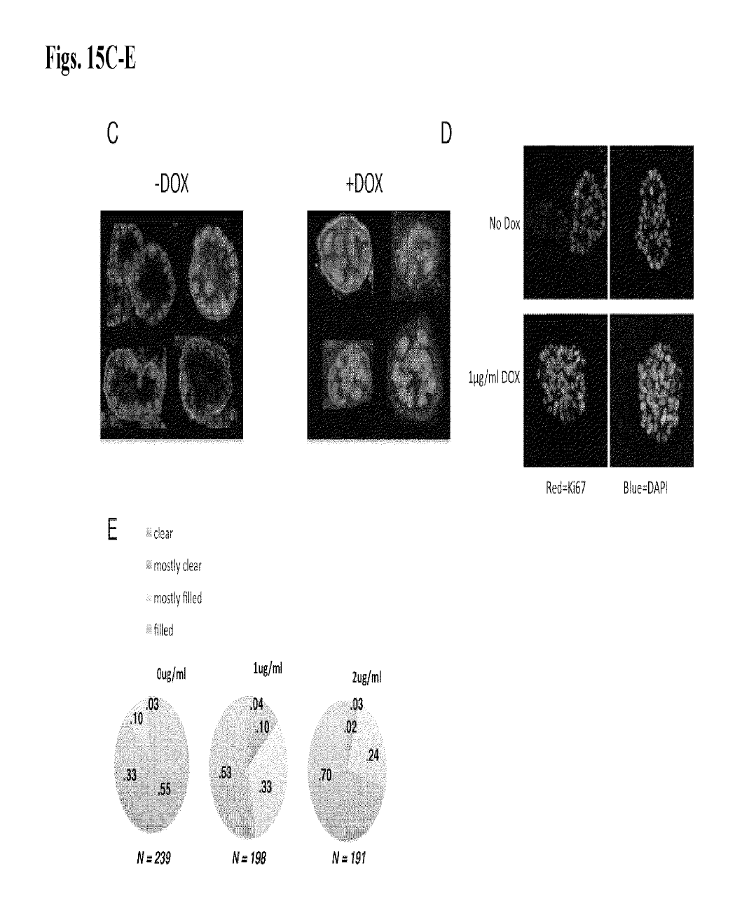

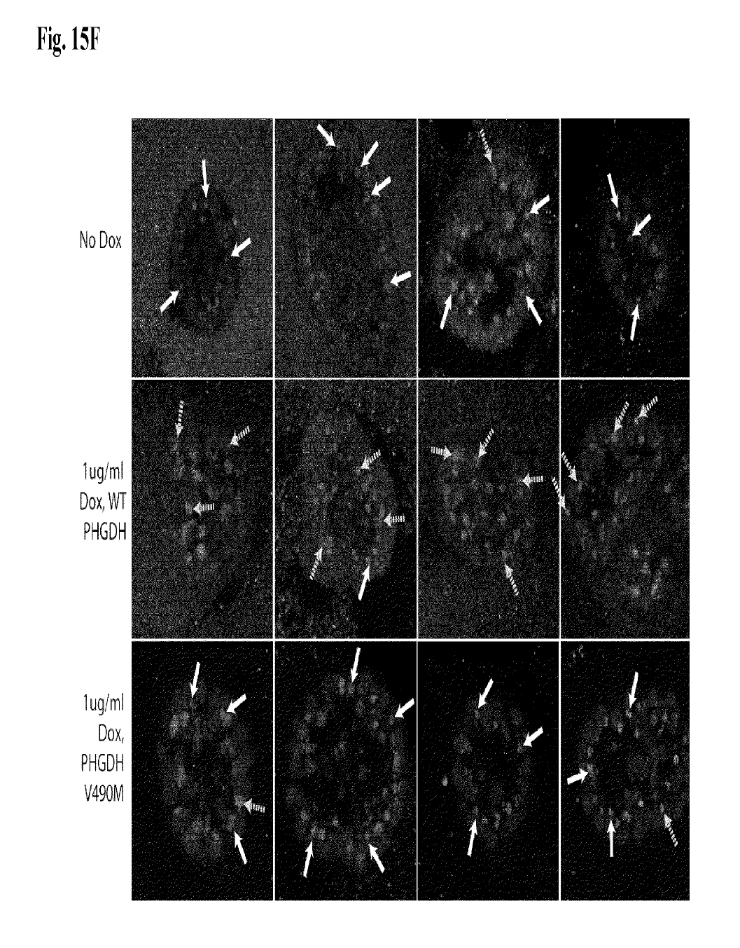

PHGDH was expressed in MCF-10a cells using a tetracycline-inducible expression vector and treatment of the engineered MCF-10A cells with increasing concentrations of doxycycline induced expression of PHGDH (FIG. 15A). pSER levels were elevated to detectable levels in cells treated with 1 μg/ml doxycycline indicating an increase in pathway activity (FIG. 15B) that was confirmed with GC/MS that measured an increase in serine and glycine synthesis.

We seeded PHGDH-expressing MCF-10A cells in ™Matrigel reconstituted basement membrane and monitored the structures at increasing doses of doxycycline using confocal microscopy and immunofluorescence staining of nuclei (DAPI) and extracellular matrix (laminin-5) (FIG. 15C). In the absence of doxycycline, MCF-10A cells formed hollow, acini-like structures as previously reported (Schafer et al., Nature 461, 109-U118 (2009)) (FIG. 15C). In contrast, PHGDH-expressing cells formed disorganized structures lacking a lumen (FIG. 15C). The PHGDH-expressing cells also exhibited large, abnormal nuclear morphologies, failed to orient in a uniform fashion adjacent to the basal acinar membrane, and displayed enhanced proliferation (FIG. 15D). The majority of the control acini were either clear or mostly clear, whereas PHGDH expression dramatically increased the percentage of acini that scored as mostly filled or filled in a dose dependent manner (FIG. 4E). An activity-compromised mutant PHGDH (V490M) (Tabatabaie et al., Human Mutation 30, 749-756 (2009)) showed decreased luminal filling (FIG. 15F). In addition, MCF-10A acini with ectopic expression of wild-type but not mutant PHGDH commonly displayed mislocalization of the golgi apparatus indicating loss of apical polarity (FIG. 15F). These results indicate that PHGDH expression alters glucose metabolism, disrupts luminal organization and polarity and preserves the viability of the inner, matrix-deprived cells to survive in an anchorage-independent fashion. These phenotypes depend on the catalytic activity of PHGDH.

### Example 8

### Screening Methods for Identifying Inhibitors of Enzymes of the Serine Biosynthetic Pathway

We have discovered that inhibition of PHGDH inhibits the production of NADPH and cell proliferation. Accordingly, the present invention features methods and compositions for the treatment of cellular proliferative disorders (e.g., cancer and obesity) by targeting enzymes of the serine biosynthetic pathway (e.g., PHGDH, phosphoserine aminotransferase (PSAT), or phosphoserine phosphatase (PSPH)).

To identify inhibitors of PHGDH, PHGDH enzyme activity (e.g., full-length PHGDH or a functional fragment thereof) is coupled in a screen with a 10-fold excess of PSAT (e.g., full-length PSAT or a functional fragment thereof) and/or PSPH (e.g., full-length PSPH or a functional fragment thereof), 100 μM of glutamate, glucose, 3-phosphoglycerate (3-PG), and NADP⁺. This coupled system is then used to screen for inhibitors of PHGDH by monitoring the conversion of NADP⁺ to NADPH in the presence of 3-PG. The conversion of NADP to NADPH may be monitored through fluorescence spectroscopy.

In another example, NADPH production is measured by coupling the reaction of 3-PG with PHGDH and PSAT (i.e., 3-hydroxypyruvate, 3-phosphoserine, and serine) to enzymes whose activities allow for high-throughput monitoring, for example, through fluorescence or hydrogen peroxide.

In another example, cells expressing PHGDH can be treated with a 10-fold excess of PSAT and/or PSPH, 100 μM of glutamate, glucose, 3-phosphoglycerate (3-PG), and NADP⁺. The cells are then treated with a candidate compound (e.g., a peptide, nucleic acid molecule, aptamer, small molecule, or polysaccharide). Control cells are not treated with the candidate compound. Candidate compounds that inhibit PHGDH inhibit the conversion of NADP⁺ to NADPH. Candidate compounds that do not inhibit PHGDH do not inhibit the conversion of NADP⁺ to NADPH. A decrease in the level of NADPH in a cell contacted with the candidate compound compared to a cell not contacted with the candidate compound identifies the candidate compound as an inhibitor of PHGDH.

Decreases in nucleotide metabolism are also monitored in cell-based assays, as PHGDH coordinates nucleotide metabolism in downstream pathways. Such decreases are monitored with fluorescence-based assays.

Additional screening assays are performed to monitor the expression of PHGDH or the biological activity of PHGDH (e.g., the catalysis of 3-phosphoglycerate to 3-phosphohydroxypyruvate or the promotion of cell proliferation). A reduction in the expression of PHGDH or a reduction in the biological activity of PHGDH upon administration of a candidate compound indicates that the compound may be an inhibitor of PHGDH.

### Other Embodiments

From the foregoing description, it will be apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent application, or patent was specifically and individually indicated to be incorporated by reference.

From the foregoing description, one skilled in the art can easily ascertain the essential characteristics of this invention; can make various changes and modifications of the invention to adapt it to various usages and conditions. Thus, other embodiments are also within the claims.

What is claimed is:

1.-8. (canceled)

9. A method for diagnosing a cellular proliferative disorder in a subject or assigning a prognostic risk of developing a cellular proliferative disorder in a subject, said method comprising determining a phosphoglycerate dehydrogenase (PHGDH) gene copy number in a biological sample from said subject, wherein an amplification of the PHGDH gene in said biological sample from said subject relative to a control gene copy number indicates the presence of a cellular proliferative disorder in said subject or the risk of developing said cellular proliferative disorder in said subject.

10. The method of claim 9, wherein said PHGDH copy number is increased by at least 3-fold.

11. The method of claim 9, wherein said PHGDH gene copy number is determined by a hybridization-assay and/or an amplification-based assay.

12. The method of claim 9, wherein said PHGDH gene copy number is determined by fluorescence in situ hybridization (FISH).

13. The method of claim 9, wherein said PHGDH gene copy number is determined by comparative genomic hybridization (CGH).

14. The method of claim 9, wherein said PHGDH gene copy number is determined by microarray-based CGH.

15. A method of identifying an inhibitor of phosphoglycerate dehydrogenase (PHGDH), said method comprising:
(a) contacting a cell that expresses PHGDH with a candidate compound; and
(b) determining a level of NADPH present in said cell contacted with said candidate compound, wherein a reduction in the level of NADPH in said cell contacted with said candidate compound compared to a level of NADPH in a control cell not contacted with said candidate compound identifies said candidate compound as an inhibitor of PHGDH.

16. The method of claim 15, wherein said cell has an excess of phosphoserine aminotransferase.

17. The method of claim 15, wherein said cell has an excess of glutamate.

18. A method of identifying an inhibitor of phosphoglycerate dehydrogenase (PHGDH), said method comprising:
(a) contacting a sample comprising PHGDH, or a functional fragment thereof, and NADP⁺ with a candidate compound; and
(b) determining a level of NADPH present in said sample, wherein a reduction in the level of NADPH in said sample contacted with said candidate compound compared to a level of NADPH in a control sample not contacted with said candidate compound identifies said candidate compound as an inhibitor of PHGDH.

19. The method of claim 18, wherein said sample contacted with said candidate compound further comprises phosphoserine aminotransferase and/or glutamate.

20.-32. (canceled)

33. The method of claim 15, wherein said determining step is performed using fluorescence spectroscopy.

34. The method of claim 18, wherein said determining step is performed using fluorescence spectroscopy.
